(19) United States
(12) Patent Application Publication
(10) Pub. No.: US 2026/0255784 A1
(43) Pub. Date: Aug. 27, 2026

(54) MATERIALS FOR ELECTRONIC DEVICES

(71) Applicant: Merck Patent GmbH, Darmstadt (DE)

(72) Inventors: Teresa MUJICA-FERNAUD, Darmstadt (DE); Elvira MONTENEGRO, Darmstadt (DE); Christian WIRGES, Darmstadt (DE)

(21) Appl. No.: 18/994,146

(22) PCT Filed: Jul. 13, 2023
(86) PCT No.: PCT/EP2023/069421

§ 371 Date: Jan. 14, 2025

(30) Foreign Application Priority Data
Jul. 15, 2022 (EP) 22185094.4
Aug. 12, 2022 (EP) 22190277.8

(51) Int. Cl. H10K 50/15; C07B 59/00; C07C 211/54; H10K 50/11; H10K 85/30; H10K 85/60

(52) U.S. Cl. CPC H10K 50/15; C07B 59/002; C07C 211/54; H10K 50/11; H10K 85/342; H10K 85/346; H10K 85/622; H10K 85/626; H10K 85/633; H10K 85/636; H10K 85/654; H10K 85/6572; H10K 85/6574; C07B 2200/05

(57) ABSTRACT

The present invention relates to a compound of formula (I), to the use of the compound in an electronic device, to a method for producing said compound, and to an electronic device containing the compound.

The present application relates to aromatic amines having particular aromatic or heteroaromatic ring systems on the amine nitrogen atom. The compounds are suitable for use in electronic devices.

Electronic devices in the context of this application are understood to mean what are called organic electronic devices, which comprise organic semiconductor materials as functional materials. More particularly, these are understood to mean OLEDs (organic electroluminescent devices). The term OLEDs is understood to mean electronic devices which have one or more layers comprising organic compounds and emit light on application of electrical voltage. The structure and general principle of function of OLEDs are known to those skilled in the art.

In electronic devices, especially OLEDs, there is great interest in an improvement in the performance data. In these aspects, it has not yet been possible to find any entirely satisfactory solution.

A major influence on the performance data of electronic devices is possessed by emission layers and layers having a hole-transporting function. There is an ongoing search for novel compounds for use in these layers, especially hole-transporting compounds and compounds that can serve as hole-transporting matrix material, especially for phosphorescent emitters, in an emitting layer. For this purpose, there is a search in particular for compounds that have a high glass transition temperature, high stability, and high conductivity for holes. A high stability of the compound is a prerequisite for achieving a long lifetime of the electronic device. There is also a search for compounds whose use in electronic devices results in improvement of the performance data of the devices, especially in high efficiency, long lifetime and low operating voltage.

In the prior art, triarylamine compounds in particular, for example spirobifluoreneamines and fluoreneamines, are known as hole transport materials and hole-transporting matrix materials for electronic devices.

However, there remains room for improvement in respect of the abovementioned properties.

It has now been found that aromatic amines of the formula below which are characterized in that they have particular aromatic or heteroaromatic ring systems on the amine nitrogen atom are of excellent suitability for use in electronic devices. They are especially suitable for use in OLEDs, and even more particularly therein for use as hole transport materials and for use as hole-transporting matrix materials, especially for phosphorescent emitters. The compounds lead to high lifetime, high efficiency and low operating voltage of the devices. Further preferably, the compounds found have a high glass transition temperature, high stability, low sublimation temperature, good solubility, good synthetic accessibility and high conductivity for holes.

The present application thus provides a compound of a formula (I)

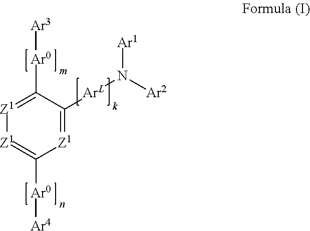

where the variables that occur are as follows:
Z¹ is the same or different at each instance and is selected from N and CR¹; where the unit

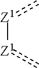

may be replaced by a unit selected from units of the following formulae:

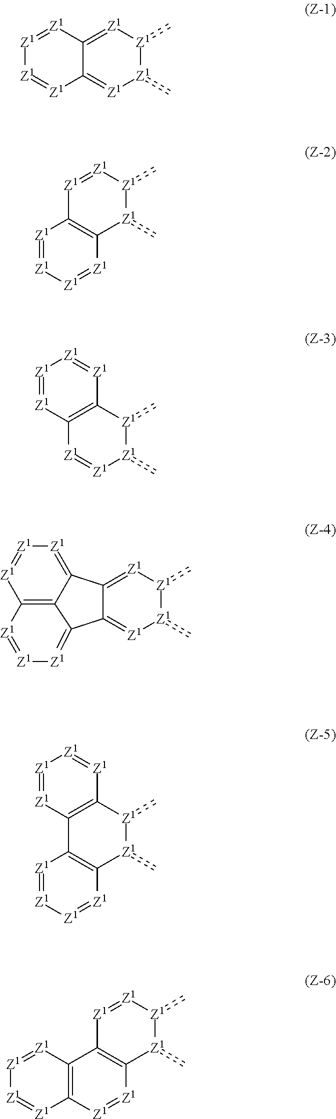

where the dotted bonds in the units each correspond to the bonds in the unit

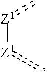

and where the variables Z¹ in the unit

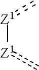

in these cases are C;
Ar⁰ is phenylene substituted by R² radicals;
Ar¹ and Ar²
   are the same or different at each instance and are selected from aromatic ring systems which have 6 to 40 aromatic ring atoms and are substituted by R⁴ radicals, and from heteroaromatic ring systems which have 5 to 40 aromatic ring atoms and are substituted by R⁴ radicals;
Ar³ and Ar⁴
   are the same or different at each instance and are selected from aromatic ring systems which have 6 to 40 aromatic ring atoms and are substituted by R⁵ radicals, and from heteroaromatic ring systems which have 5 to 40 aromatic ring atoms and are substituted by R⁵ radicals;
   where at least one of Ar³ and Ar⁴ is selected from aryl groups which have 10 to 20 aromatic ring atoms and are substituted by R⁵ radicals;
Ar^{L} is selected from aromatic ring systems which have 6 to 40 aromatic ring atoms and are substituted by R³ radicals and from heteroaromatic ring systems which have 5 to 40 aromatic ring atoms and are substituted by R³ radicals;
R¹ is the same or different at each instance and is selected from H, D, F, Cl, Br, I, C(═O)R⁶, CN, Si(R⁶)₃, N(R⁶)₂, P(═O)(R⁶)₂, OR⁶, S(═O)R⁶, S(═O)₂R⁶, straight-chain alkyl or alkoxy groups having 1 to 20 carbon atoms, branched or cyclic alkyl or alkoxy groups having 3 to 20 carbon atoms, alkenyl or alkynyl groups having 2 to 20 carbon atoms, where said alkyl, alkoxy, alkenyl and alkynyl groups are each substituted by R⁶ radicals; and where one or more CH₂ groups in said alkyl, alkoxy, alkenyl and alkynyl groups may be replaced by —R⁶C≡CR⁶—, —C≡C—, Si(R⁶)₂, C═O, C≡NR⁶, —C(═O)O—, —C(═O)NR⁶—, NR⁶, P(═O)(R⁶), —O—, —S—, SO or SO₂;
R⁴ is the same or different at each instance and is selected from H, D, F, Cl, Br, I, C(═O)R⁶, CN, Si(R⁶)₃, P(═O)(R⁶)₂, OR⁶, S(═O)R⁶, S(═O)₂R⁶, straight-chain alkyl or alkoxy groups having 1 to 20 carbon atoms, branched or cyclic alkyl or alkoxy groups having 3 to 20 carbon atoms, alkenyl or alkynyl groups having 2 to 20 carbon atoms, aromatic ring systems having 6 to 40 aromatic ring atoms, and heteroaromatic ring systems having 5 to 40 aromatic ring atoms; where two or more R⁴ radicals may be joined to one another and may form a ring; where said alkyl, alkoxy, alkenyl and alkynyl groups and said aromatic ring systems and heteroaromatic ring systems are each substituted by R⁶ radicals; and where one or more CH₂ groups in said alkyl, alkoxy, alkenyl and alkynyl groups may be replaced by —R⁶C≡CR⁶—, —C≡C—, Si(R⁶)₂, C═O, C≡NR⁶, —C(═O)O—, —C(═O)NR⁶—, NR⁶, P(═O)(R⁶), —O—, —S—, SO or SO₂;
R², R³ and R⁵
   are the same or different at each instance and are selected from H, D, F, Cl, Br, I, C(═O)R⁶, CN, Si(R⁶)₃, N(R⁶)₂, P(═O)(R⁶)₂, OR⁶, S(═O)R⁶, S(═O)₂R⁶, straight-chain alkyl or alkoxy groups having 1 to 20 carbon atoms, branched or cyclic alkyl or alkoxy groups having 3 to 20 carbon atoms, alkenyl or alkynyl groups having 2 to 20 carbon atoms, aromatic ring systems having 6 to 40 aromatic ring atoms, and heteroaromatic ring systems having 5 to 40 aromatic ring atoms; where two or more radicals selected from the R², R³ and R⁵ radicals may be joined to one another and may form a ring; where said alkyl, alkoxy, alkenyl and alkynyl groups and said aromatic ring systems and heteroaromatic ring systems are each substituted by R⁶ radicals; and where one or more CH₂ groups in said alkyl, alkoxy, alkenyl and alkynyl groups may be replaced by —R⁶C≡CR⁶—, —C≡C—, Si(R⁶)₂, C═O, C≡NR⁶, —C(═O)O—, —C(═O)NR⁶—, NR⁶, P(═O)(R⁶), —O—, —S—, SO or SO₂;
R⁶ is the same or different at each instance and is selected from H, D, F, Cl, Br, I, C(═O)R⁷, CN, Si(R⁷)₇, P(═O)(R⁷)₂, OR⁷, S(═O)R⁷, S(═O)₂R⁷, straight-chain alkyl or alkoxy groups having 1 to 20 carbon atoms, branched or cyclic alkyl or alkoxy groups having 3 to 20 carbon atoms, alkenyl or alkynyl groups having 2 to 20 carbon atoms, aromatic ring systems having 6 to 40 aromatic ring atoms, and heteroaromatic ring systems having 5 to 40 aromatic ring atoms; where two or more R⁶ radicals may be joined to one another and may form a ring; where the alkyl, alkoxy, alkenyl and alkynyl groups mentioned and the aromatic ring systems and heteroaromatic ring systems mentioned are each substituted by R³ radicals; and where one or more CH₂ groups in the alkyl, alkoxy, alkenyl and alkynyl groups mentioned may be replaced by —R⁷C≡CR⁷—, —C≡C—, Si(R⁷)₂, C═O, C≡NR⁷, —C(═O)O—, —C(═O)NR⁷—, NR⁷, P(═O)(R⁷), —O—, —S—, SO or SO₂;
R⁷ is the same or different at each instance and is selected from H, D, F, Cl, Br, I, CN, alkyl or alkoxy groups having 1 to 20 carbon atoms, alkenyl or alkynyl groups having 2 to 20 carbon atoms, aromatic ring systems having 6 to 40 aromatic ring atoms and heteroaromatic ring systems having 5 to 40 aromatic ring atoms; where two or more R⁷ radicals may be joined to one another and may form a ring; and where said alkyl, alkoxy, alkenyl and alkynyl groups, aromatic ring systems and heteroaromatic ring systems may be substituted by one or more radicals selected from F and CN
m is 0, 1, 2 or 3;
n is 0, 1, 2 or 3;
k is 0 or 1.

When n or m is 0, the groups bonded to the Ar⁰ group in question are bonded directly to one another, and the Ar⁰ group in question is absent.

When k is 0, the groups bonded to the Ar^{L} group are bonded directly to one another, and the Ar^{L} group is absent.

The definitions which follow are applicable to the chemical groups that are used in the present application. They are applicable unless any more specific definitions are given.

An aryl group in the context of this invention is understood to mean either a single aromatic cycle, i.e. benzene, or a fused aromatic polycycle, for example naphthalene, phenanthrene or anthracene. A fused aromatic polycycle in the context of the present application consists of two or more single aromatic cycles fused to one another. Fusion between cycles is understood here to mean that the cycles share at least one edge with one another. An aryl group in the context of this invention contains 6 to 40 aromatic ring atoms. In addition, an aryl group does not contain any heteroatom as aromatic ring atom, but only carbon atoms.

A heteroaryl group in the context of this invention is understood to mean either a single heteroaromatic cycle, for example pyridine, pyrimidine or thiophene, or a fused heteroaromatic polycycle, for example quinoline or carbazole. A fused heteroaromatic polycycle in the context of the present application consists of two or more single aromatic or heteroaromatic cycles that are fused to one another, where at least one of the aromatic and heteroaromatic cycles is a heteroaromatic cycle. Fusion between cycles is understood here to mean that the cycles share at least one edge with one another. A heteroaryl group in the context of this invention contains 5 to 40 aromatic ring atoms of which at least one is a heteroatom. The heteroatoms of the heteroaryl group are preferably selected from N, O and S.

An aryl or heteroaryl group, each of which may be substituted by the abovementioned radicals, is especially understood to mean groups derived from benzene, naphthalene, anthracene, phenanthrene, pyrene, dihydropyrene, chrysene, perylene, triphenylene, fluoranthene, benzanthracene, benzophenanthrene, tetracene, pentacene, benzopyrene, furan, benzofuran, isobenzofuran, dibenzofuran, thiophene, benzothiophene, isobenzothiophene, dibenzothiophene, pyrrole, indole, isoindole, carbazole, pyridine, quinoline, isoquinoline, acridine, phenanthridine, benzo-5,6-quinoline, benzo-6,7-quinoline, benzo-7,8-quinoline, phenothiazine, phenoxazine, pyrazole, indazole, imidazole, benzimidazole, benzimidazolo [1,2-a]benzimidazole, naphthimidazole, phenanthrimidazole, pyridimidazole, pyrazinimidazole, quinoxalinimidazole, oxazole, benzoxazole, naphthoxazole, anthroxazole, phenanthroxazole, isoxazole, 1,2-thiazole, 1,3-thiazole, benzothiazole, pyridazine, benzopyridazine, pyrimidine, benzopyrimidine, quinoxaline, pyrazine, phenazine, naphthyridine, azacarbazole, benzocarboline, phenanthroline, 1,2,3-triazole, 1,2,4-triazole, benzotriazole, 1,2,3-oxadiazole, 1,2,4-oxadiazole, 1,2,5-oxadiazole, 1,3,4-oxadiazole, 1,2,3-thiadiazole, 1,2,4-thiadiazole, 1,2,5-thiadiazole, 1,3,4-thiadiazole, 1,3,5-triazine, 1,2,4-triazine, 1,2,3-triazine, tetrazole, 1,2,4,5-tetrazine, 1,2,3,4-tetrazine, 1,2,3,5-tetrazine, purine, pteridine, indolizine and benzothiadiazole.

An aromatic ring system in the context of this invention is a system which does not necessarily contain solely aryl groups, but which may additionally contain one or more nonaromatic rings fused to at least one aryl group. These nonaromatic rings contain exclusively carbon atoms as ring atoms. Examples of groups covered by this definition are tetrahydronaphthalene, fluorene and spirobifluorene. In addition, the term “aromatic ring system” includes systems that consist of two or more aromatic ring systems joined to one another via single bonds, for example biphenyl, terphenyl, 7-phenyl-2-fluorenyl, quaterphenyl and 3,5-diphenyl-1-phenyl. An aromatic ring system in the context of this invention contains 6 to 40 carbon atoms and no heteroatoms in the ring system. The definition of “aromatic ring system” does not include heteroaryl groups.

A heteroaromatic ring system conforms to the abovementioned definition of an aromatic ring system, except that it must contain at least one heteroatom as ring atom. As is the case for the aromatic ring system, the heteroaromatic ring system need not contain exclusively aryl groups and heteroaryl groups, but may additionally contain one or more nonaromatic rings fused to at least one aryl or heteroaryl group. The nonaromatic rings may contain exclusively carbon atoms as ring atoms, or they may additionally contain one or more heteroatoms, where the heteroatoms are preferably selected from N, O and S. One example of such a heteroaromatic ring system is benzopyranyl. In addition, the term “heteroaromatic ring system” is understood to mean systems that consist of two or more aromatic or heteroaromatic ring systems that are bonded to one another via single bonds, for example 4,6-diphenyl-2-triazinyl. A heteroaromatic ring system in the context of this invention contains 5 to 40 ring atoms selected from carbon and heteroatoms, where at least one of the ring atoms is a heteroatom. The heteroatoms of the heteroaromatic ring system are preferably selected from N, O and S.

The terms “heteroaromatic ring system” and “aromatic ring system” as defined in the present application thus differ from one another in that an aromatic ring system cannot have a heteroatom as ring atom, whereas a heteroaromatic ring system must have at least one heteroatom as ring atom. This heteroatom may be present as a ring atom of a nonaromatic heterocyclic ring or as a ring atom of an aromatic heterocyclic ring.

In accordance with the above definitions, any aryl group is covered by the term “aromatic ring system”, and any heteroaryl group is covered by the term “heteroaromatic ring system”.

An aromatic ring system having 6 to 40 aromatic ring atoms or a heteroaromatic ring system having 5 to 40 aromatic ring atoms is especially understood to mean groups derived from the groups mentioned above under aryl groups and heteroaryl groups, and from biphenyl, terphenyl, quaterphenyl, fluorene, spirobifluorene, dihydrophenanthrene, dihydropyrene, tetrahydropyrene, indenofluorene, truxene, isotruxene, spirotruxene, spiroisotruxene, indenocarbazole, or from combinations of these groups.

In the context of the present invention, a straight-chain alkyl group having 1 to 20 carbon atoms and a branched or cyclic alkyl group having 3 to 20 carbon atoms and an alkenyl or alkynyl group having 2 to 40 carbon atoms in which individual hydrogen atoms or CH₂ groups may also be substituted by the groups mentioned above in the definition of the radicals are preferably understood to mean the methyl, ethyl, n-propyl, i-propyl, n-butyl, i-butyl, s-butyl, t-butyl, 2-methylbutyl, n-pentyl, s-pentyl, cyclopentyl, neopentyl, n-hexyl, cyclohexyl, neohexyl, n-heptyl, cycloheptyl, n-octyl, cyclooctyl, 2-ethylhexyl, trifluoromethyl, pentafluoroethyl, 2,2,2-trifluoroethyl, ethenyl, propenyl, butenyl, pentenyl, cyclopentenyl, hexenyl, cyclohexenyl, heptenyl, cycloheptenyl, octenyl, cyclooctenyl, ethynyl, propynyl, butynyl, pentynyl, hexynyl or octynyl radicals.

An alkoxy or thioalkyl group having 1 to 20 carbon atoms in which individual hydrogen atoms or CH₂ groups may also be substituted by the groups mentioned above in the definition of the radicals is preferably understood to mean methoxy, trifluoromethoxy, ethoxy, n-propoxy, i-propoxy, n-butoxy, i-butoxy, s-butoxy, t-butoxy, n-pentoxy, s-pentoxy, 2-methylbutoxy, n-hexoxy, cyclohexyloxy, n-heptoxy, cycloheptyloxy, n-octyloxy, cyclooctyloxy, 2-ethylhexyloxy, pentafluoroethoxy, 2,2,2-trifluoroethoxy, methylthio, ethylthio, n-propylthio, i-propylthio, n-butylthio, i-butylthio, s-butylthio, t-butylthio, n-pentylthio, s-pentylthio, n-hexylthio, cyclohexylthio, n-heptylthio, cycloheptylthio, n-octylthio, cyclooctylthio, 2-ethylhexylthio, trifluoromethylthio, pentafluoroethylthio, 2,2,2-trifluoroethylthio, ethenylthio, propenylthio, butenylthio, pentenylthio, cyclopentenylthio, hexenylthio, cyclohexenylthio, heptenylthio, cycloheptenylthio, octenylthio, cyclooctenylthio, ethynylthio, propynylthio, butynylthio, pentynylthio, hexynylthio, heptynylthio or octynylthio.

The wording that two or more radicals together may form a ring, in the context of the present application, shall be understood to mean, inter alia, that the two radicals are joined to one another by a chemical bond. In addition, however, the abovementioned wording shall also be understood to mean that, if one of the two radicals is hydrogen, the second radical binds to the position to which the hydrogen atom was bonded, forming a ring.

The compound of the formula (I) is preferably a monoamine. This means that the compound has only a single triarylamino group, and preferably only one amino group. A triarylamino group here means a unit having three groups selected from aromatic and heteroaromatic groups bonded to the amino nitrogen atom.
Z¹ is preferably CR¹, where the unit

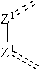

may be replaced by a unit selected from units of the formulae (Z-1) to (Z-6), where the dotted bonds in the units each correspond to the bonds in the unit

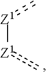

and where the variables Z¹ in the unit

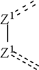

in these cases are C.

In a preferred embodiment, all Z¹ groups are CR¹, and

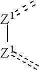

units cannot be replaced by units of the formulae (Z-1) to (Z-6).
Ar⁰ preferably conforms to one of the following formulae:

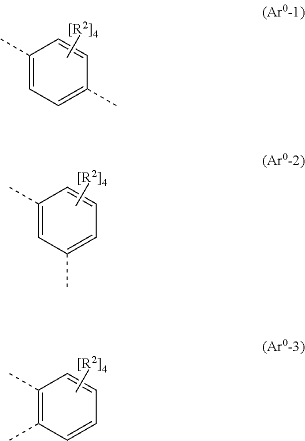

where the dotted lines represent the bonds to the rest of the formula; more preferably to the formula (Ar⁰-1).

In a preferred embodiment, m is 0 or 1.

In a preferred embodiment, n is 0 or 1. In a particularly preferred embodiment, n is 1. In an alternative particularly preferred embodiment, n is 0.

In a preferred embodiment, at least one index selected from indices m and n is greater than 0; more preferably, at least one index selected from indices m and n is 1, and the other index selected from indices m and n is 0 or 1, preferably 0.

Ar^{L} is preferably an aromatic ring system which has 6 to 25 aromatic ring atoms and is substituted by R³ radicals, is more preferably selected from phenyl, biphenyl, naphthyl or fluorenyl, each substituted by R³ radicals, and is most preferably selected from phenyl substituted by R³ radicals.

Preferably, Ar^{L} is the same or different at each instance and is selected from groups of the following formulae:

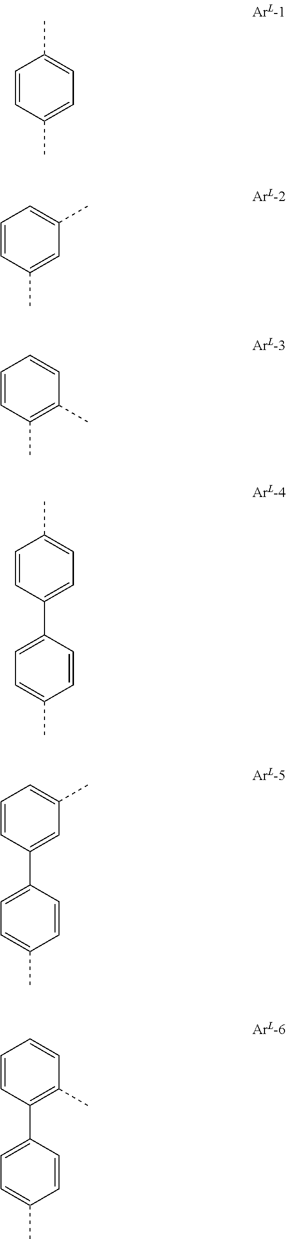

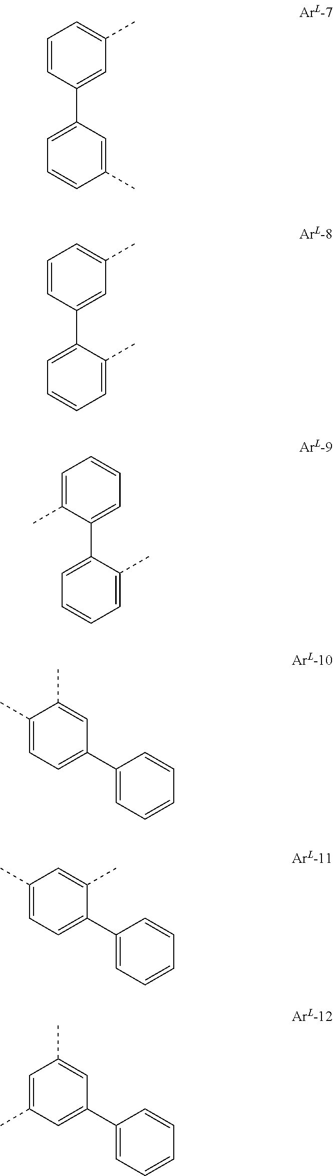

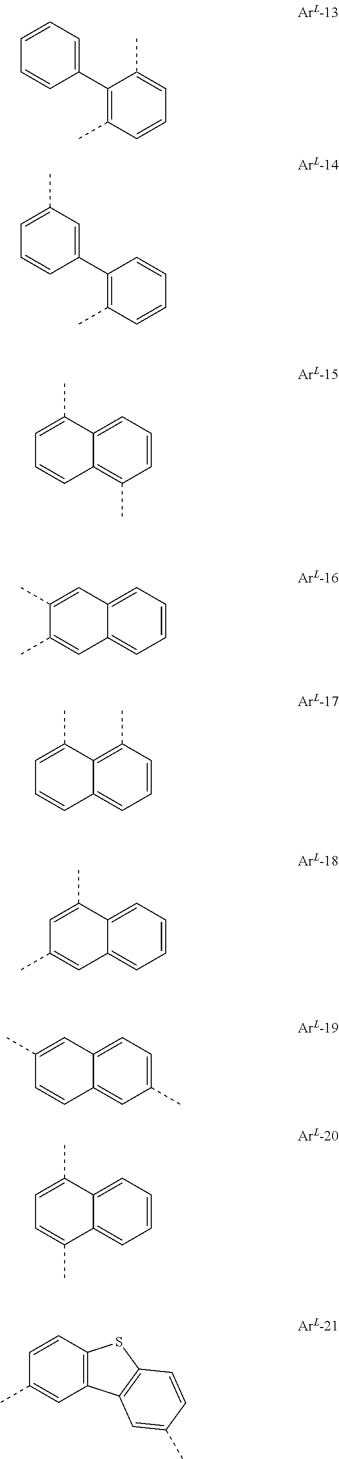

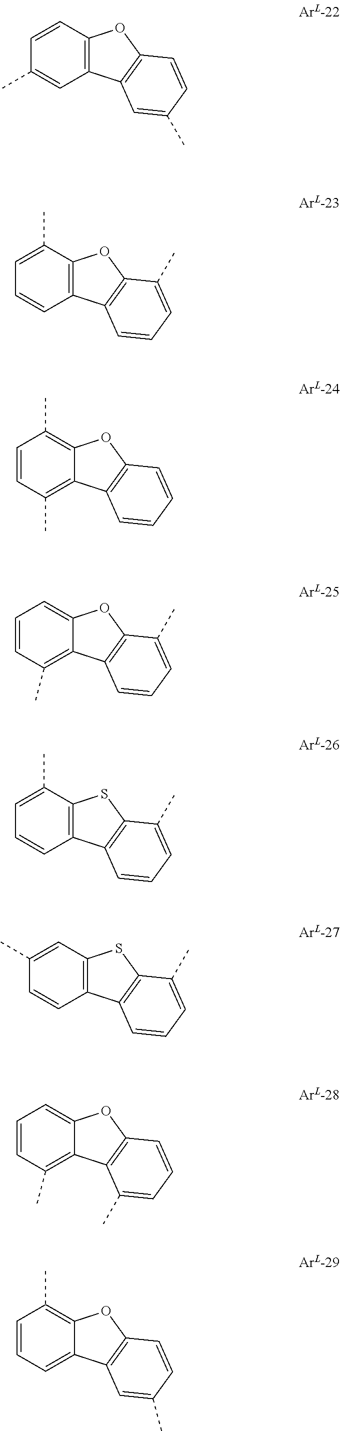

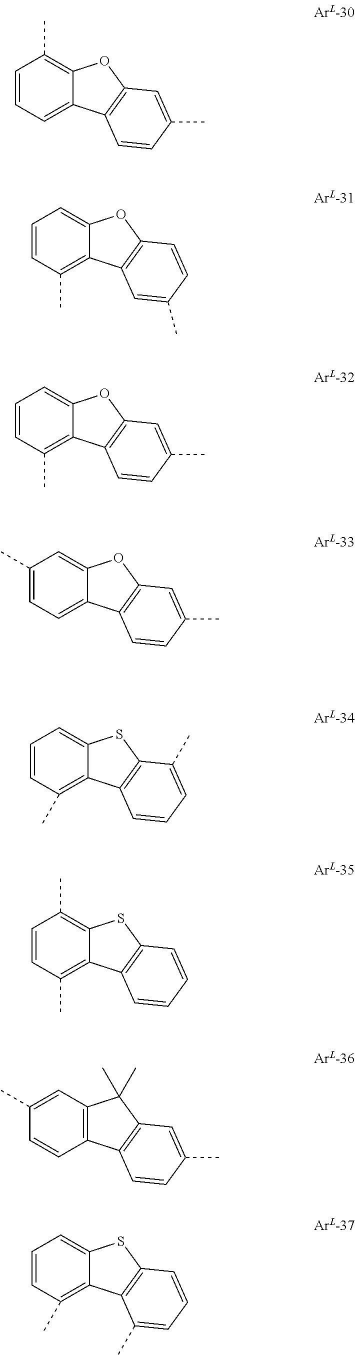

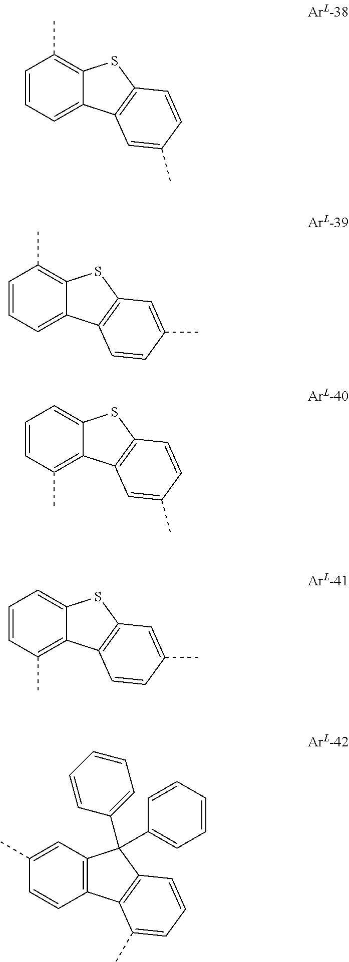

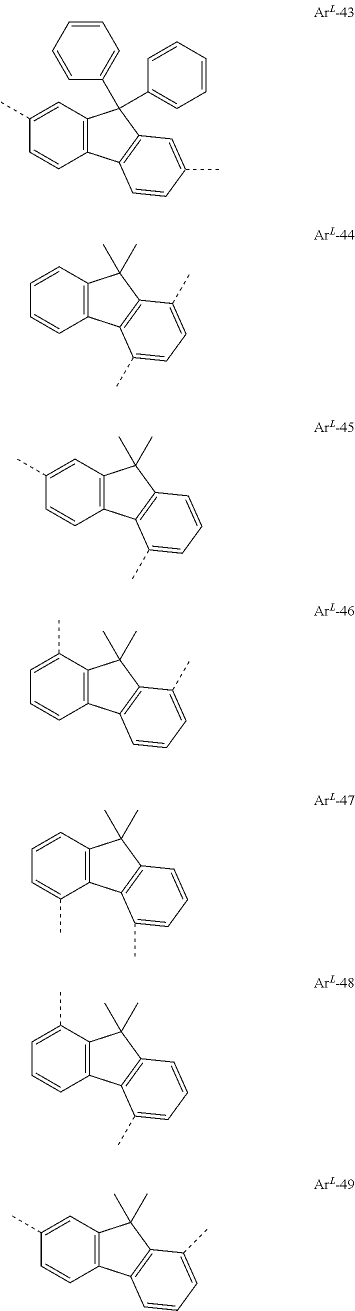

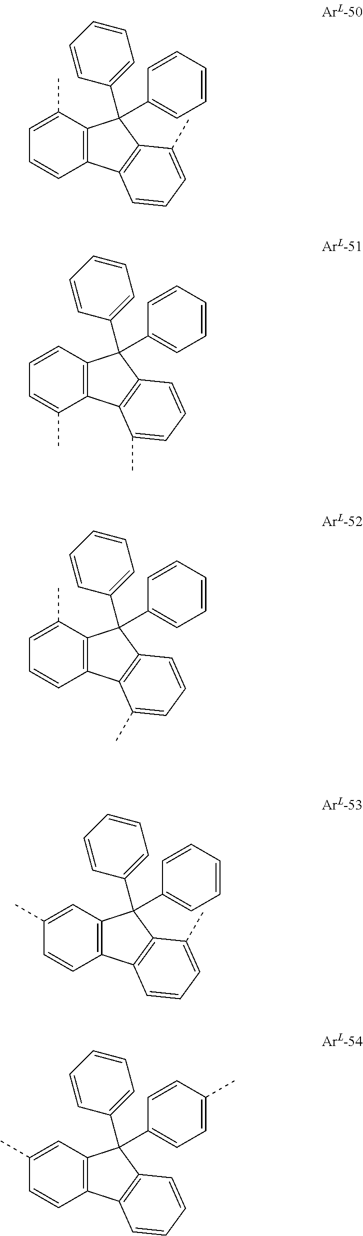

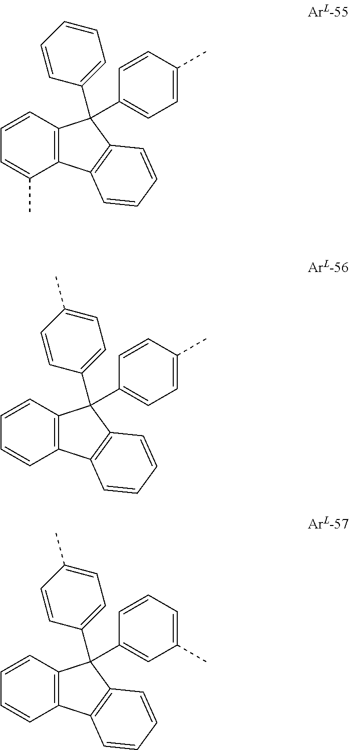

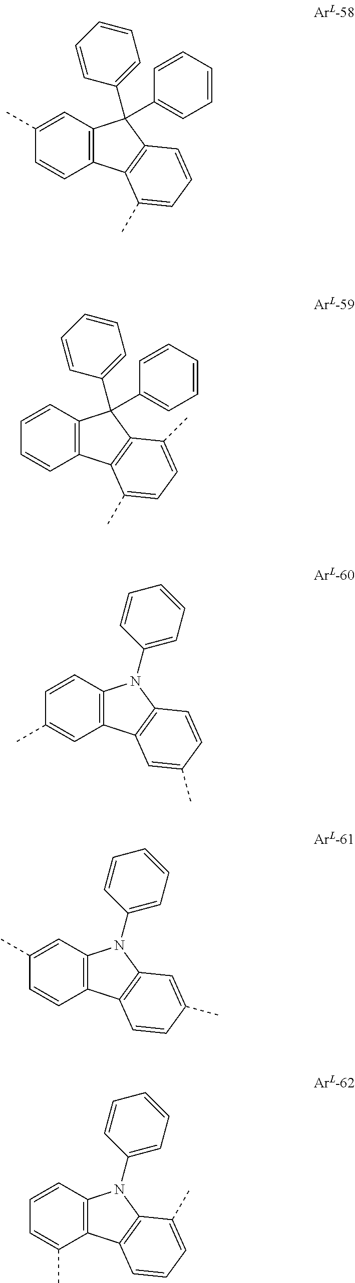

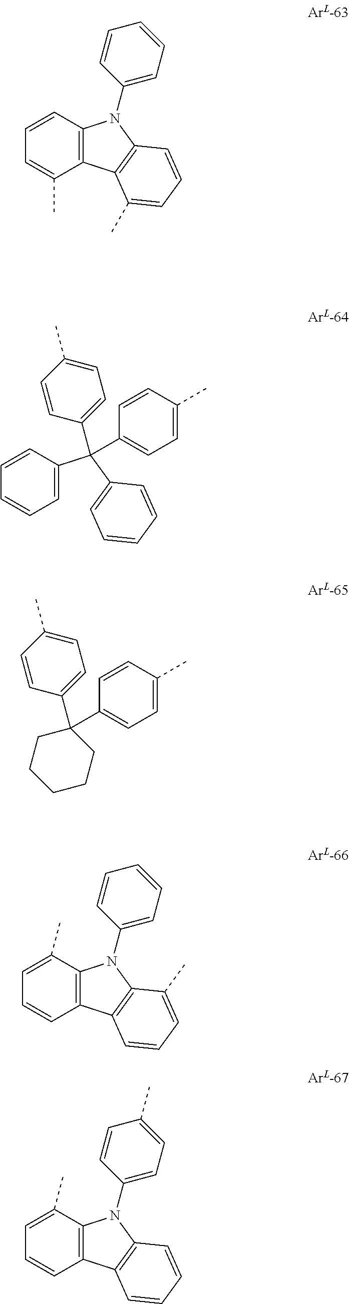

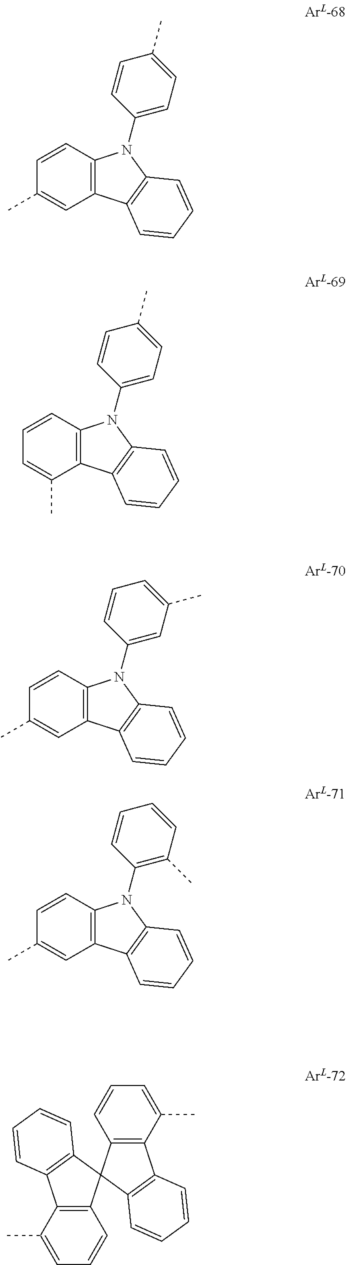

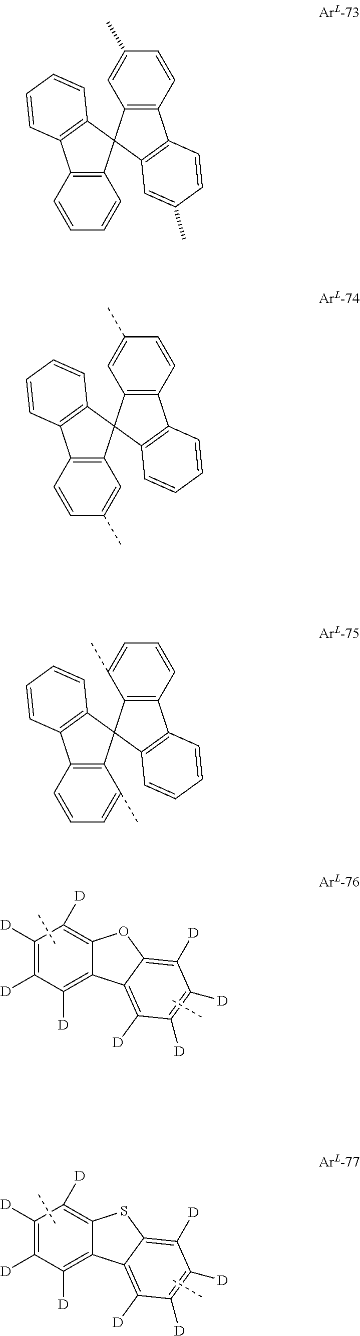

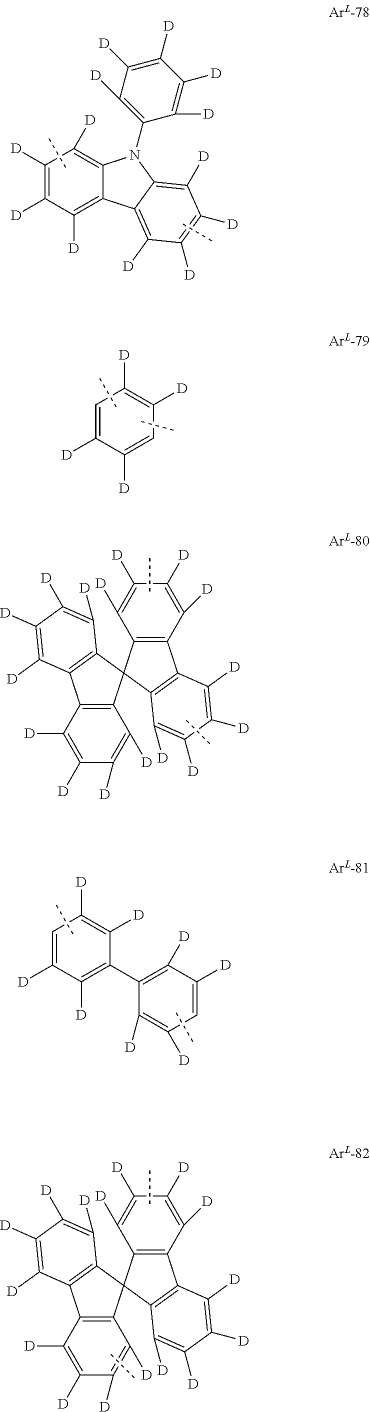

where the dotted lines represent the bonds to the rest of the formula, and where the groups at the positions shown as being unsubstituted may each bear an R³ radical.

Among the formulae listed above, particular preference is given to the formulae Ar^{L}-1, Ar^{L}-2, Ar^{L}-3 and Ar^{L}-4.

In a preferred embodiment, k is 0. In an alternative preferred embodiment, k is 1.

Preferred Ar¹ and Ar² groups are the same or different at each instance and are selected from monovalent groups derived from benzene, biphenyl, terphenyl, quaterphenyl, naphthalene, phenanthrene, fluorene, especially 9,9′-dimethylfluorene and 9,9′-diphenylfluorene, benzofluorene, spirobifluorene, indenofluorene, indenocarbazole, dibenzofuran, dibenzothiophene, benzocarbazole, carbazole, benzofuran, benzothiophene, indole, quinoline, pyridine, pyrimidine, pyrazine, pyridazine and triazine, where each of the monovalent groups is substituted by R⁴ radicals. Preference is given to Ar¹ and Ar² groups that are the same or different at each instance and are selected from combinations of 2 to 4 groups derived from benzene, biphenyl, terphenyl, quaterphenyl, naphthalene, phenanthrene, fluorene, especially 9,9′-dimethylfluorene and 9,9′-diphenylfluorene, benzofluorene, spirobifluorene, indenofluorene, indenocarbazole, dibenzofuran, dibenzothiophene, benzocarbazole, carbazole, benzofuran, benzothiophene, indole, quinoline, pyridine, pyrimidine, pyrazine, pyridazine and triazine, where each of the monovalent groups is substituted by R⁴ radicals.

Particularly preferred Ar¹ and Ar² groups are the same or different at each instance and are selected from benzene, biphenyl, terphenyl, quaterphenyl, naphthyl, phenanthrenyl, fluorenyl, especially 9,9′-dimethylfluorenyl and 9,9′-diphenylfluorenyl, benzofluorenyl, spirobifluorenyl, indenofluorenyl, indenocarbazolyl, dibenzofuranyl, dibenzothiophenyl, carbazolyl, benzofuranyl, benzothiophenyl, benzofused dibenzofuranyl, benzofused dibenzothiophenyl, and phenyl substituted by a group selected from naphthyl, phenanthrenyl, fluorenyl, spirobifluorenyl, dibenzofuranyl, dibenzothiophenyl, carbazolyl, pyridyl, pyrimidyl and triazinyl, where the groups are each substituted by R⁴ radicals.

Ar¹ and Ar² are preferably the same or different at each instance and are selected from the following formulae:

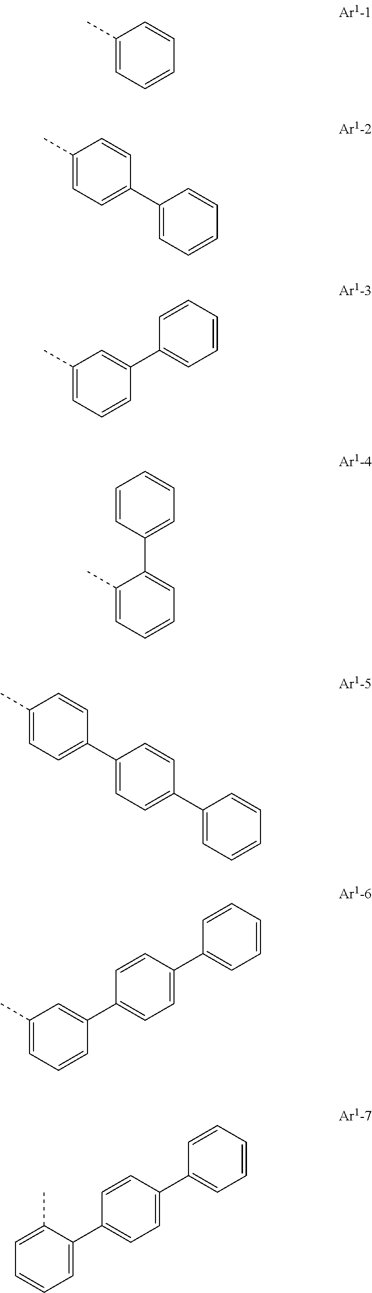

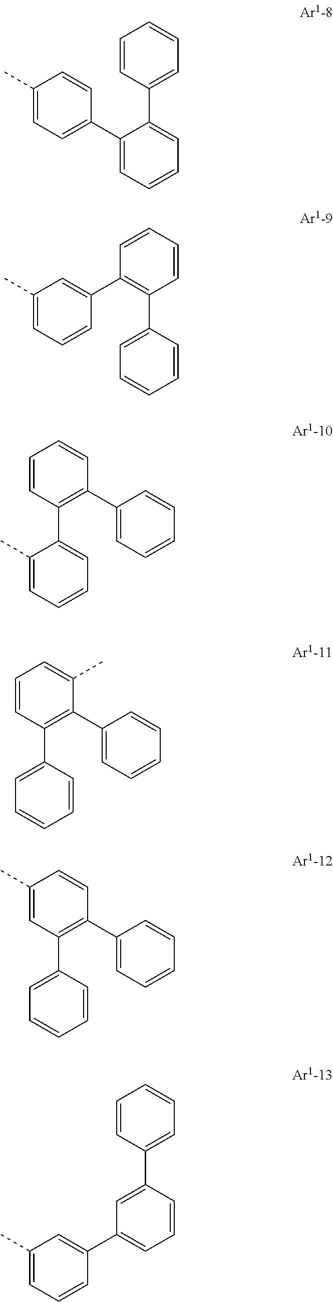

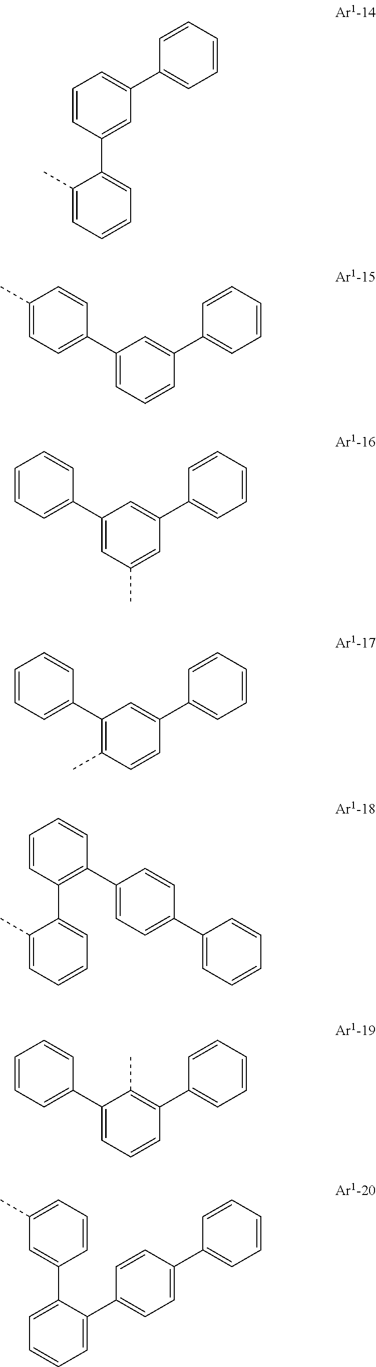

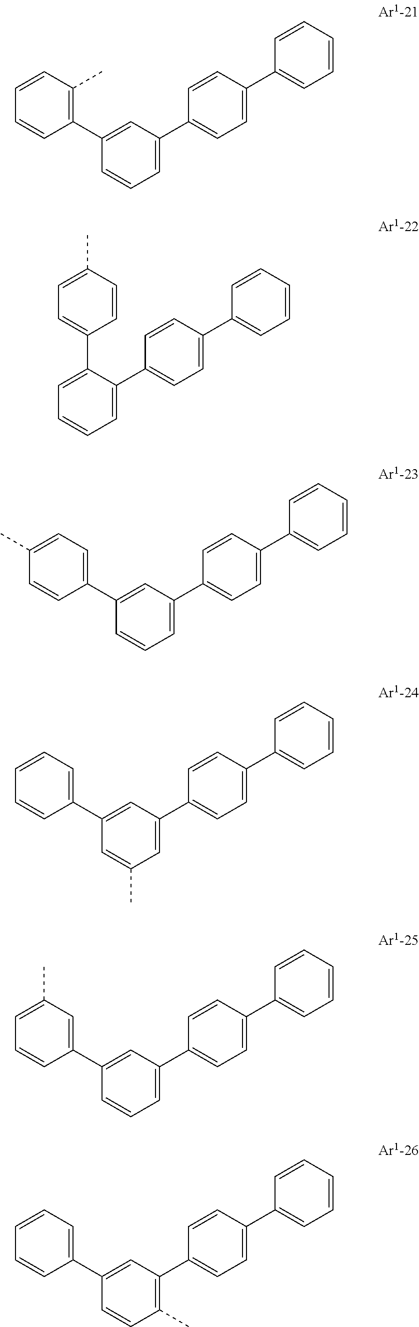

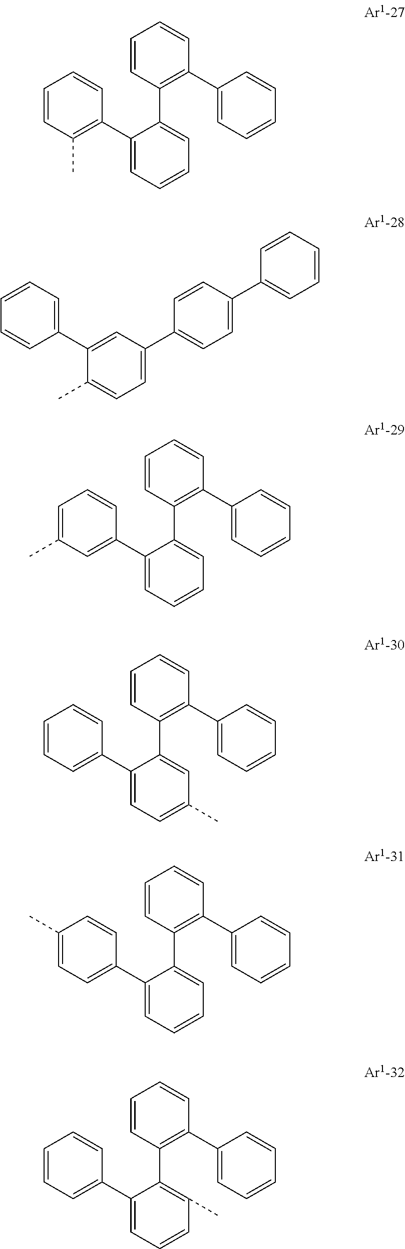

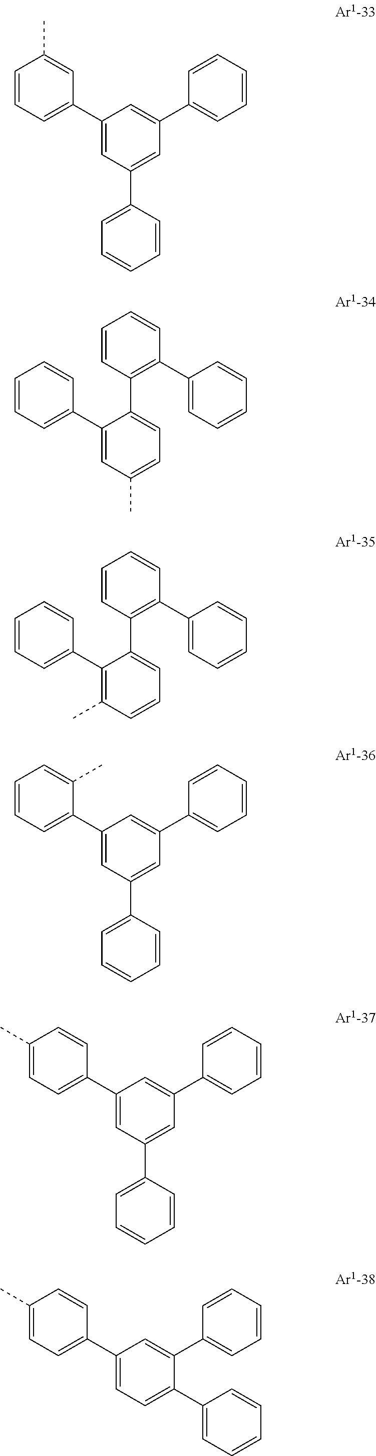

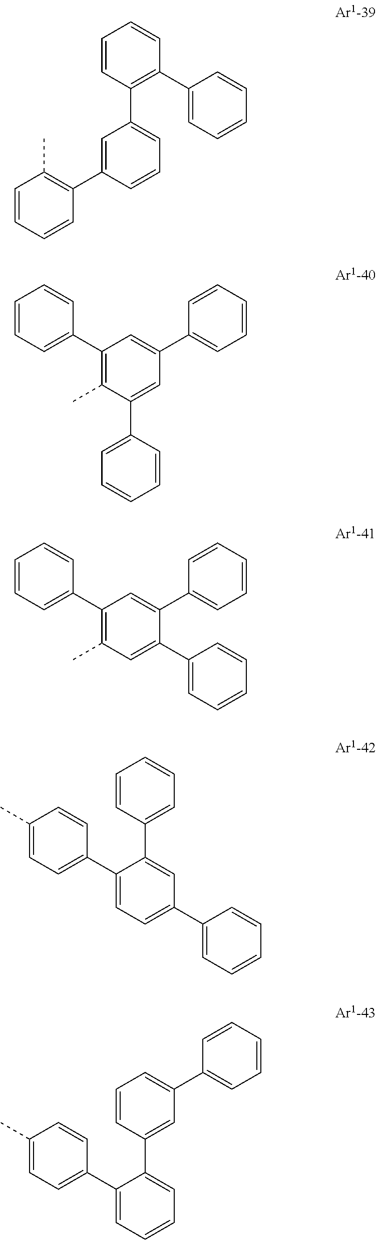

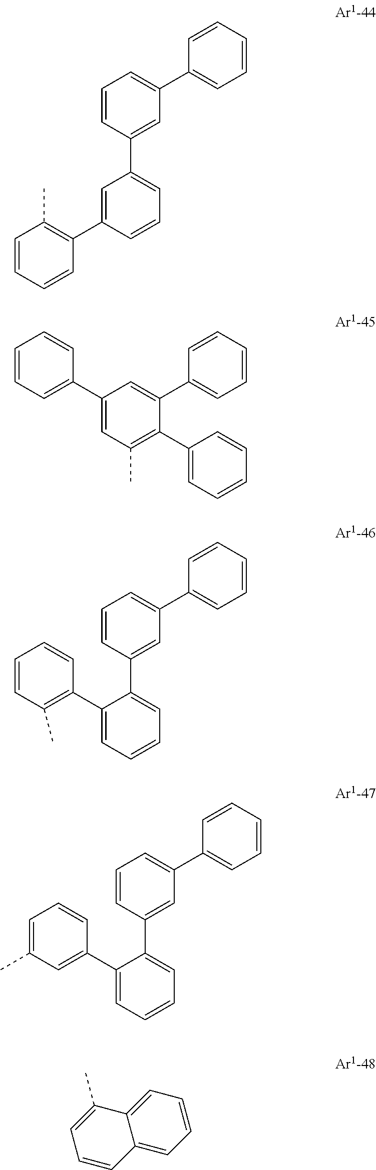

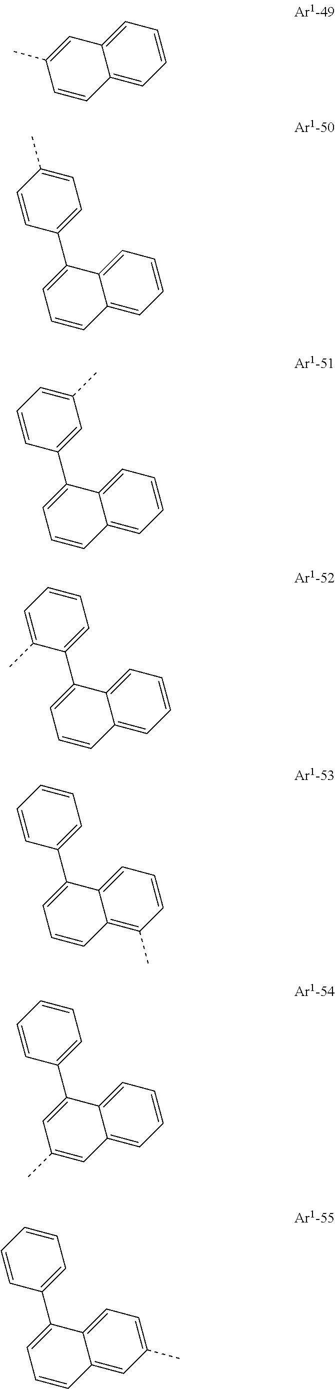

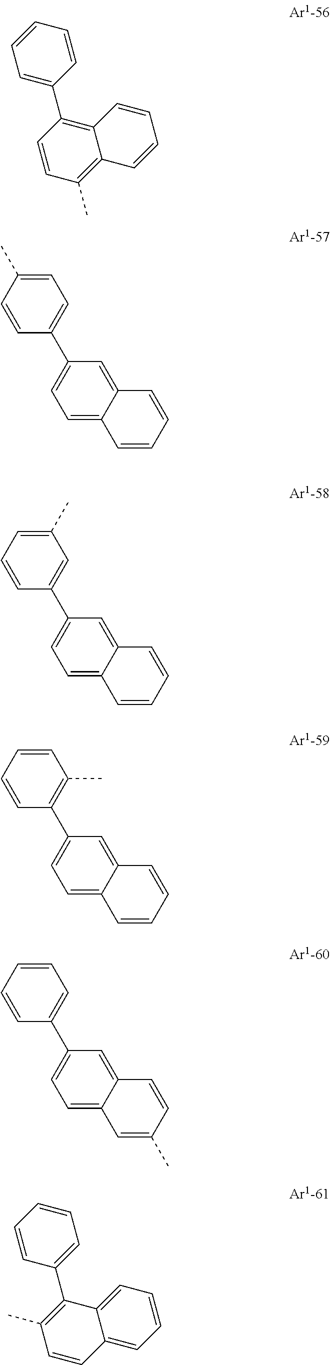

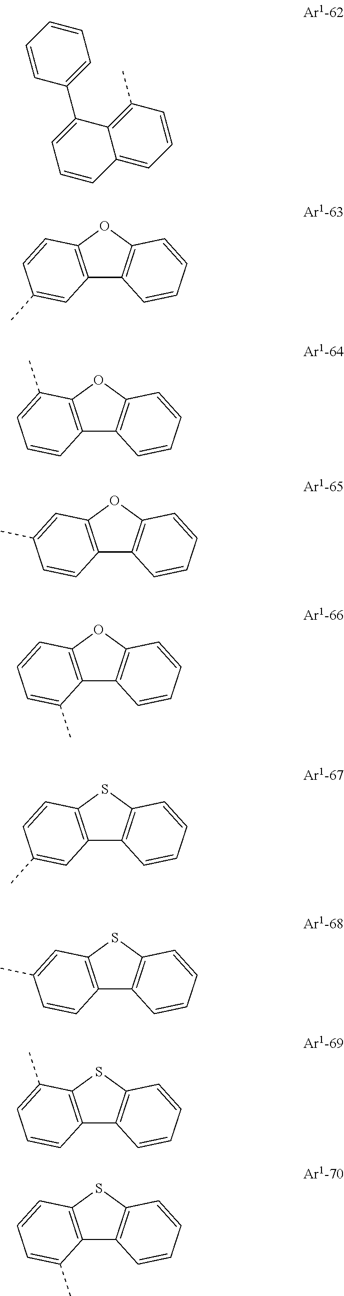

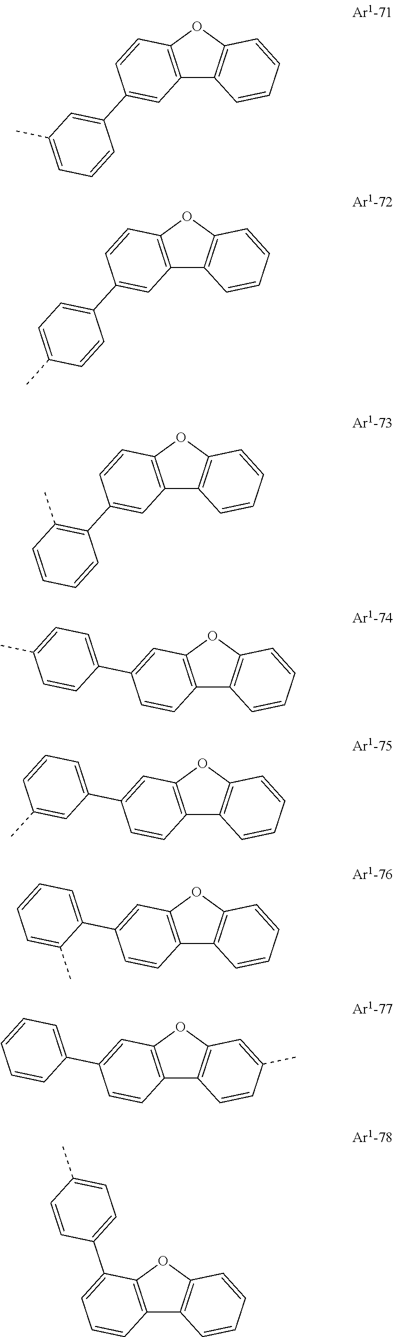

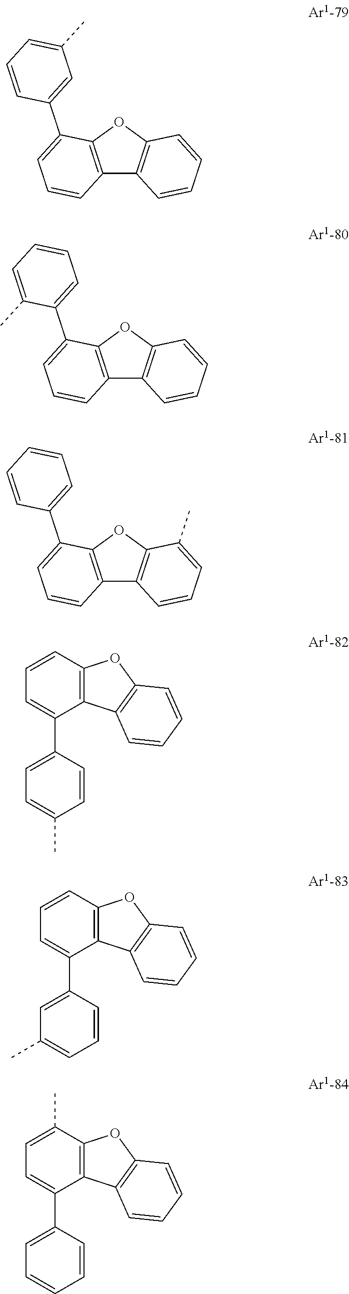

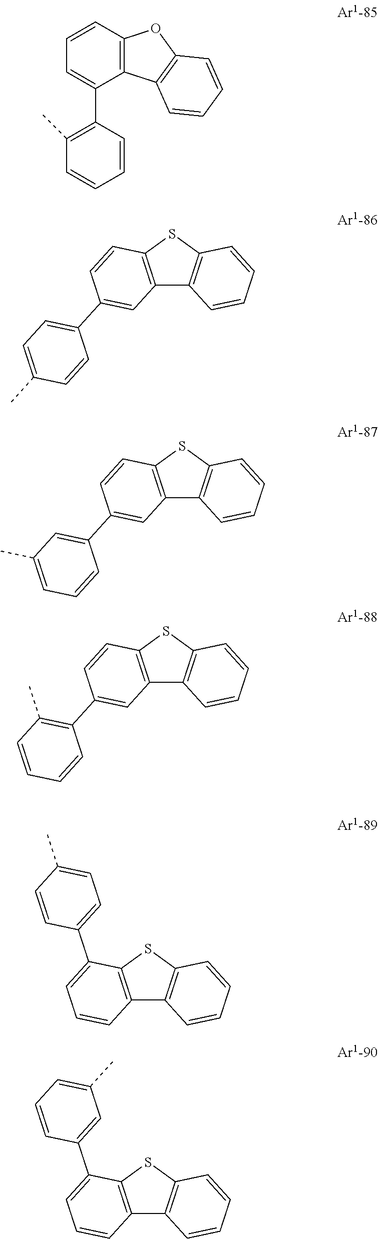

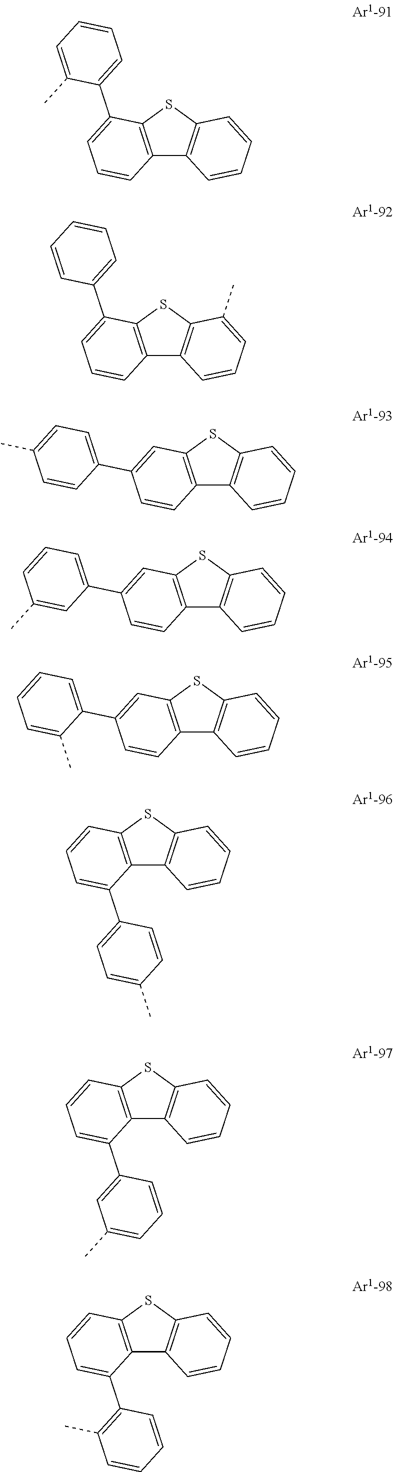

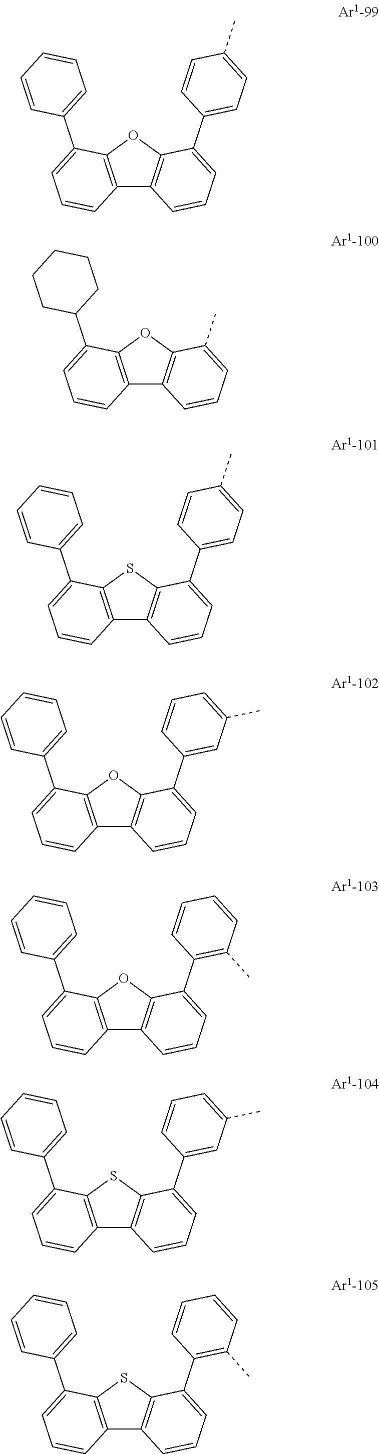

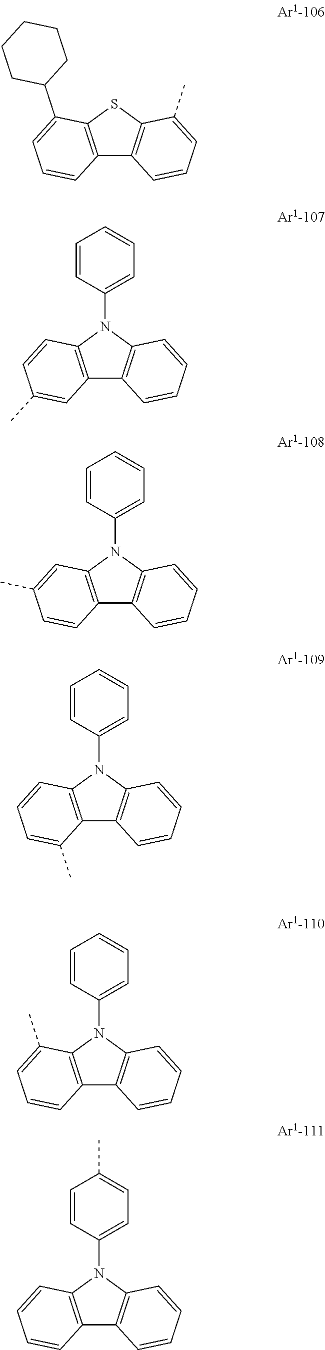

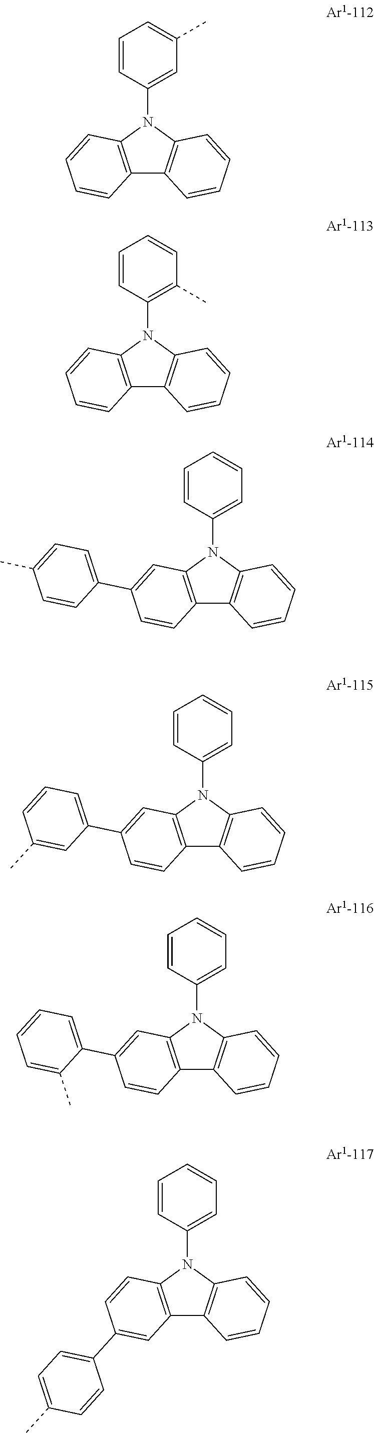

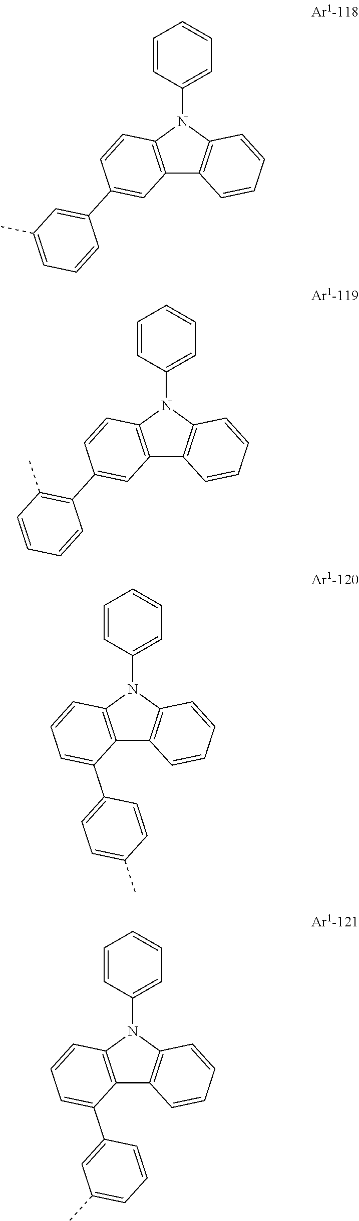

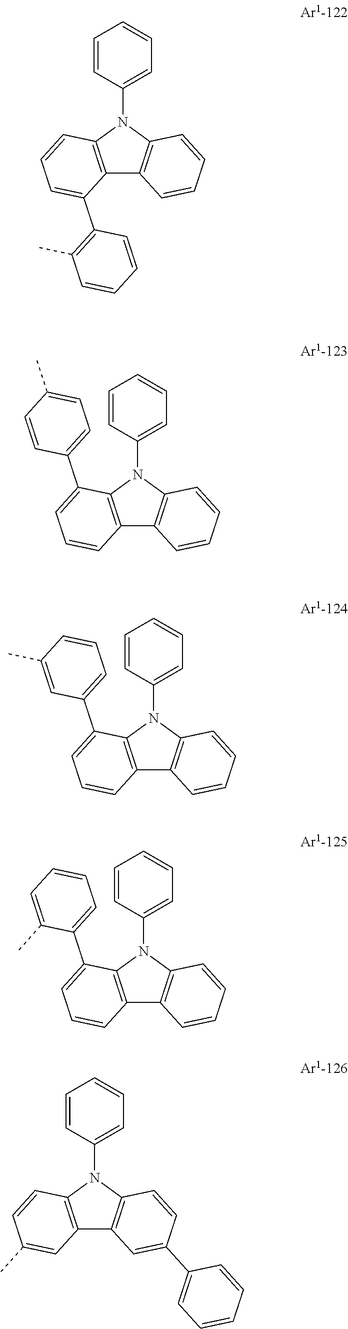

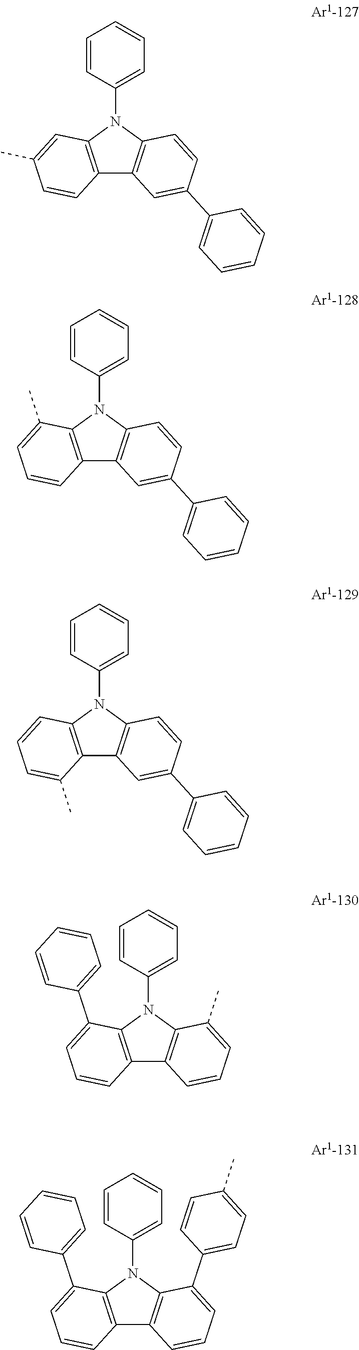

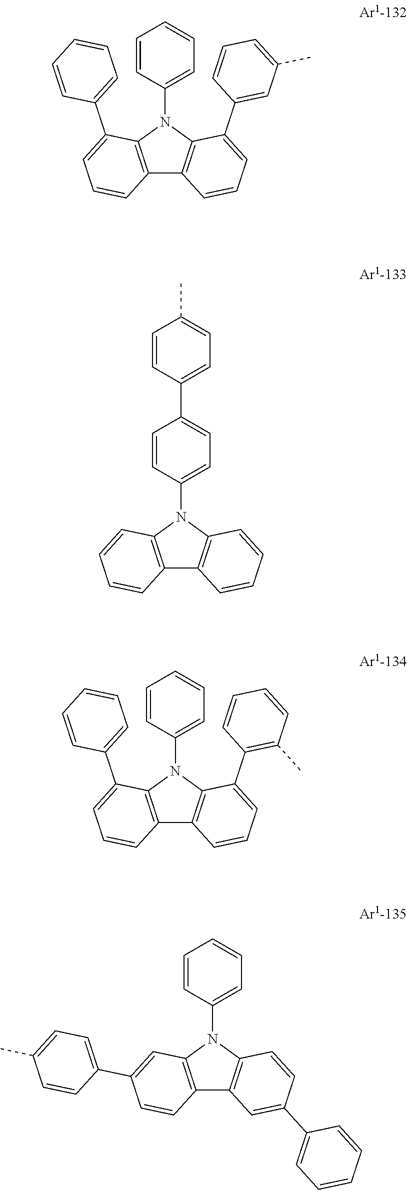

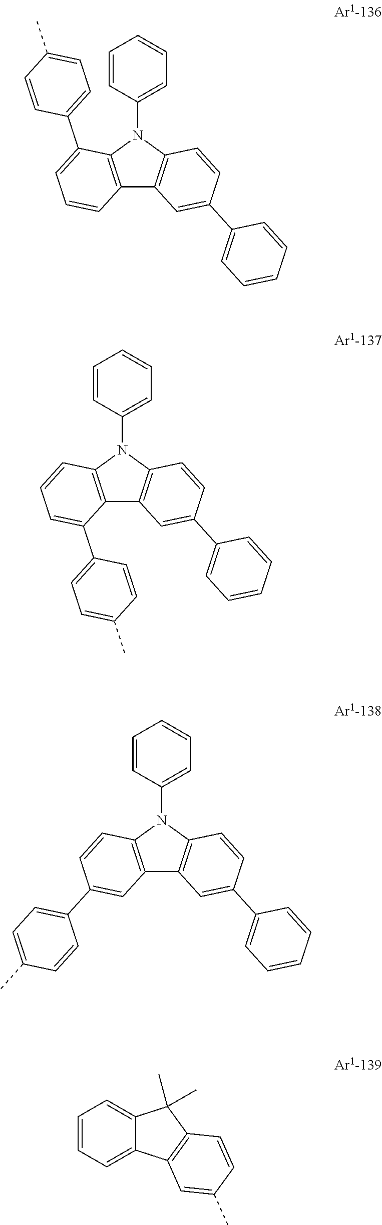

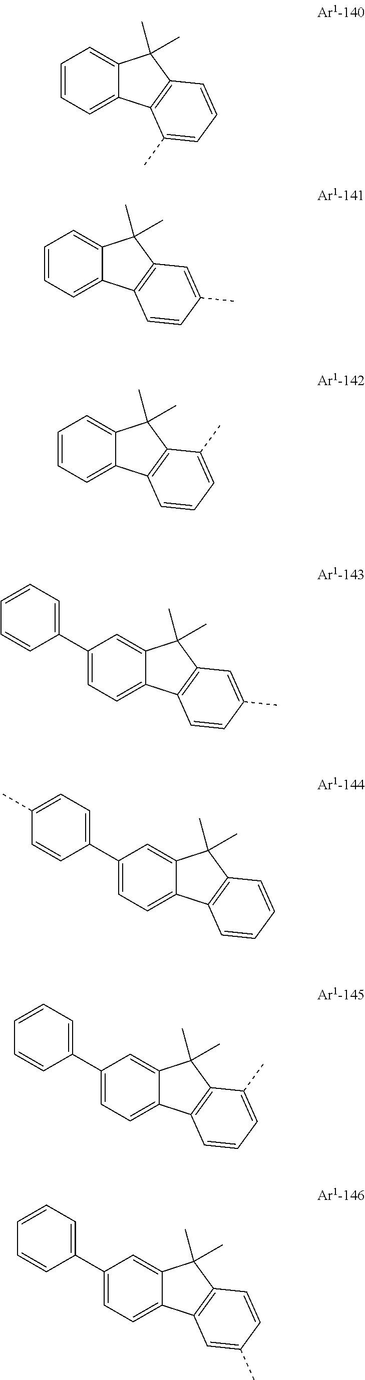

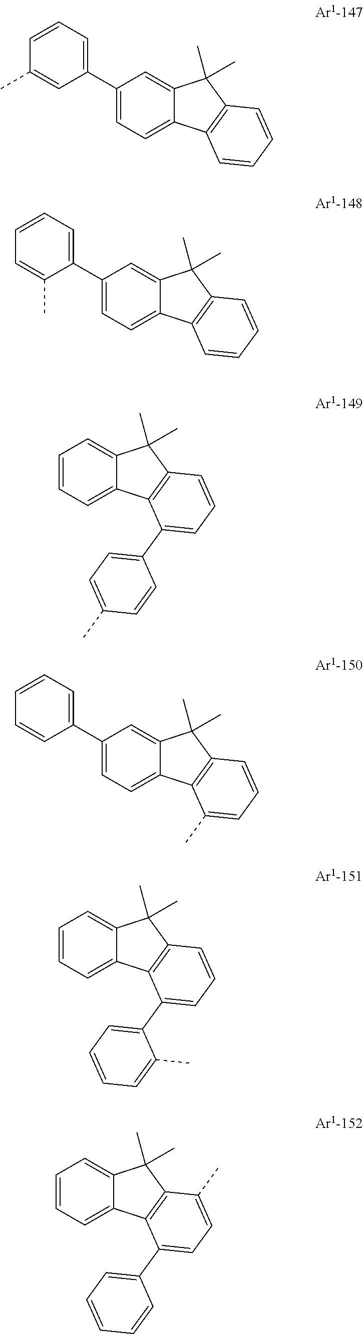

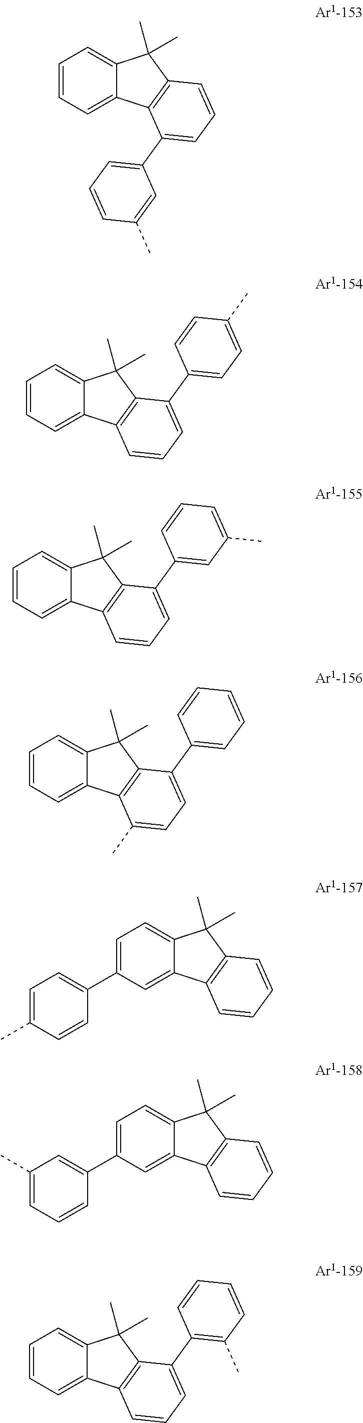

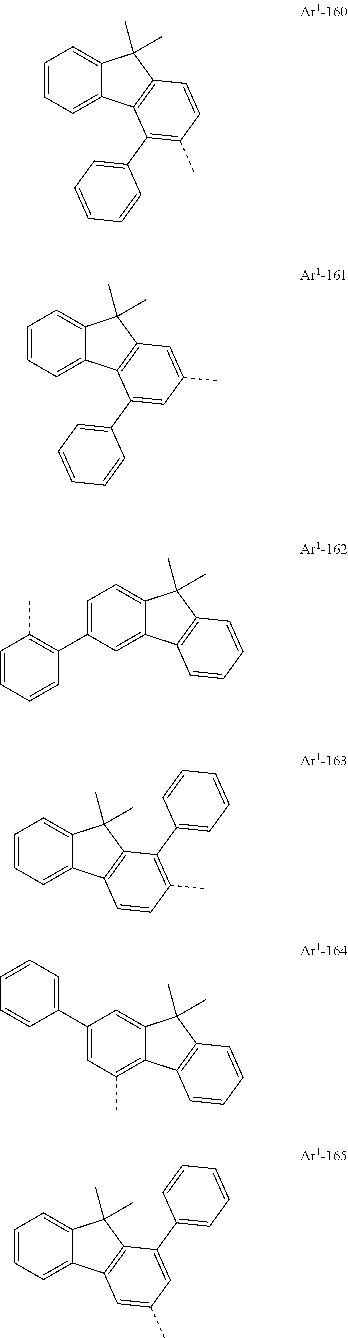

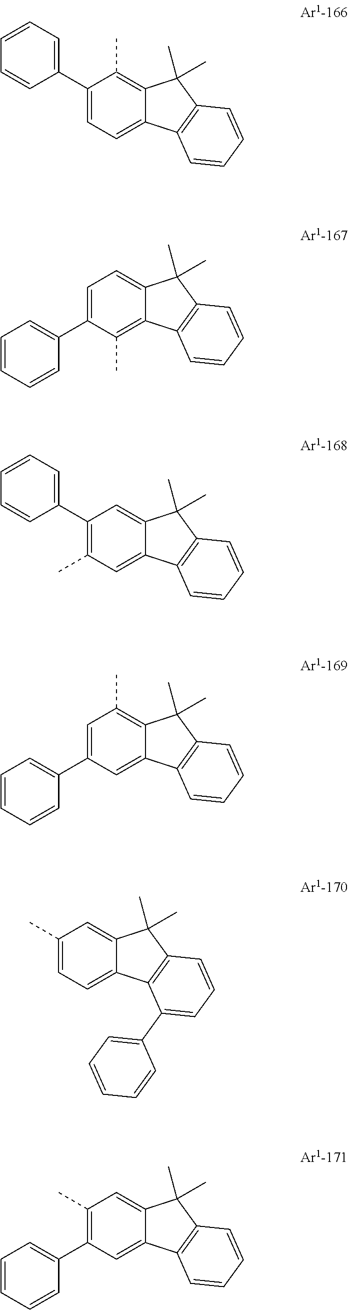

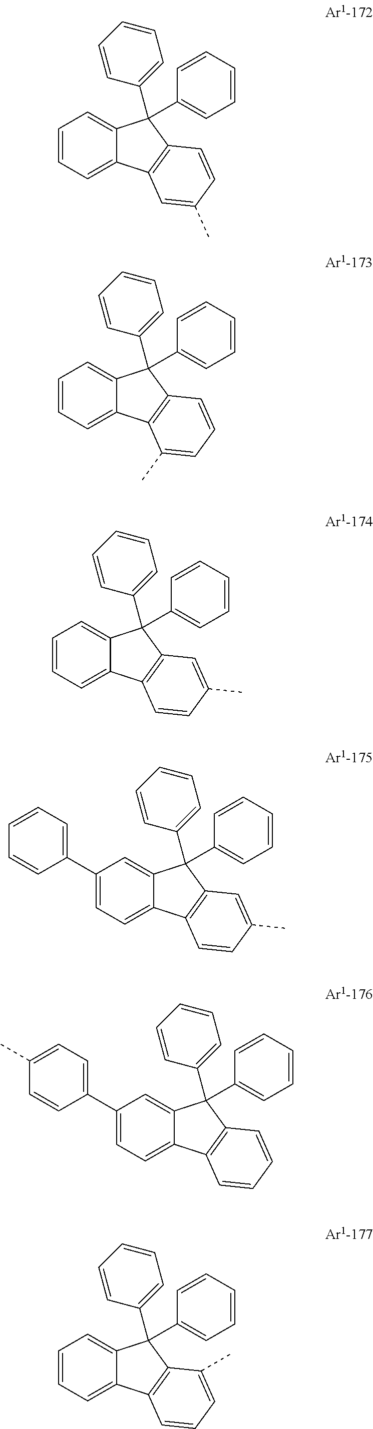

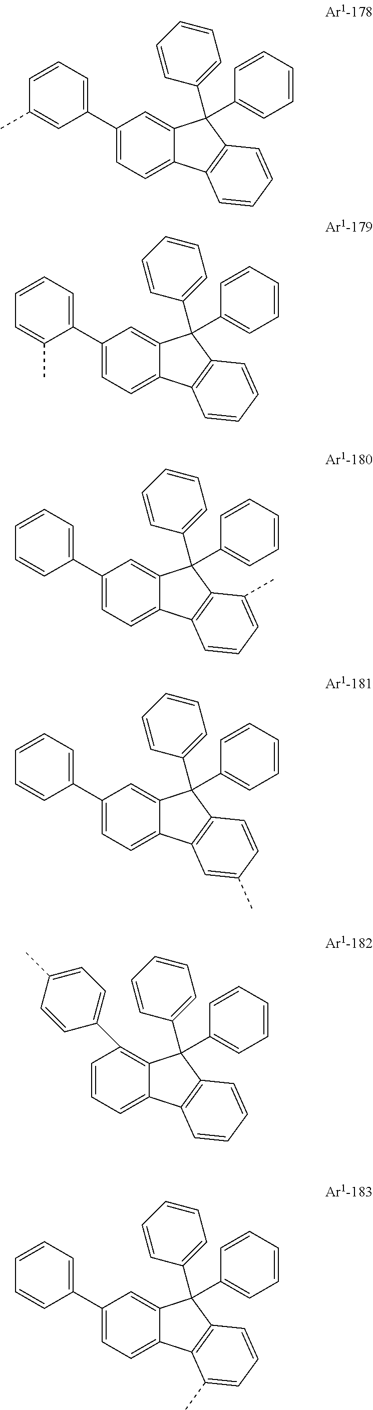

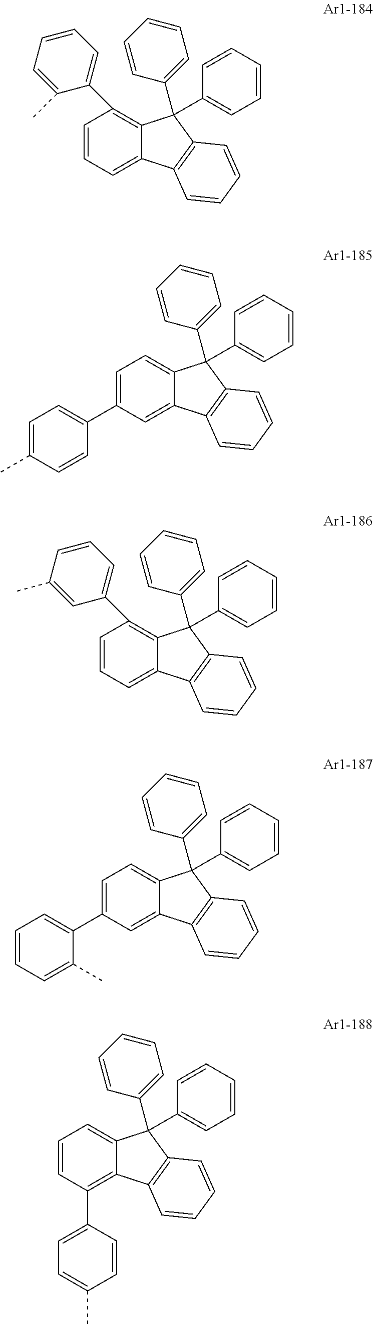

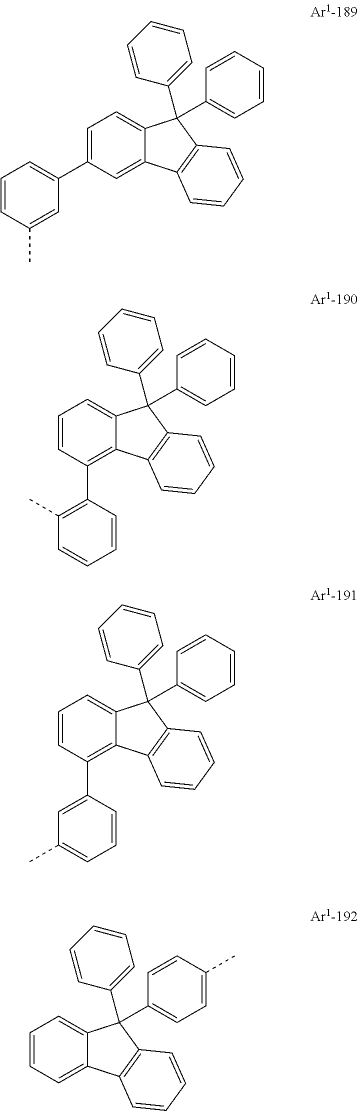

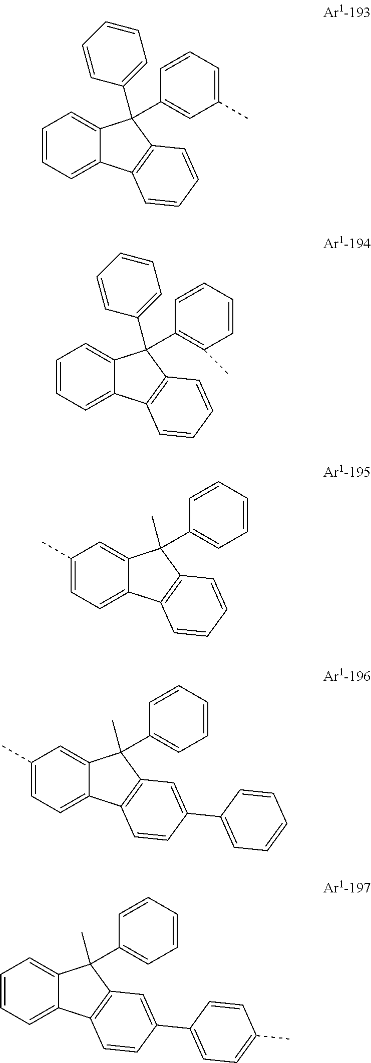

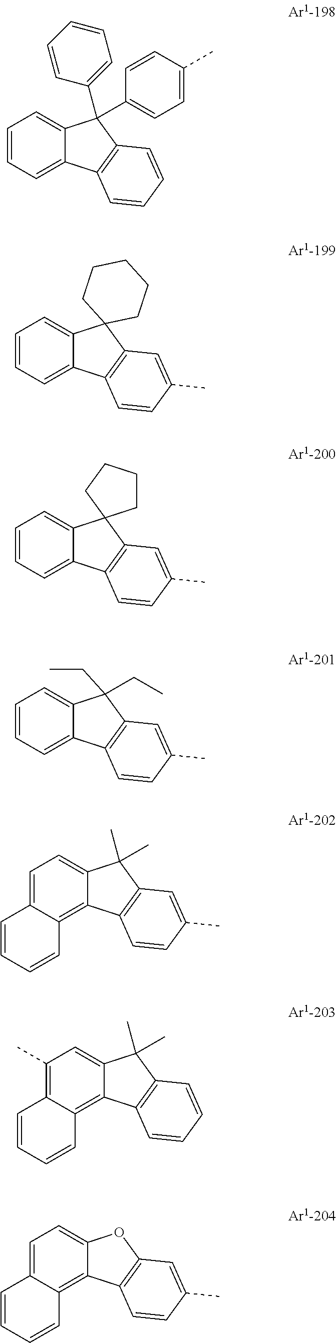

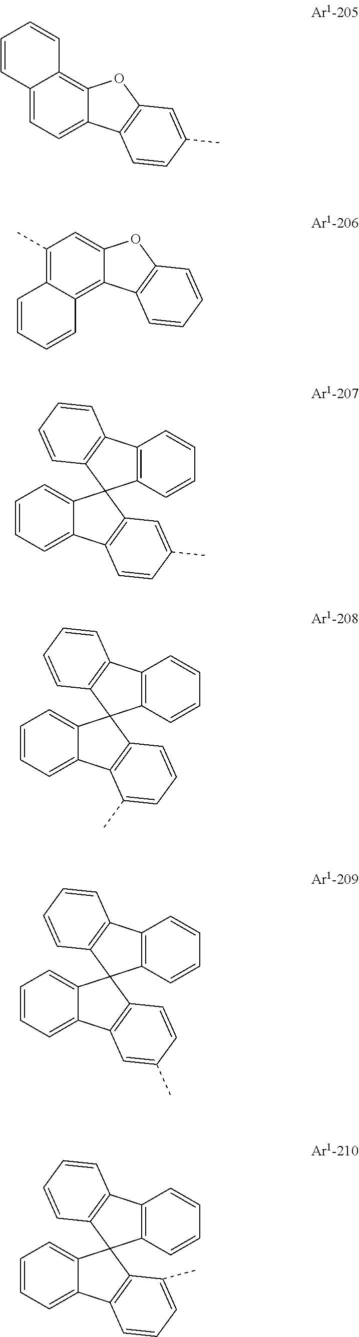

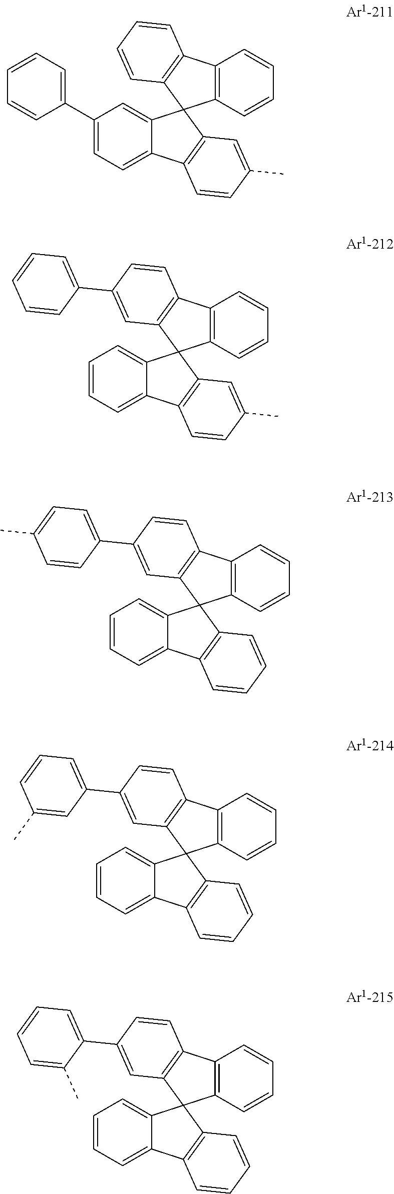

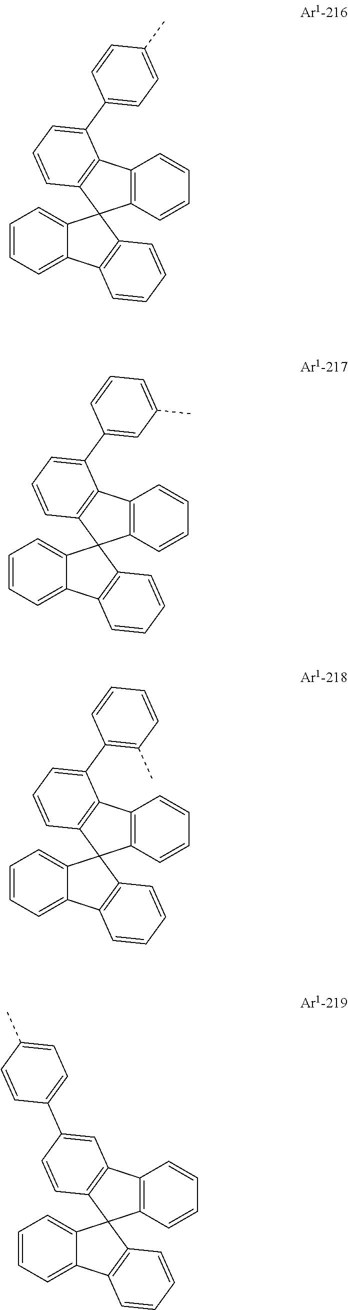

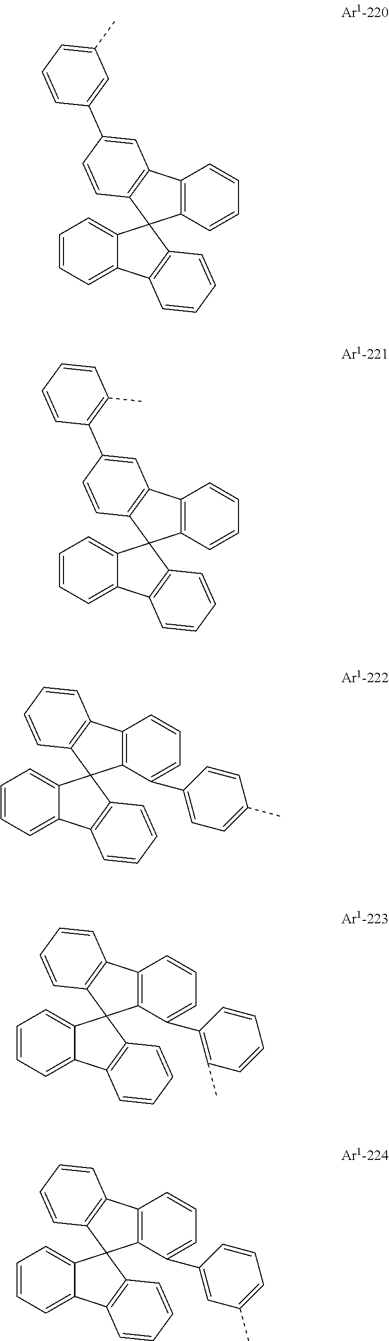

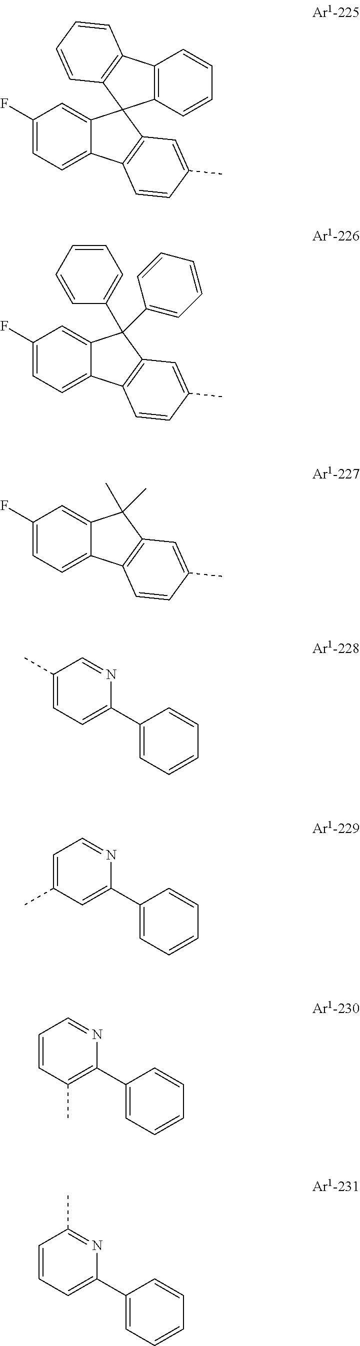

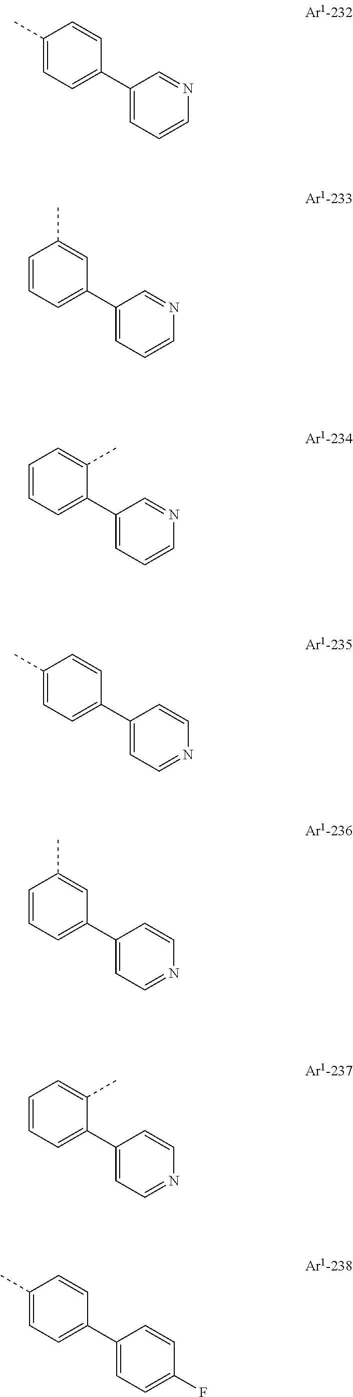

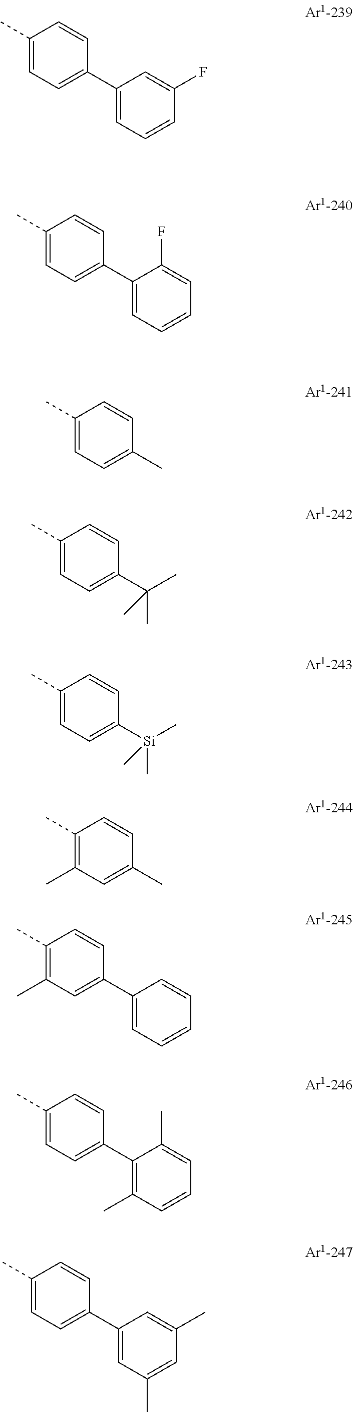

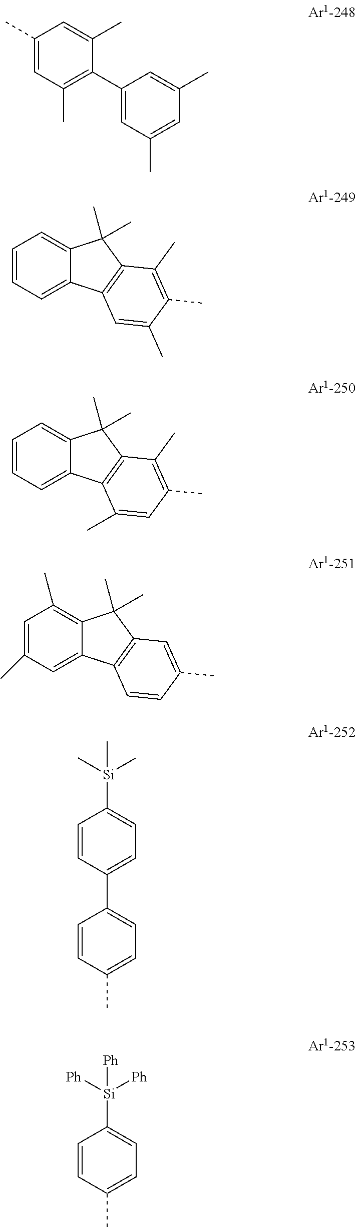

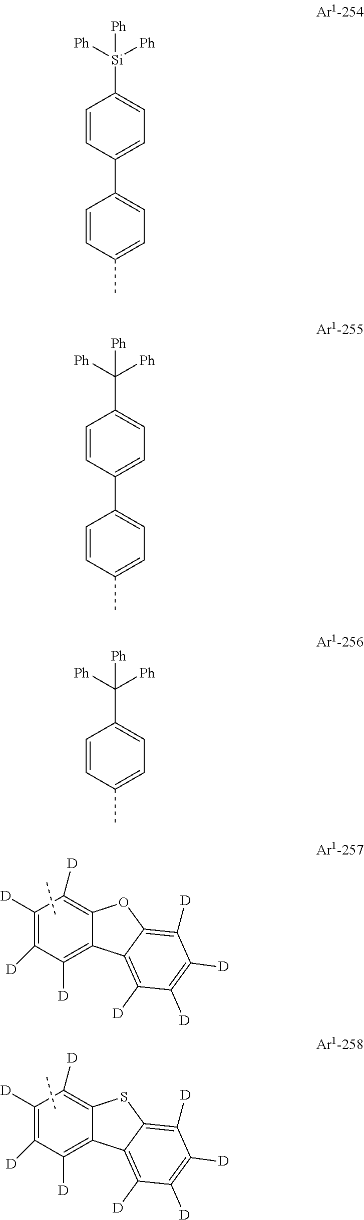

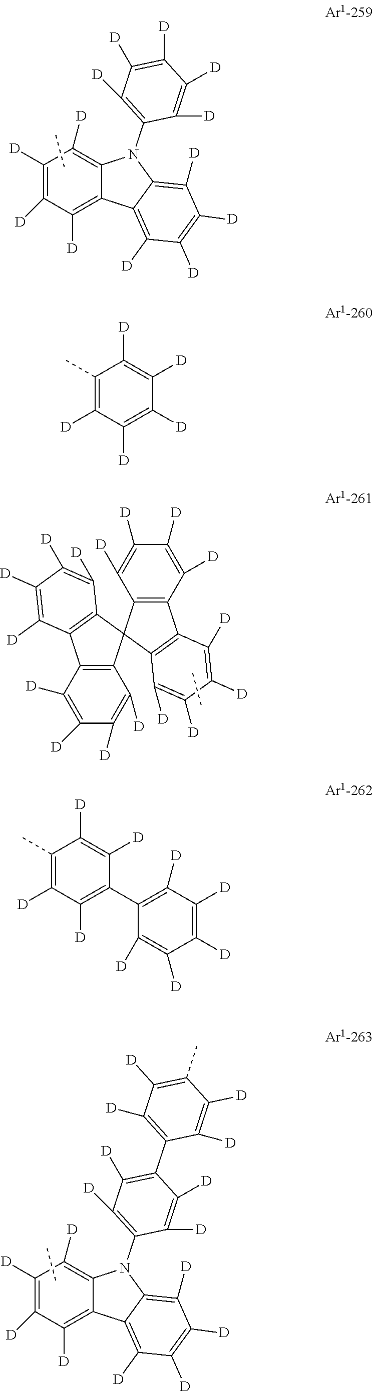

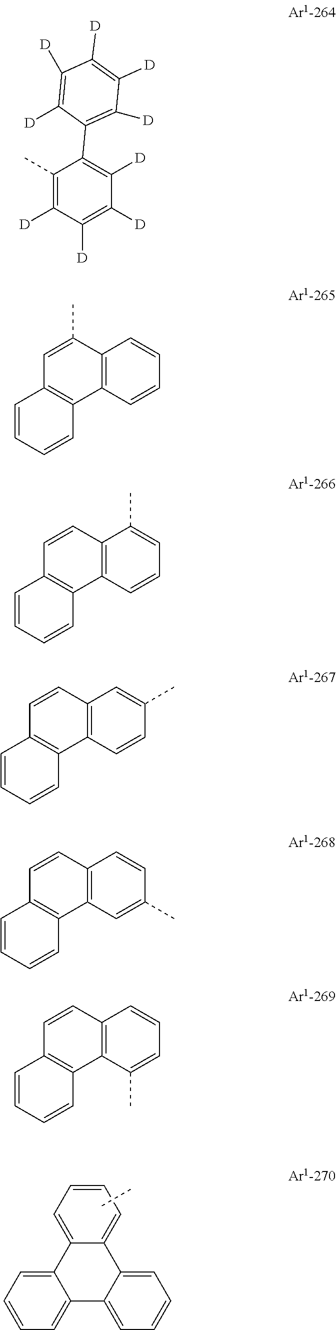

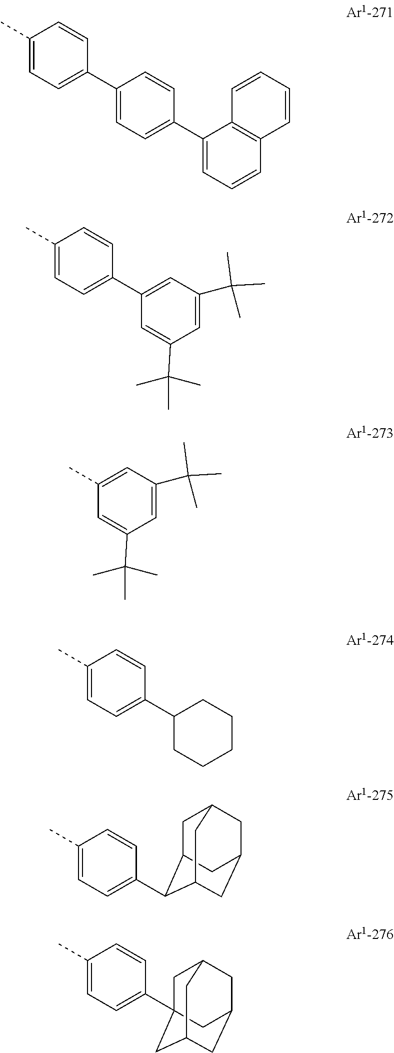

where the dotted line represents the bond to the nitrogen atom and where the groups at the position is shown as unsubstituted may be substituted by R⁴ radicals, and preferably have only H in the positions shown as unsubstituted.

Preferably, Ar³ and Ar⁴ are the same or different at each instance and are selected from aromatic ring systems which have 6 to 40 aromatic ring atoms and are substituted by R⁵ radicals.

Particularly preferred Ar³ and Ar⁴ groups are the same or different at each instance and are selected from phenyl, biphenyl, terphenyl, quaterphenyl, naphthyl, phenanthrenyl, fluoranthenyl, fluorenyl, especially 9,9′-dimethylfluorenyl and 9,9′-diphenylfluorenyl, benzofluorenyl, spirobifluorenyl, indenofluorenyl, indenocarbazolyl, dibenzofuranyl, dibenzothiophenyl, carbazolyl, benzofuranyl, benzothiophenyl, benzofused dibenzofuranyl, benzofused dibenzothiophenyl, and phenyl substituted by a group selected from naphthyl, fluorenyl, spirobifluorenyl, dibenzofuranyl, dibenzothiophenyl, carbazolyl, pyridyl, pyrimidyl and triazinyl, where the groups are each substituted by R⁵ radicals. Most preferably, Ar³ and Ar⁴ are the same or different at each instance and are selected from phenyl, biphenyl, naphthyl, phenanthrenyl, fluoranthenyl, triphenylenyl and fluorenyl, especially 9,9′-dimethylfluorenyl and 9,9′-diphenylfluorenyl, where the groups are each substituted by R⁵ radicals.

Ar³ and Ar⁴ are preferably the same or different at each instance and are selected from the formulae (Ar¹-1) to (Ar¹-276) shown above, where the dotted line represents the bond to the nitrogen atom and where the groups at the positions shown as unsubstituted may be substituted by R⁵ radicals, and preferably have only H in the positions shown as unsubstituted.

The at least one group selected from Ar³ and Ar⁴ groups that must be selected from aryl groups which have 10 to 20 aromatic ring atoms and are substituted by R⁵ radicals is preferably selected from naphthyl, anthracenyl, phenanthrenyl, fluoranthenyl, triphenylenyl, each of which are substituted by R⁵ radicals, more preferably selected from naphthyl, phenanthrenyl and fluoranthenyl, substituted by R⁵ radicals, most preferably naphthyl substituted by R⁵ radicals. R⁵ radicals in these cases are preferably selected from H, D, alkyl groups having 1 to 20 carbon atoms, and aromatic ring systems having 6 to 20 aromatic ring atoms, which are substituted by R⁶ radicals, where R⁶ in this case is preferably H or D.

More preferably, the at least one group selected from Ar³ and Ar⁴ groups that must be selected from aryl groups which have 10 to 20 aromatic ring atoms and are substituted by R⁵ radicals is selected from the groups of the following formulae:

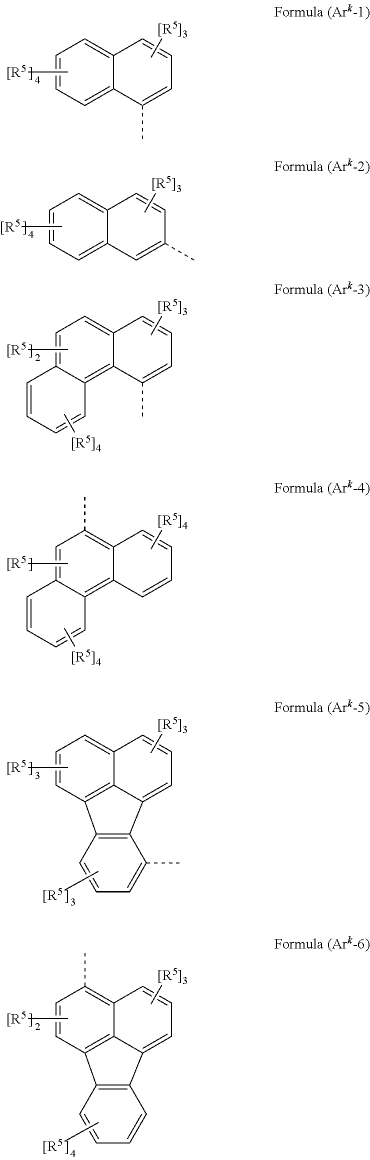

where the variables that occur are as defined above and preferably conform to their preferred embodiments, and where the dotted line is the bond of the Ar³ or Ar⁴ group to the rest of the compound.

When Ar³ and Ar⁴ are each naphthyl substituted by R⁵ radicals, preferably at least one index selected from indices m and n is greater than 0.

It is preferable, in the case that n=0, that Ar⁴ is not selected from naphthyl substituted by R⁵ radicals and phenanthrenyl substituted by R⁵ radicals. It is preferable, in the case that k=0, that Ar⁴ is not selected from naphthyl substituted by R⁵ radicals and phenanthrenyl substituted by R⁵ radicals. In addition, it is particularly preferable that Ar⁴ is not selected from naphthyl substituted by R⁵ radicals and phenanthrenyl substituted by R⁵ radicals.

It is preferable, in the case that n=0, that Ar⁴ is selected from phenyl, anthracenyl, fluoranthenyl and triphenylenyl, each substituted by R⁵ radicals, especially that Ar⁴ is phenyl substituted by R⁵ radicals. It is preferable, in the case that k=0, that Ar⁴ is selected from phenyl, anthracenyl, fluoranthenyl and triphenylenyl, each substituted by R⁵ radicals, especially that Ar⁴ is phenyl substituted by R⁵ radicals. In addition, it is particularly preferable that Ar⁴ is selected from phenyl, anthracenyl, fluoranthenyl and triphenylenyl, each substituted by R⁵ radicals, especially that Ar⁴ is phenyl substituted by R⁵ radicals.

R¹ is preferably the same or different at each instance and is selected from H, D, F, CN, Si(R⁶)₃, N(R⁶)₂, straight-chain alkyl groups having 1 to 20 carbon atoms, and branched or cyclic alkyl groups having 3 to 20 carbon atoms; where the alkyl groups mentioned are each substituted by R⁶ radicals and where one or more CH₂ groups in said alkyl groups may be replaced by —C≡C—, —R⁶C≡CR⁶—, Si(R⁶)₂, C═O, C≡NR⁶, —NR⁶—, —O—, —S—, —C(═O)O— or —C(═O)NR⁶—. R¹ is more preferably the same or different at each instance and is selected from H, D, F, CN, straight-chain alkyl groups having 1 to 20 carbon atoms, branched or cyclic alkyl groups having 3 to 20 carbon atoms; where said alkyl groups are each substituted by R⁶ radicals. Even more preferably, R¹ is the same or different at each instance and is selected from H and D.

R², R³ and R⁵ are preferably the same or different at each instance and are selected from H, D, F, CN, Si(R⁶)₃, N(R⁶)₂, straight-chain alkyl groups having 1 to 20 carbon atoms, branched or cyclic alkyl groups having 3 to 20 carbon atoms, and aromatic ring systems having 6 to 40 aromatic ring atoms, where the alkyl groups mentioned and the aromatic ring systems mentioned are each substituted by R⁶ radicals; and where one or more CH₂ groups in the alkyl groups mentioned may be replaced by —C≡C—, —R⁶C≡CR⁶—, Si(R⁶)₂, C═O, C═NR⁶, —NR⁶—, —O—, —S—, —C(═O)O— or —C(═O)NR⁶—. R², R³ and R⁵ are more preferably the same or different at each instance and are selected from H, D, F, CN, straight-chain alkyl groups having 1 to 20 carbon atoms, branched or cyclic alkyl groups having 3 to 20 carbon atoms, and aromatic ring systems having 6 to 40 aromatic ring atoms; where said alkyl groups and said aromatic ring systems are each substituted by R⁶ radicals.

R⁴ is preferably the same or different at each instance and is selected from H, D, F, CN, Si(R⁶)₃, straight-chain alkyl groups having 1 to 20 carbon atoms, branched or cyclic alkyl groups having 3 to 20 carbon atoms, aromatic ring systems having 6 to 40 aromatic ring atoms and heteroaromatic ring systems having 5 to 40 aromatic ring atoms; where the alkyl groups mentioned, the aromatic ring systems mentioned and the heteroaromatic ring systems mentioned are each substituted by R⁶ radicals; and where one or more CH₂ groups in the alkyl groups mentioned may be replaced by —C≡C—, —R⁶C≡CR⁶—, Si(R⁶)₂, C═O, C═NR⁶, —NR⁶—, —O—, —S—, —C(═O)O— or —C(═O)NR⁶—. R⁴ is more preferably the same or different at each instance and is selected from H, D, F, CN, straight-chain alkyl groups having 1 to 20 carbon atoms, branched or cyclic alkyl groups having 3 to 20 carbon atoms, aromatic ring systems having 6 to 40 aromatic ring atoms, and heteroaromatic ring systems having 5 to 40 aromatic ring atoms; where said alkyl groups, said aromatic ring systems and said heteroaromatic ring systems are each substituted by R⁶ radicals.

Preferably, R⁶ is the same or different at each instance and is selected from H, D, F, CN, Si(R⁷)₃, N(R⁷)₂, straight-chain alkyl groups having 1 to 20 carbon atoms, branched or cyclic alkyl groups having 3 to 20 carbon atoms, aromatic ring systems having 6 to 40 aromatic ring atoms, and heteroaromatic ring systems having 5 to 40 aromatic ring atoms; where said alkyl groups, said aromatic ring systems and said heteroaromatic ring systems are each substituted by R⁷ radicals; and where one or more CH₂ groups in said alkyl groups may be replaced by —C≡C—, —R⁷C≡CR⁷—, Si(R⁷)₂, C═O, C═NR⁷, —NR⁷—, —O—, —S—, —C(═O)O— or —C(═O)NR⁷—. R⁶ is more preferably the same or different at each instance and is selected from H, D, F, CN, straight-chain alkyl groups having 1 to 20 carbon atoms, branched or cyclic alkyl groups having 3 to 20 carbon atoms, aromatic ring systems having 6 to 40 aromatic ring atoms, and heteroaromatic ring systems having 5 to 40 aromatic ring atoms; where said alkyl groups, said aromatic ring systems and said heteroaromatic ring systems are each substituted by R⁷ radicals. Most preferably, R⁶ is the same or different at each instance and is selected from H and D.

Preferably, R¹, R², R³, R⁴ and R⁵ groups, especially R¹, R² and R⁵ groups, are the same or different at each instance and are selected from the following groups:

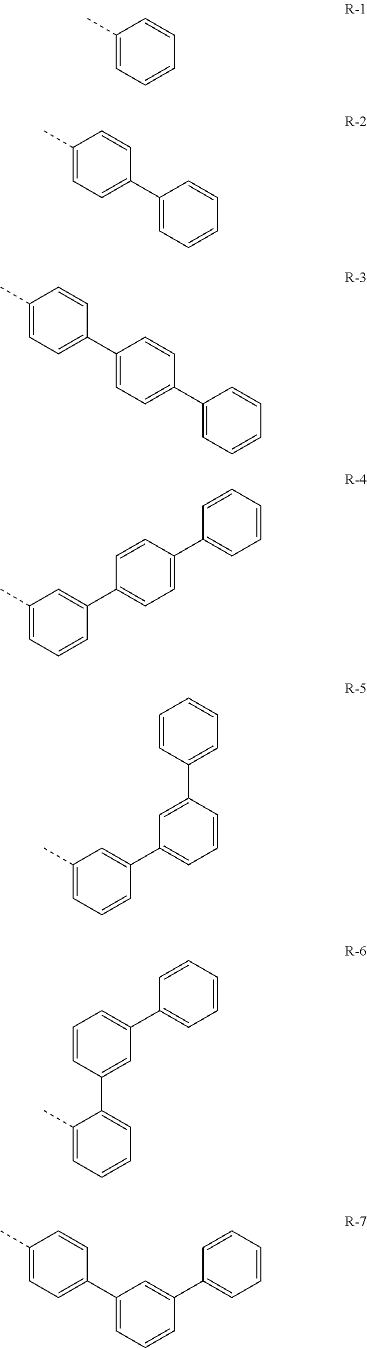

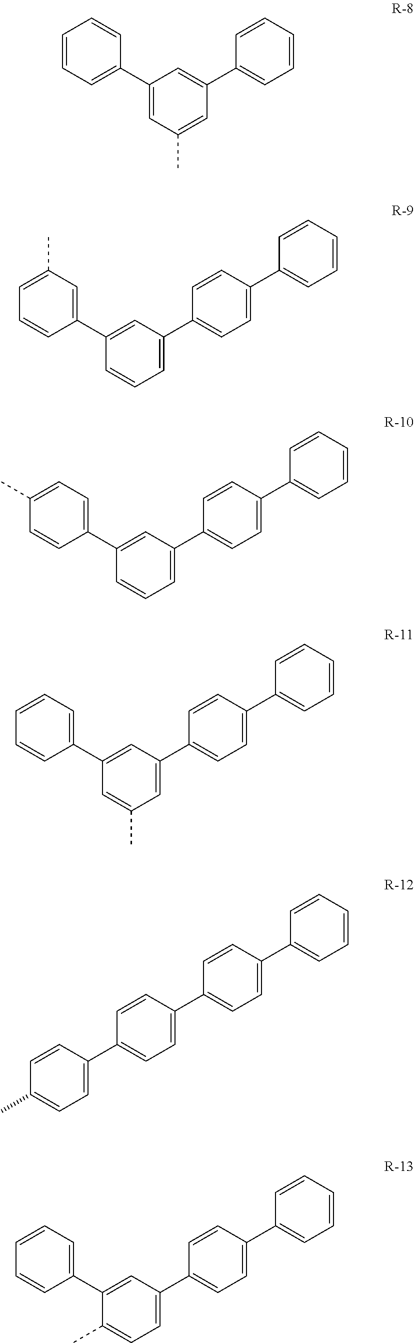

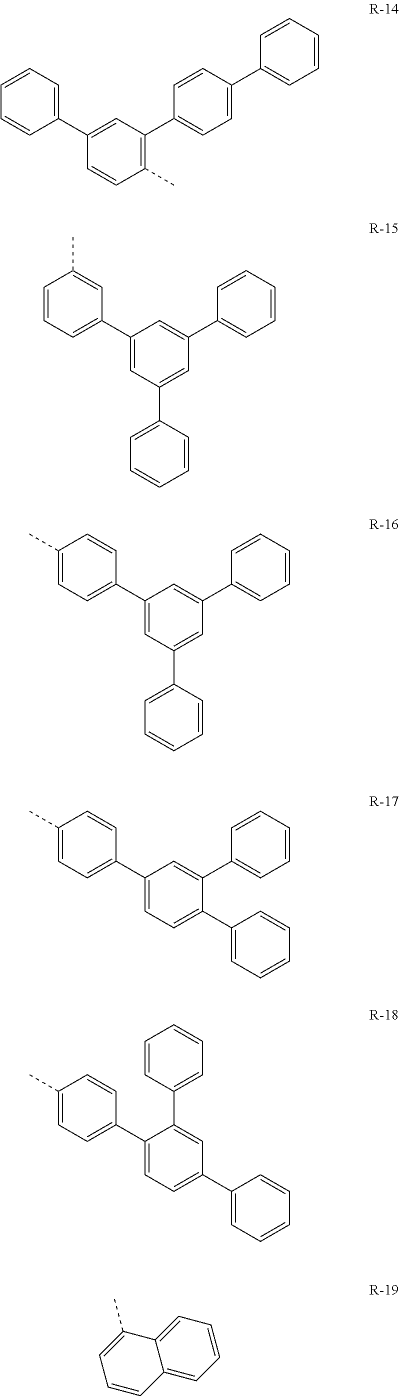

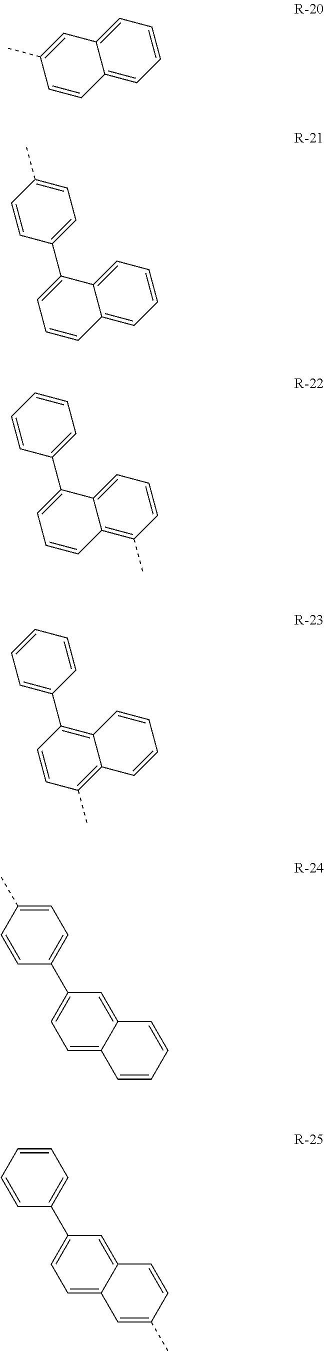

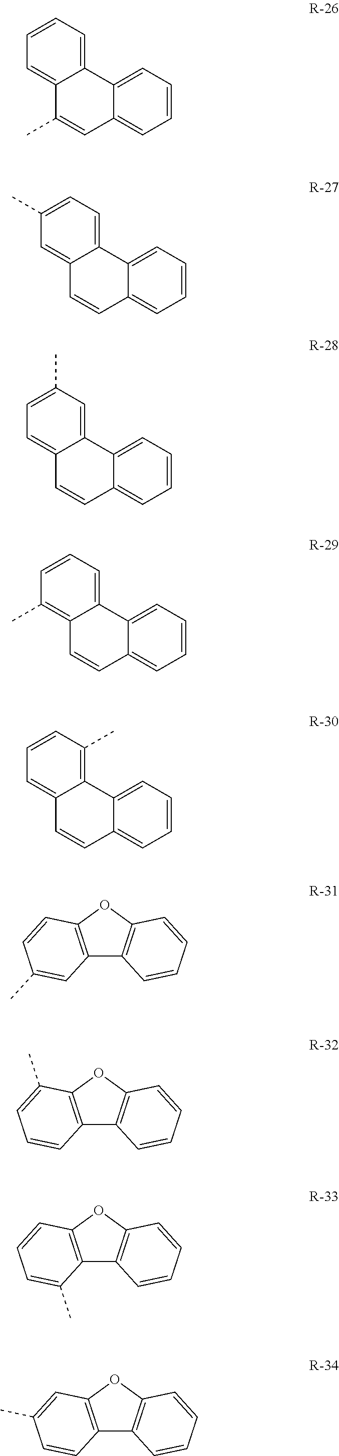

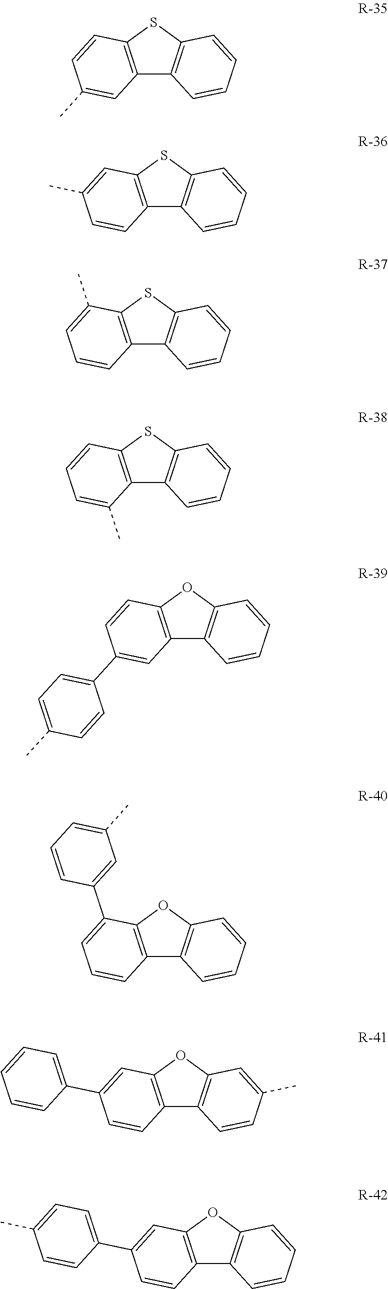

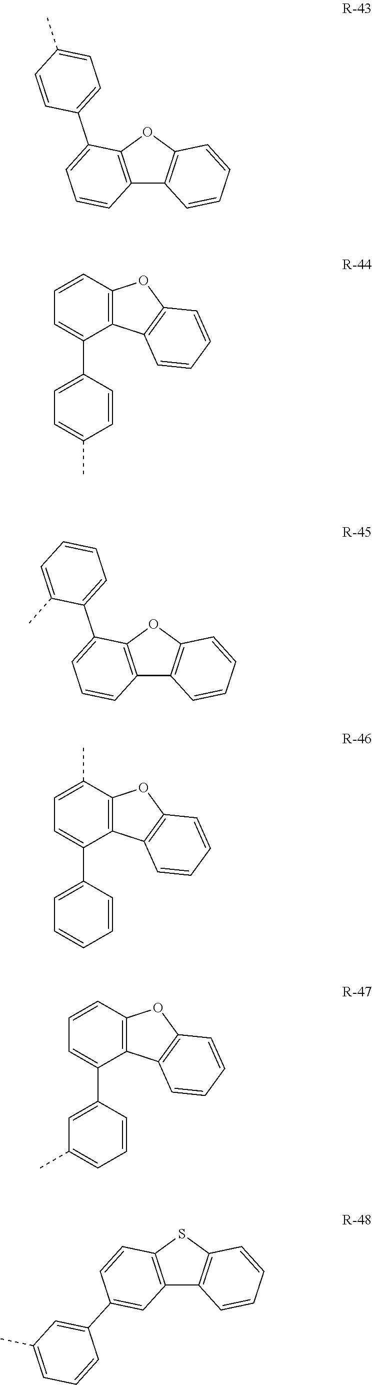

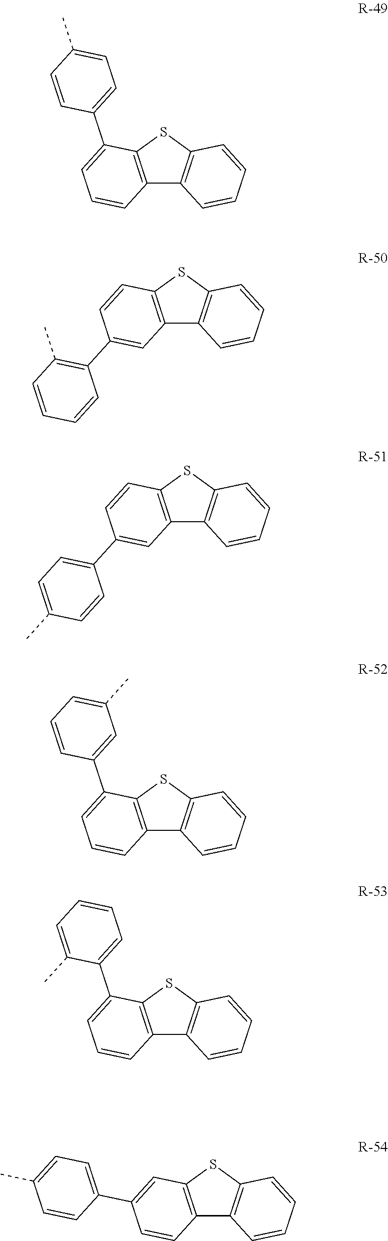

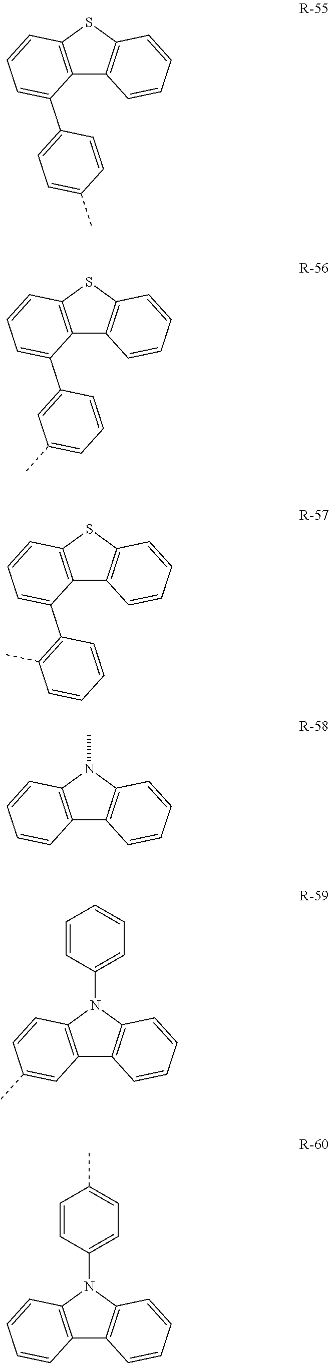

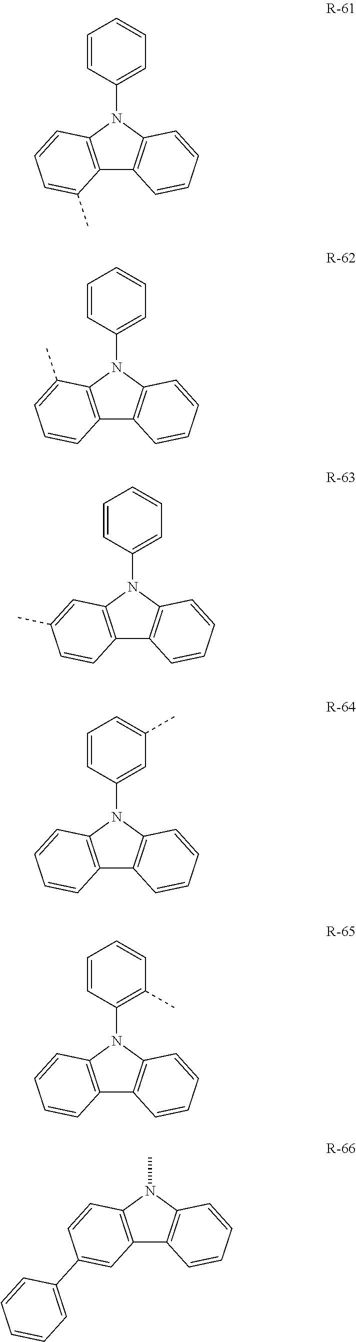

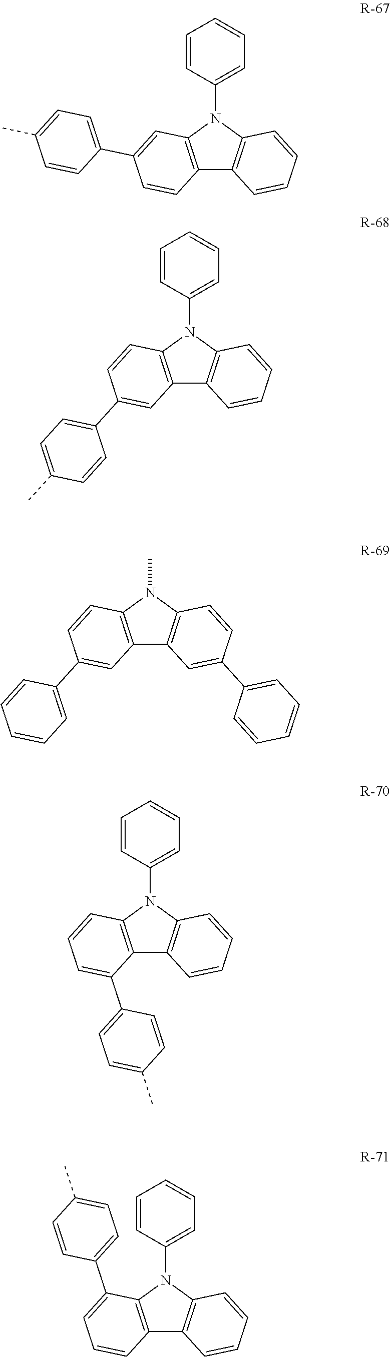

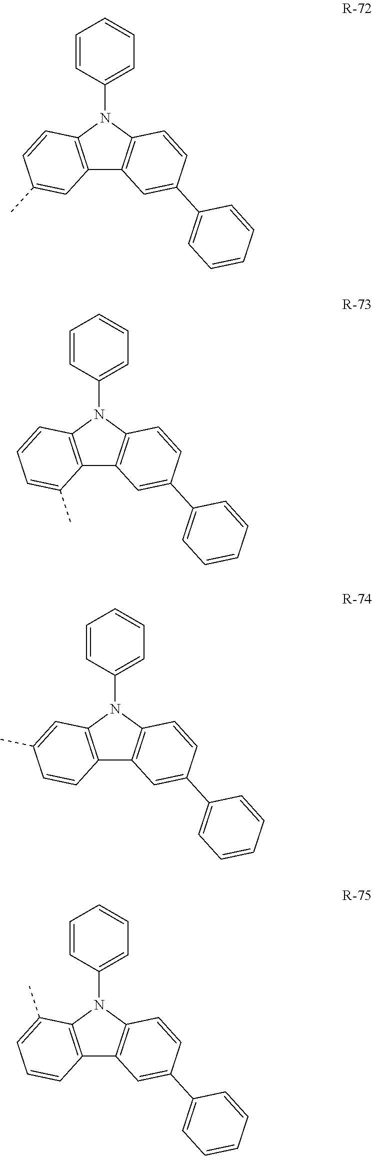

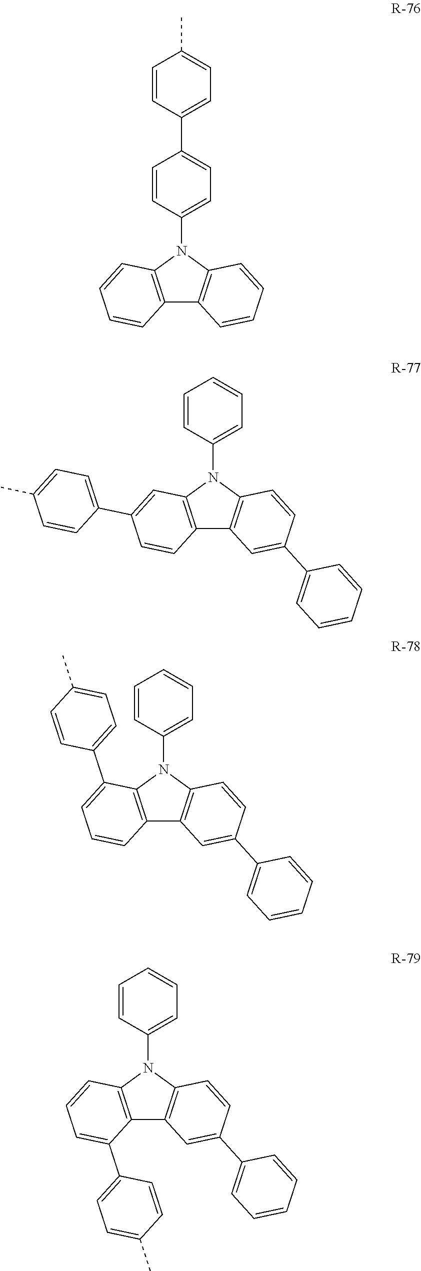

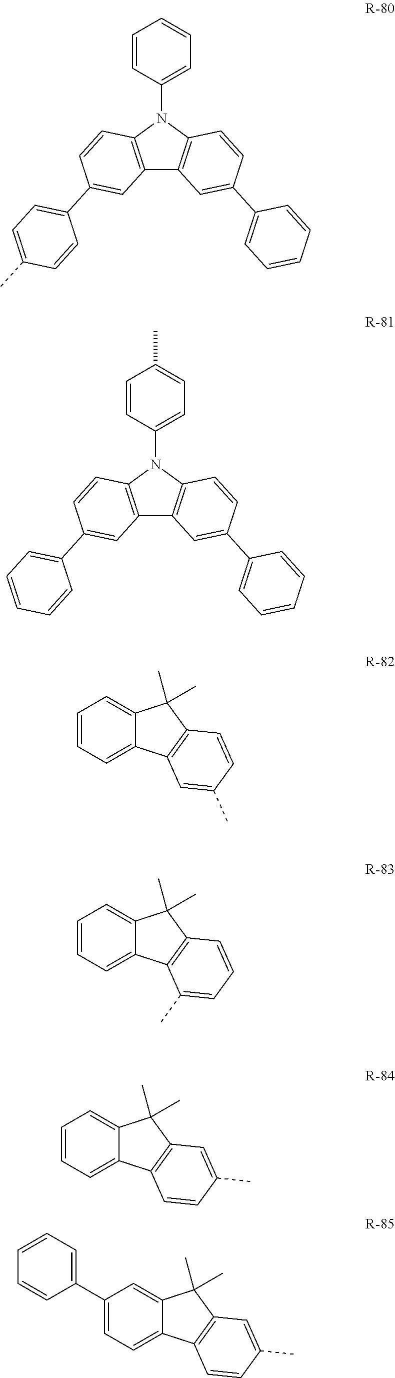

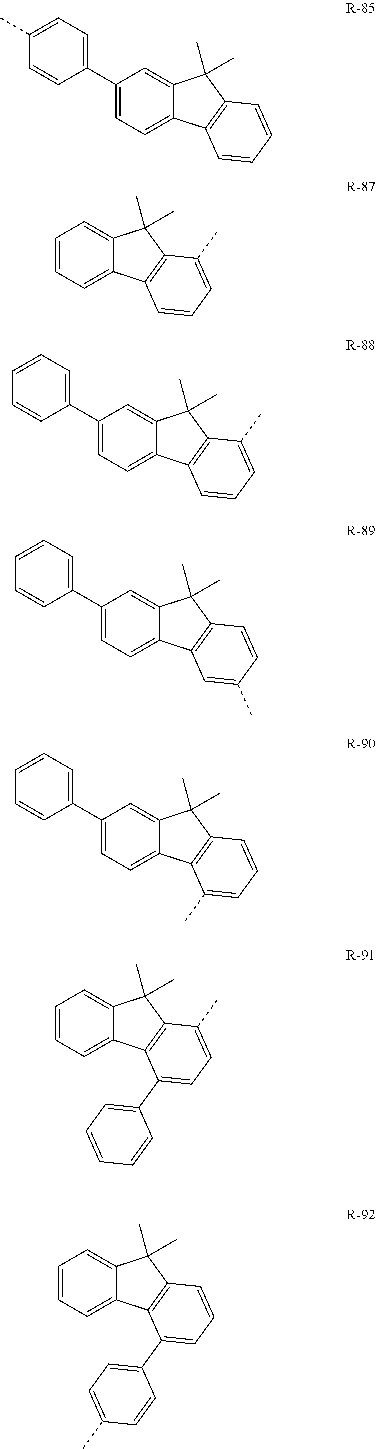

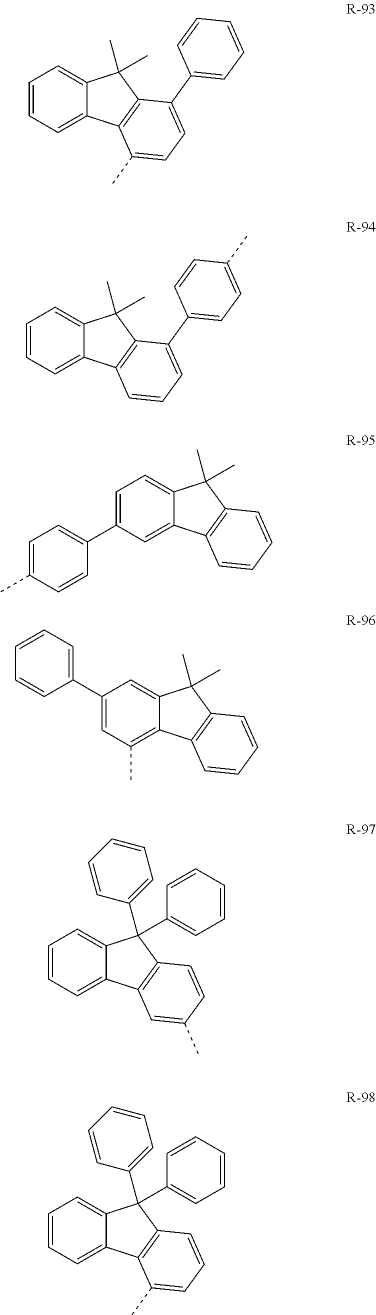

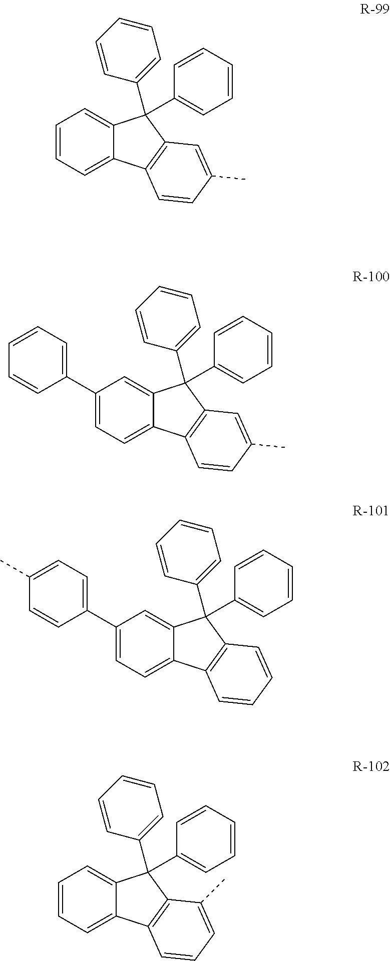

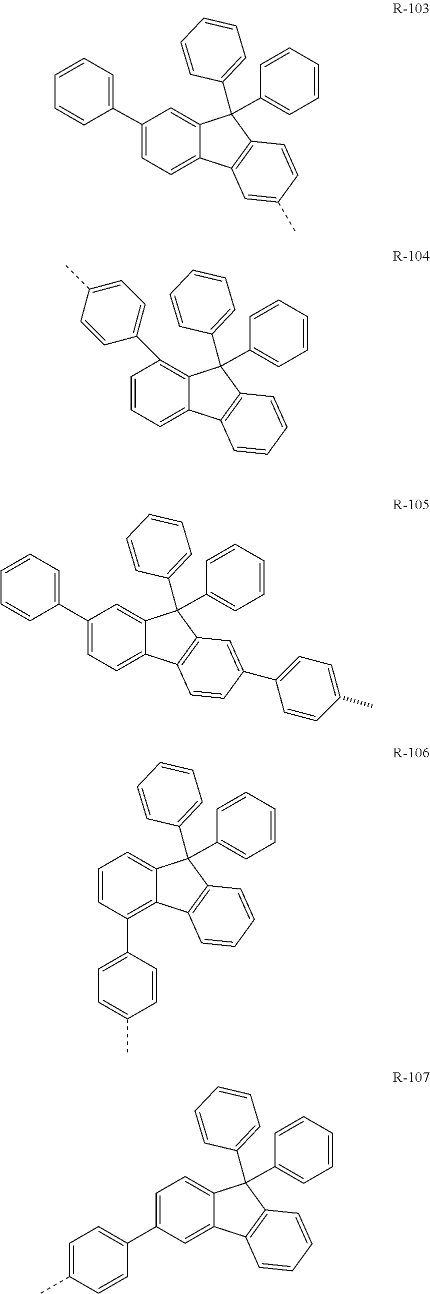

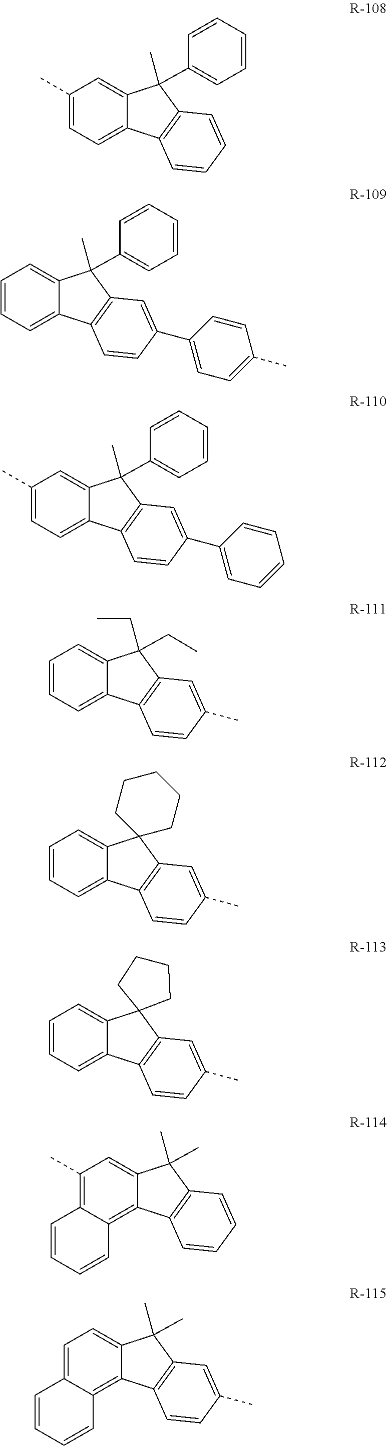

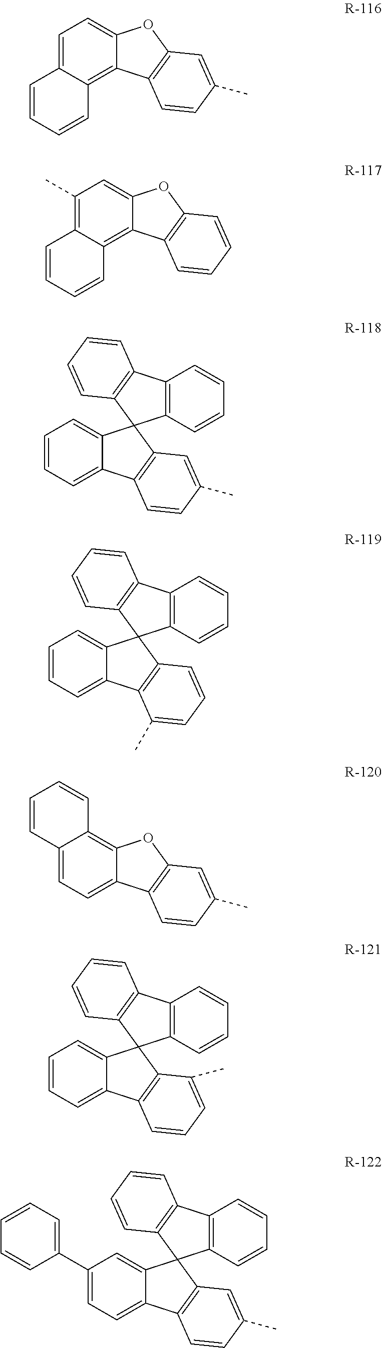

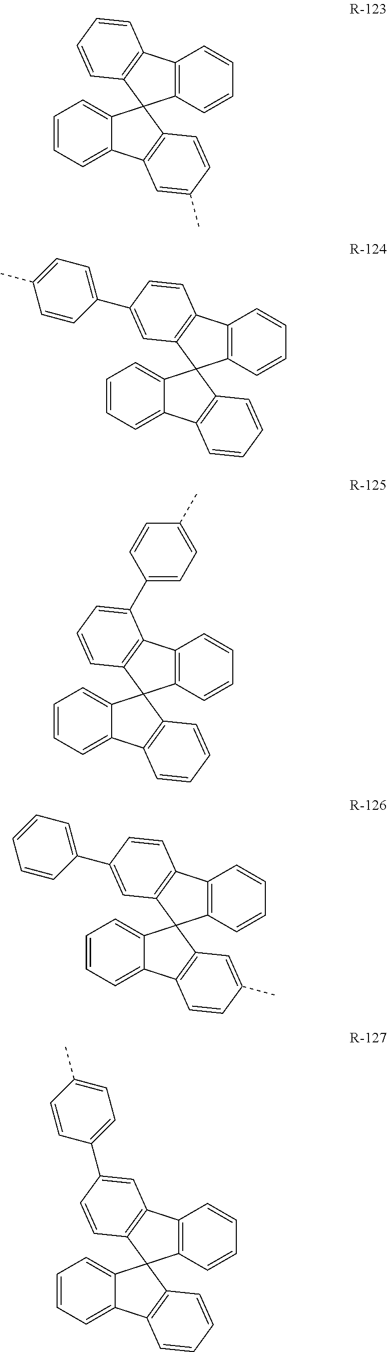

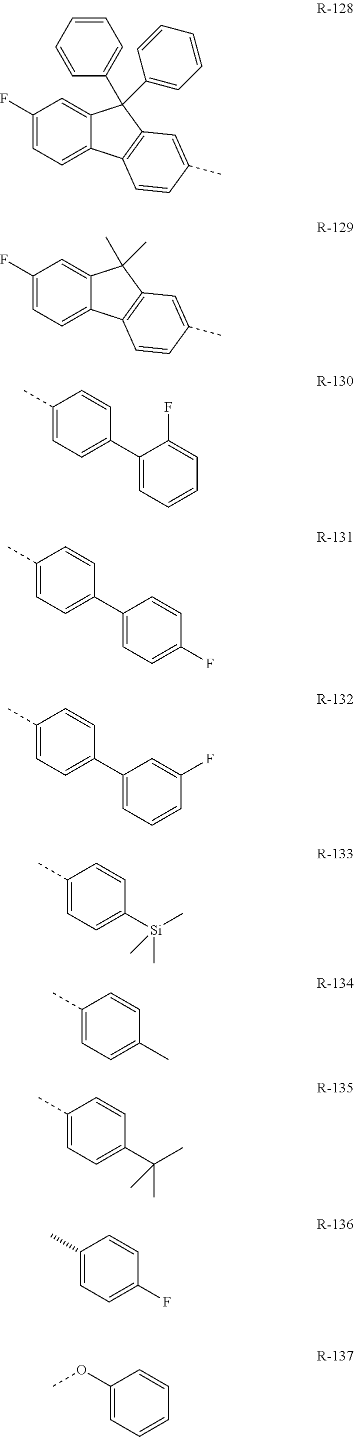

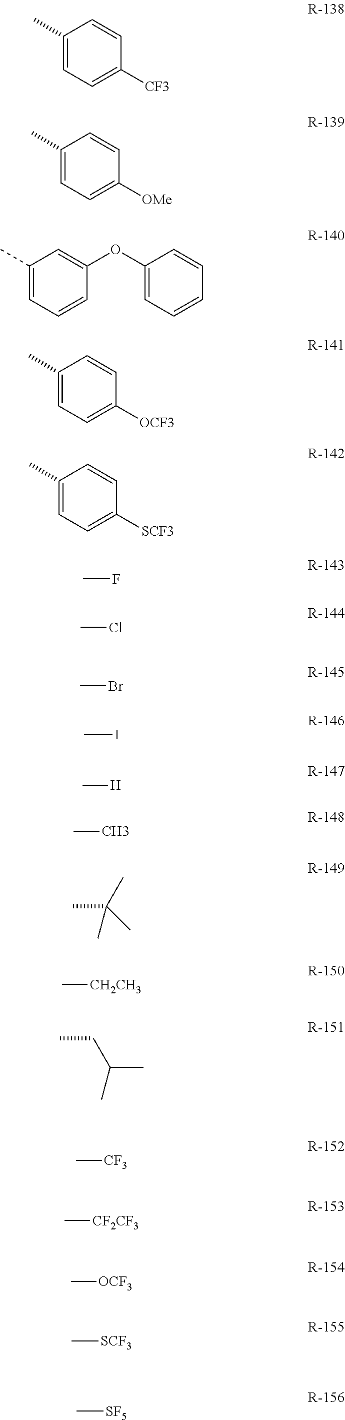

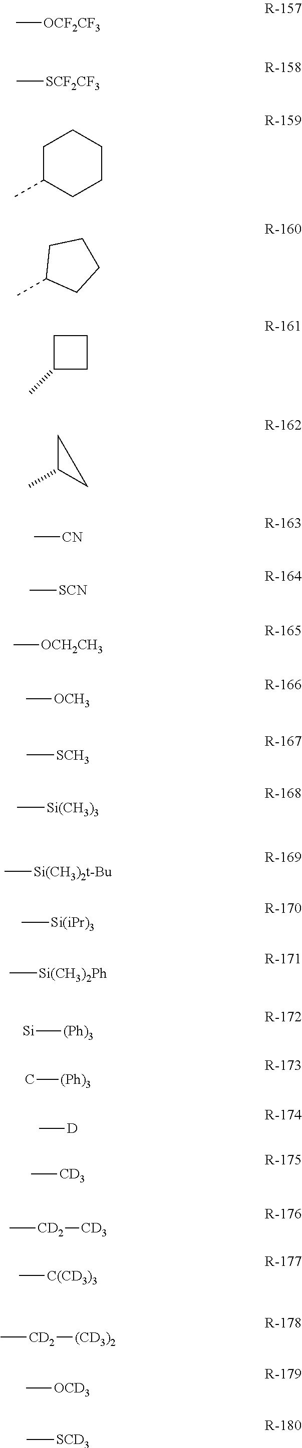

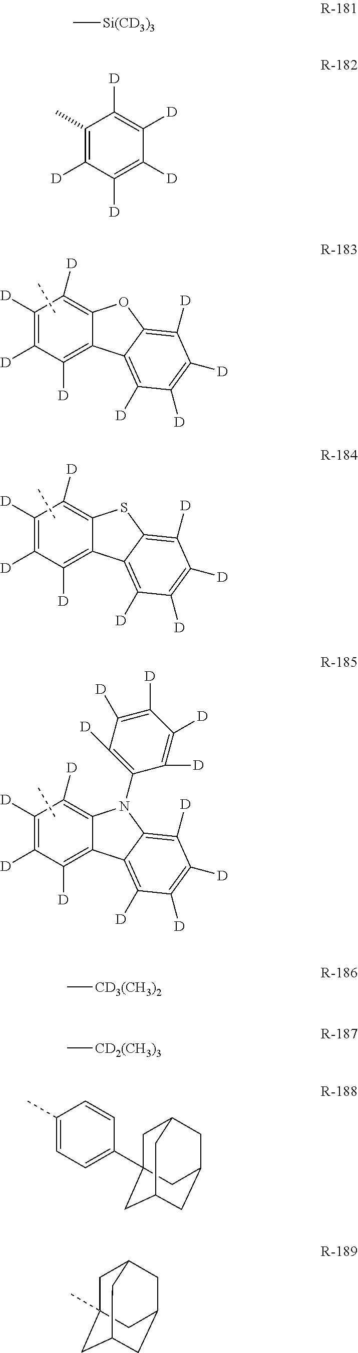

The compound of the formula (I) preferably conforms to one of the following formulae (I-A) to (A-D):

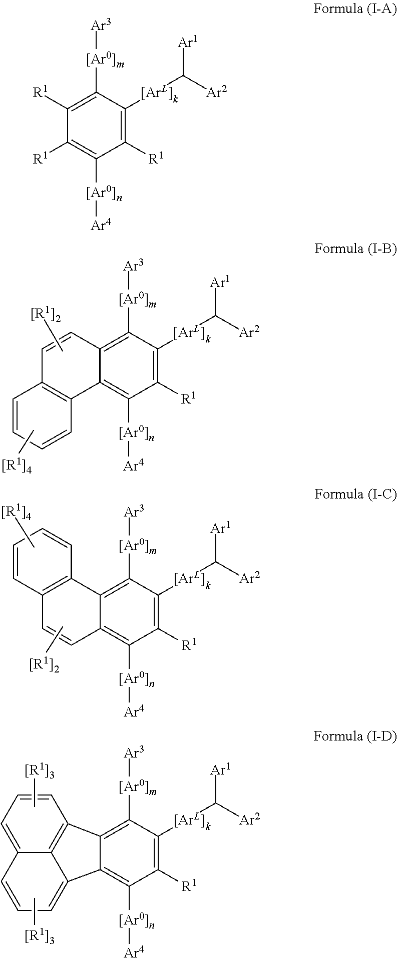

where the variables that occur are as defined for formula (I) and preferably conform to their preferred embodiments. What is meant here by the numerical indices, for example 2 and 4 in the case of the R¹ groups in formula (I-B), is that, in that case, there are 2 to 4 R¹ groups on the ring in question, in accordance with the number of positions shown as unsubstituted on that ring.

Numerical indices that occur in the further embodiments of this application have the same meaning.

Preferred embodiments of the formulae (I-A) to (I-D) are the following formulae:

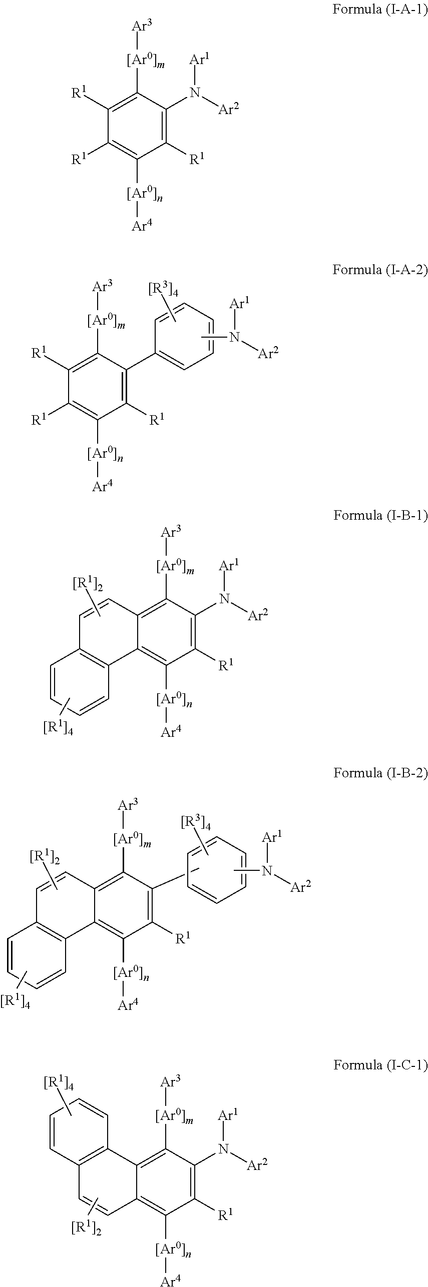

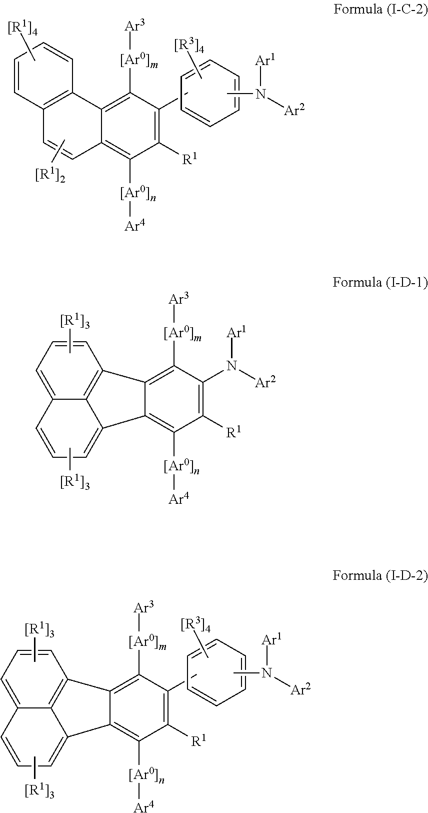

where the variables that occur are as defined for formula (I) and preferably conform to their preferred embodiments.

Preferred embodiments of the formula (I-A) also conform to the following formulae:

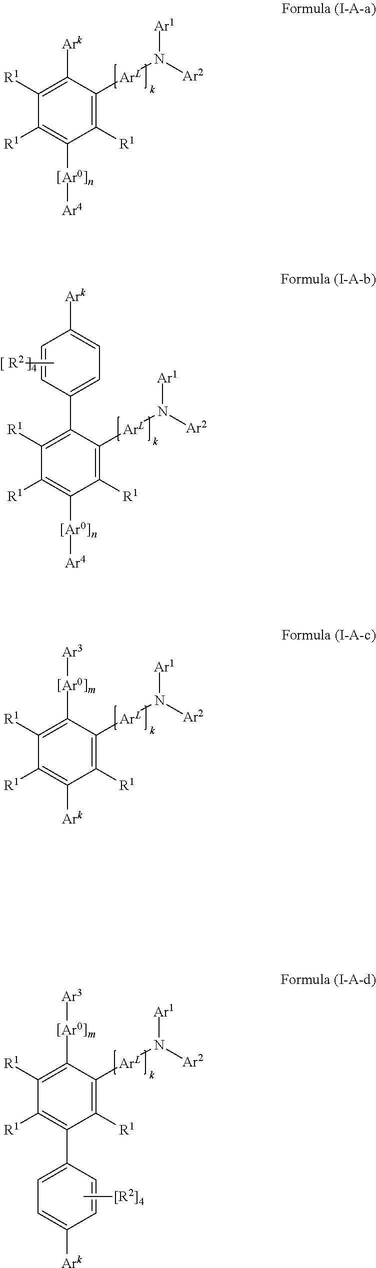

where Ar^{k} is selected from aryl groups which have 10 to 20 aromatic ring atoms and are substituted by R⁵ radicals; and where Ar³ and Ar⁴ are the same or different at each instance and are selected from aromatic ring systems which have 6 to 40 aromatic ring atoms and are substituted by R⁵ radicals, and from heteroaromatic ring systems which have 5 to 40 aromatic ring atoms and are substituted by R⁵ radicals; where the other variables that occur are as defined for formula (I) and preferably conform to their preferred embodiments.

Ar^{k} is preferably selected from naphthyl, phenanthrenyl, anthracenyl, chrysenyl, pyrenyl and fluoranthenyl, each substituted by R⁵ radicals.

Especially preferably, Ar^{k} is selected from the groups of the formulae (Ar^{k}-1) to (Ar^{k}-6), as defined above, where the variables that occur are as defined above and preferably conform to their preferred embodiments, and where the dotted line is the bond of the Ar^{k} group to the rest of the compound.

Among the abovementioned formulae (I-A-a) to (I-A-d), particular preference is given to the formulae (I-A-a), (I-A-b) and (I-A-d), very particular preference to the formula (I-A-a).

Particular preference is given to the following formulae:

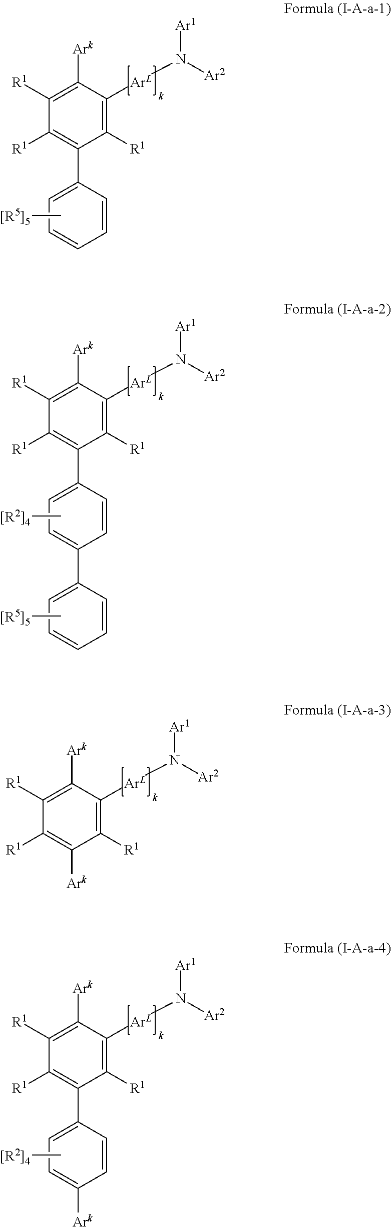

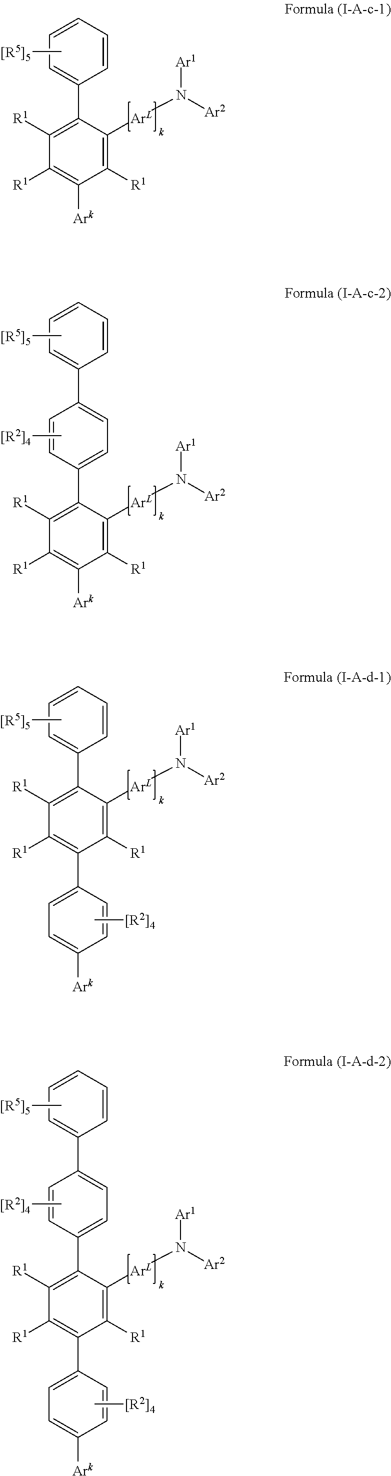

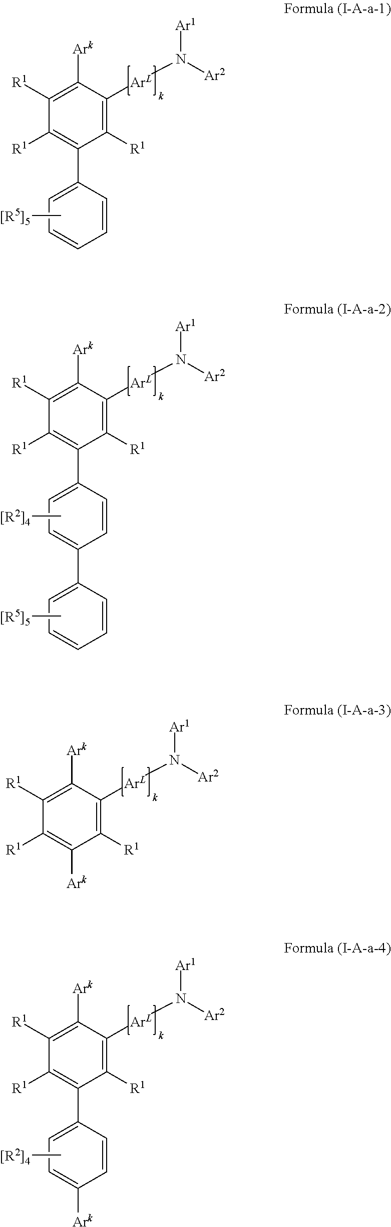

where Ar^{k} is selected from aryl groups which have 10 to 20 aromatic ring atoms and are substituted by R⁵ radicals, preferably selected from the formulae (Ar^{k}-1) to (Ar^{k}-6) as defined above, and where the other variables that occur are as defined for formula (I) and preferably conform to their preferred embodiments. In a preferred embodiment, k in one of the abovementioned formulae is 0. In an alternative preferred embodiment, k=1 and Ar^{L} is a phenyl group substituted by R³ radicals.

Among the abovementioned formulae, preference is given to the formulae (I-A-a-1), (I-A-a-2), (I-A-a-4), (I-A-b-1) to (I-A-b-4), (I-A-c-1), (I-A-c-2), (I-A-d-1) and (I-A-d-2).

Among the abovementioned formulae, preference is likewise given to the formulae (I-A-a-1), (I-A-a-2), (I-A-a-4), (I-A-b-1), (I-A-b-2), (I-A-b-4), (I-A-d-1) and (I-A-d-2).

Further preferred embodiments of compounds of the formula (I) conform to the following formulae:

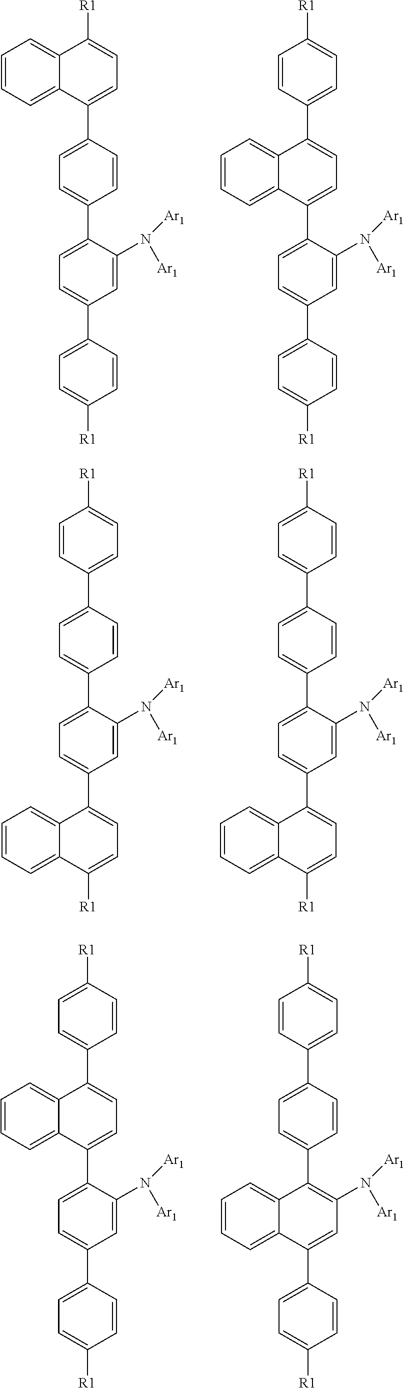

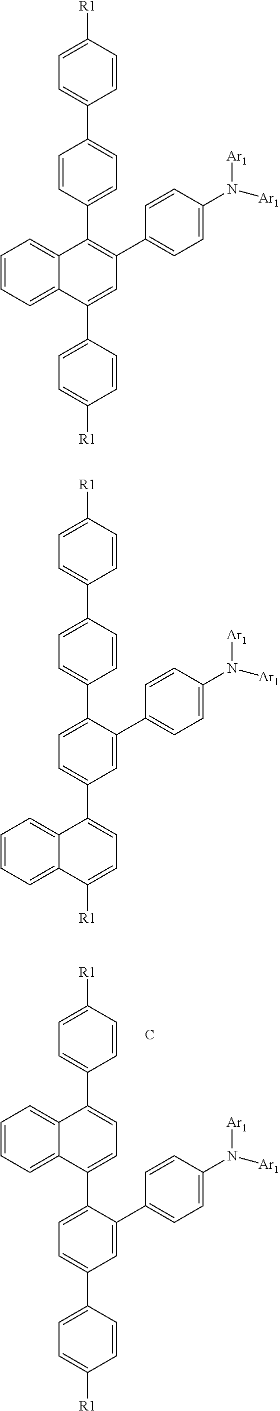

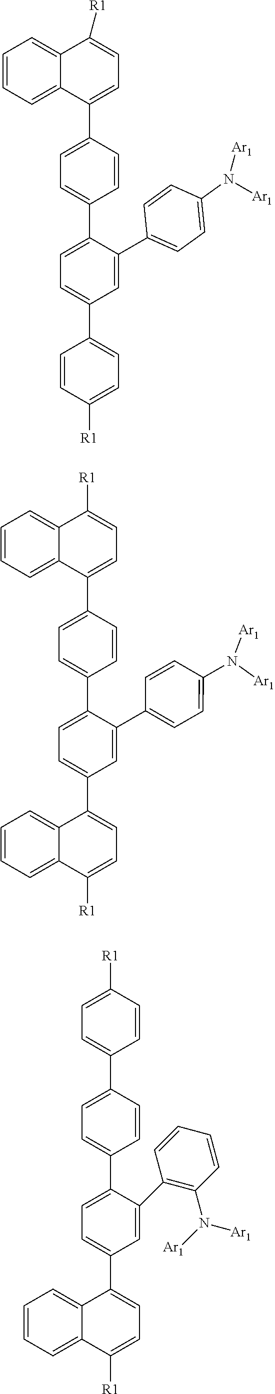

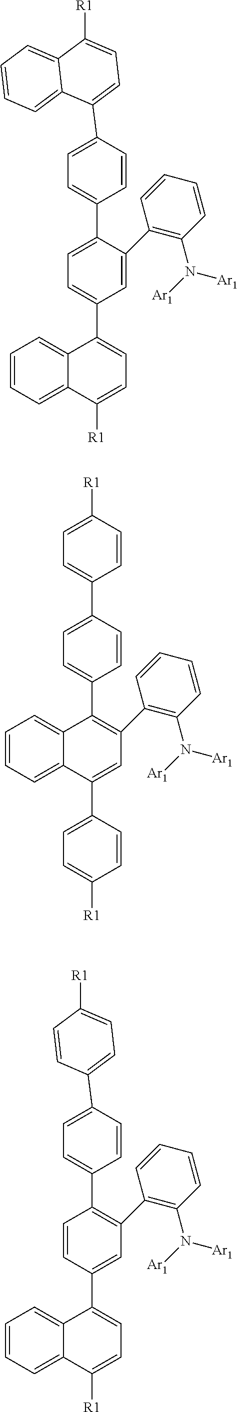

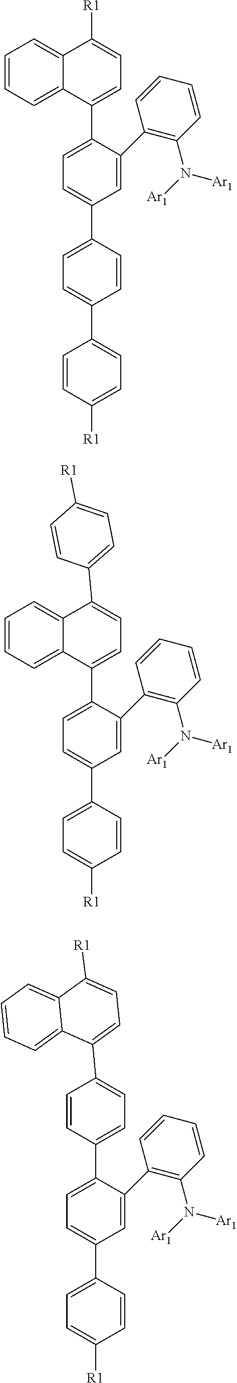

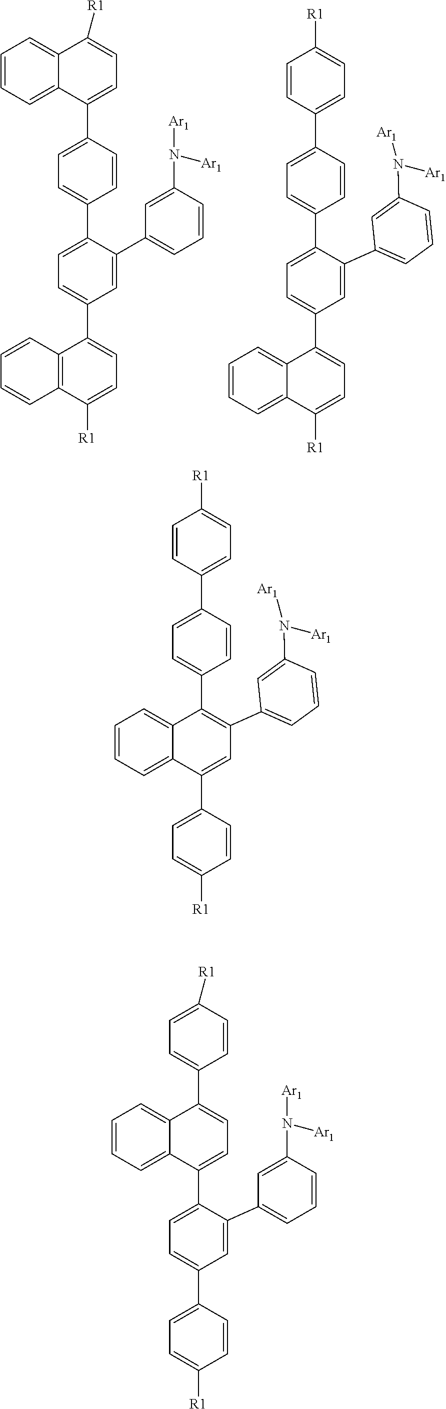

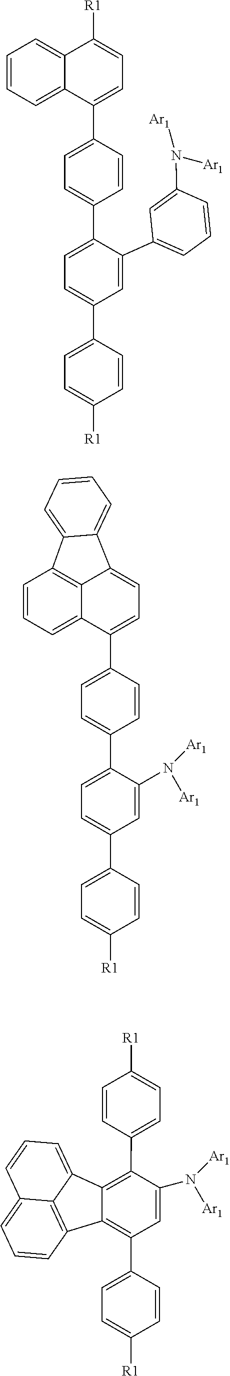

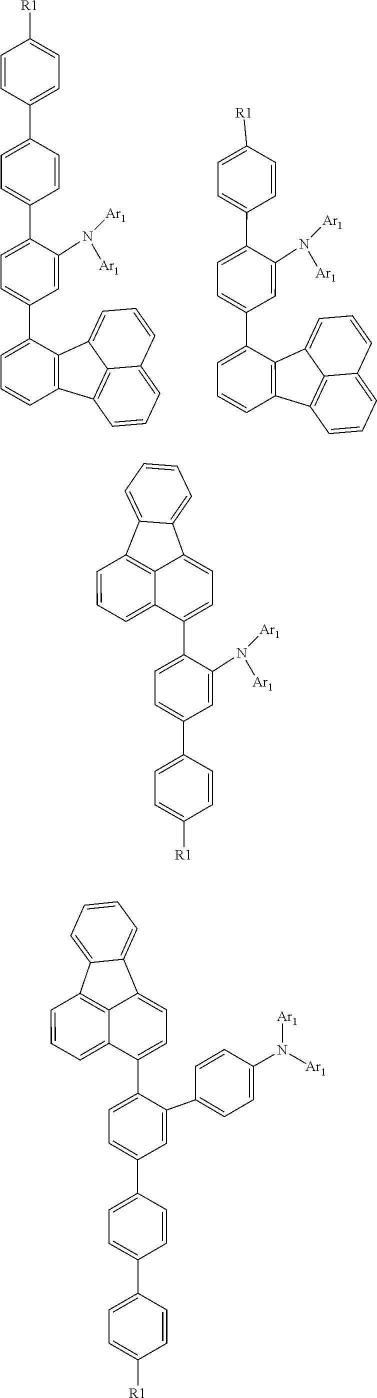

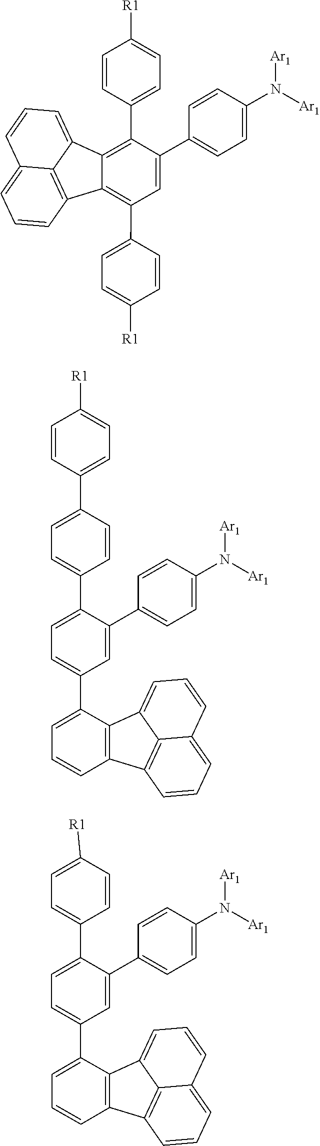

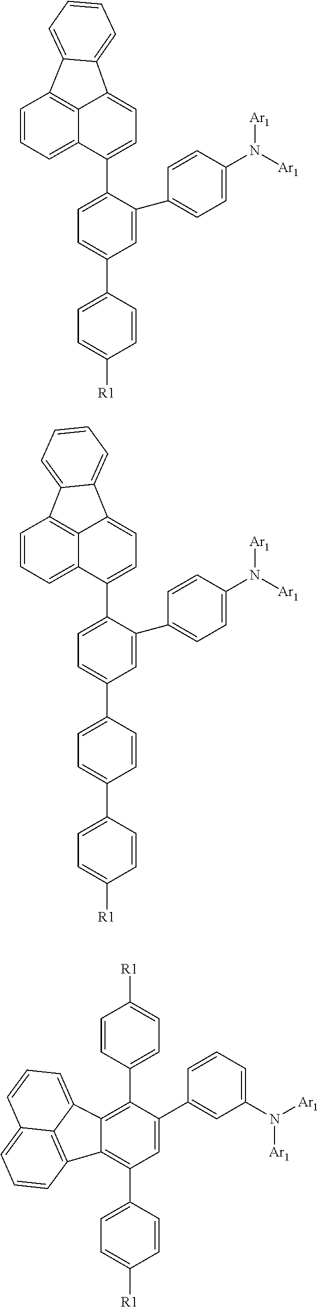

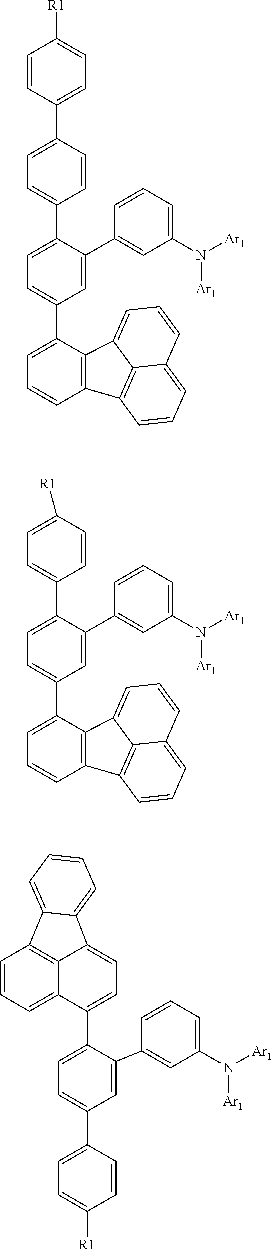

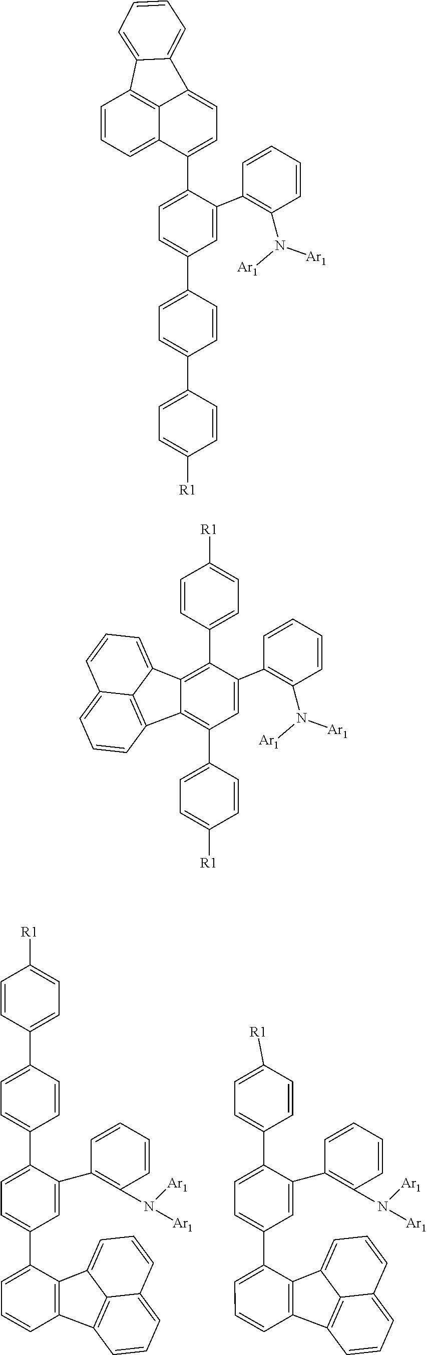

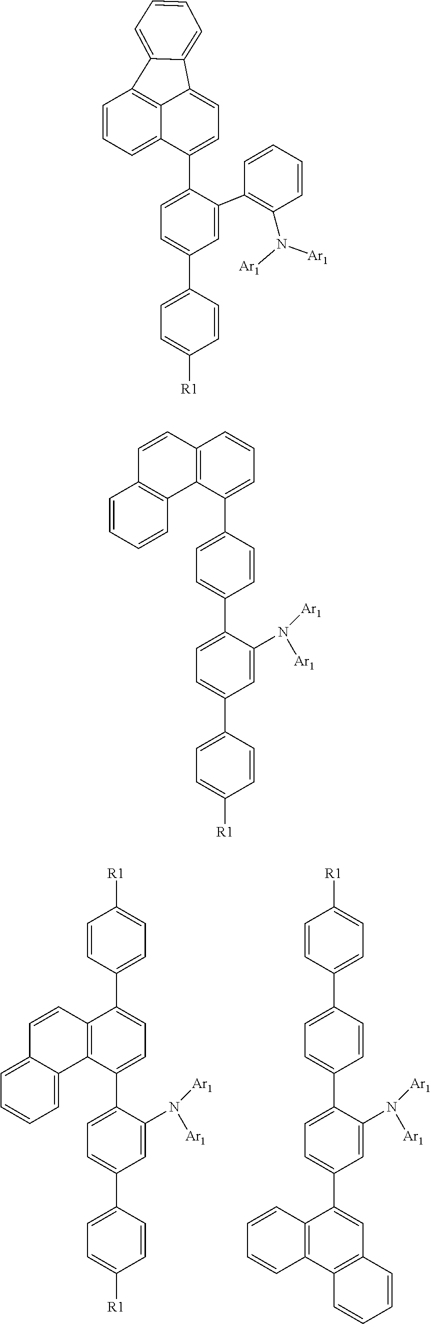

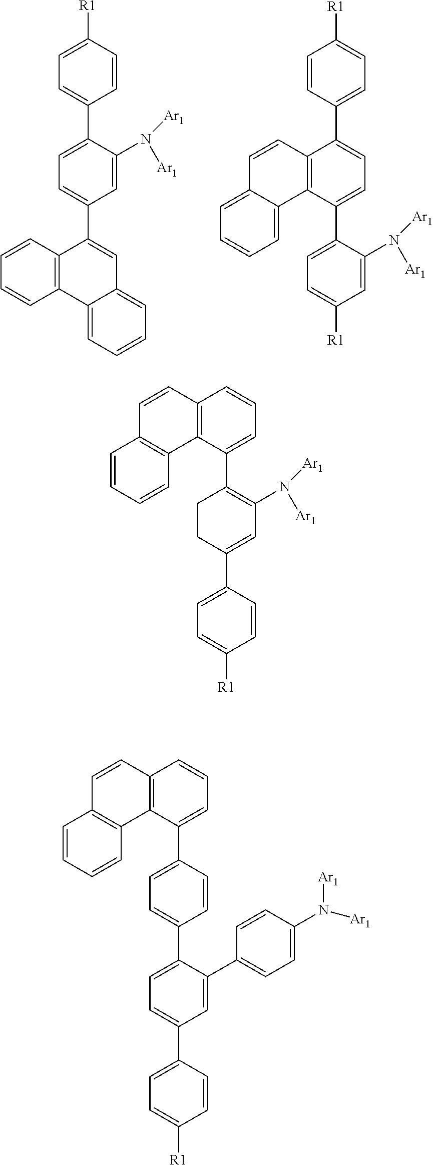

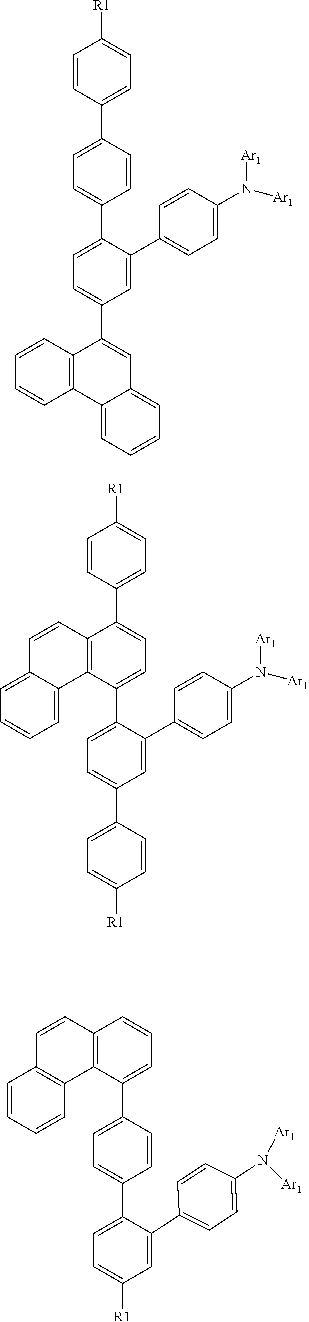

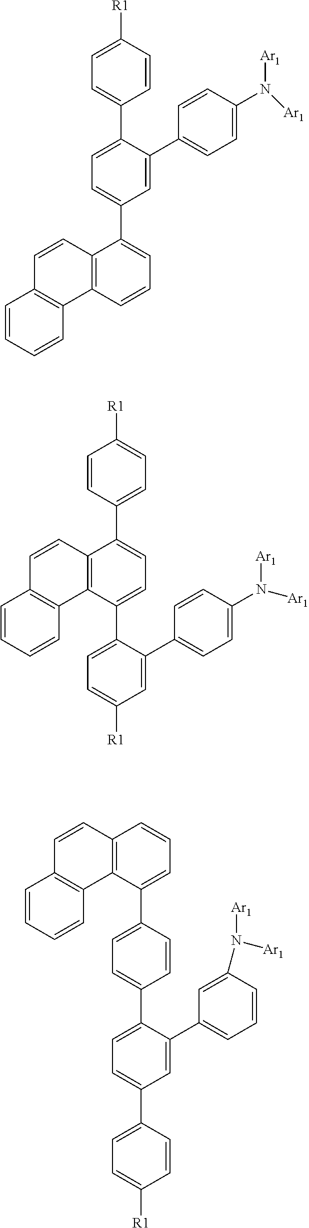

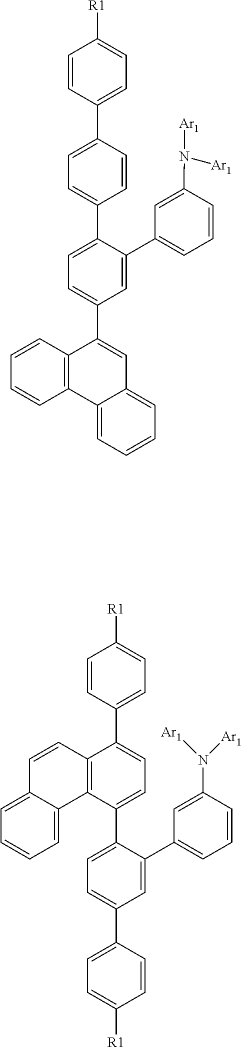

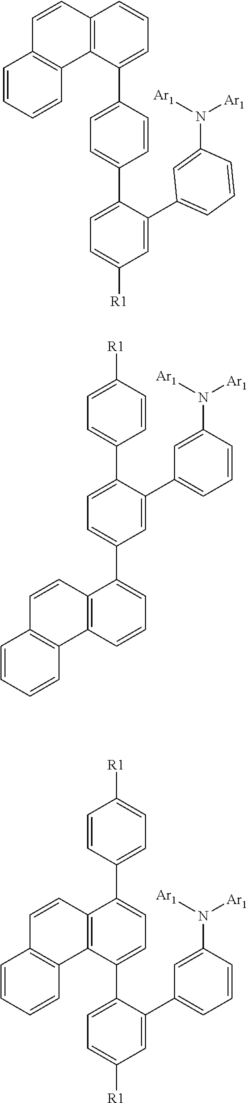

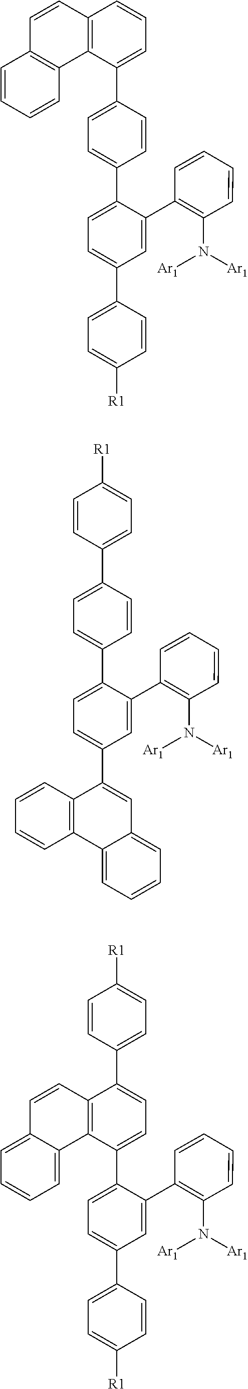

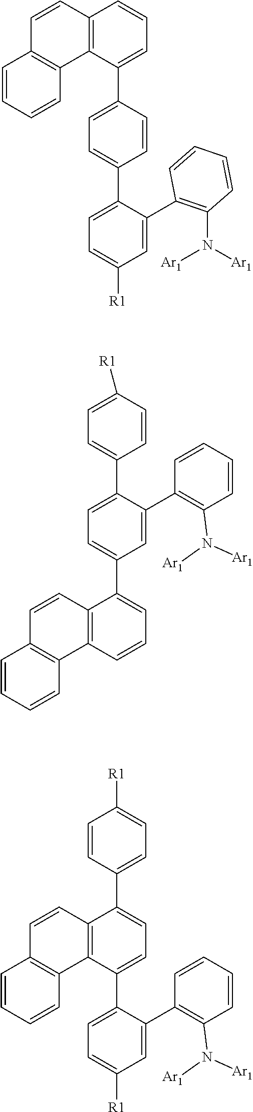

where the “Ar¹” groups in the above formulae each correspond to Ar¹ and Ar² as defined above, and where “R¹” corresponds to R⁵ as defined above.

In a preferred embodiment, the following embodiments of the variables are applicable in combination to the compounds of the formula (I):
Z¹ is CR¹, and

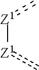

units cannot be replaced by units of the formulae (Z-1) to (Z-6);
Ar¹ and Ar² are the same or different at each instance and are selected from benzene, biphenyl, terphenyl, quaterphenyl, naphthyl, fluorenyl, especially 9,9′-dimethylfluorenyl and 9,9′-diphenylfluorenyl, benzofluorenyl, spirobifluorenyl, indenofluorenyl, indenocarbazolyl, dibenzofuranyl, dibenzothiophenyl, carbazolyl, benzofuranyl, benzothiophenyl, benzofused dibenzofuranyl, benzofused dibenzothiophenyl, and phenyl substituted by a group selected from naphthyl, fluorenyl, spirobifluorenyl, dibenzofuranyl, dibenzothiophenyl, carbazolyl, pyridyl, pyrimidyl and triazinyl, where the groups are each substituted by R⁴ radicals;
Ar³ and Ar⁴ groups are the same or different at each instance and are selected from phenyl, biphenyl, terphenyl, quaterphenyl, naphthyl, phenanthrenyl, fluoranthenyl, fluorenyl, especially 9,9′-dimethylfluorenyl and 9,9′-diphenylfluorenyl, benzofluorenyl, spirobifluorenyl, indenofluorenyl, indenocarbazolyl, dibenzofuranyl, dibenzothiophenyl, carbazolyl, benzofuranyl, benzothiophenyl, benzofused dibenzofuranyl, benzofused dibenzothiophenyl, and phenyl substituted by a group selected from naphthyl, fluorenyl, spirobifluorenyl, dibenzofuranyl, dibenzothiophenyl, carbazolyl, pyridyl, pyrimidyl and triazinyl, where the groups are each substituted by R⁵ radicals; where at least one group selected from the groups Ar³ and Ar⁴ must be selected from naphthyl, anthracenyl, phenanthrenyl, fluoranthenyl and triphenylenyl, each substituted by R⁵ radicals;
Ar^{L} is selected from phenyl, biphenyl, naphthyl and fluorenyl, each substituted by R³ radicals;
R¹ is the same or different and is selected from H, D, F, CN, straight-chain alkyl groups having 1 to 20 carbon atoms, branched or cyclic alkyl groups having 3 to 20 carbon atoms; where said alkyl groups are each substituted by R⁶ radicals;
R², R³ and R⁵ are the same or different and are selected from H, D, F, CN, straight-chain alkyl groups having 1 to 20 carbon atoms, branched or cyclic alkyl groups having 3 to 20 carbon atoms, aromatic ring systems having 6 to 40 aromatic ring atoms; where said alkyl groups and said aromatic ring systems are each substituted by R⁶ radicals;
R⁴ is the same or different at each instance and is selected from H, D, F, CN, straight-chain alkyl groups having 1 to 20 carbon atoms, branched or cyclic alkyl groups having 3 to 20 carbon atoms, aromatic ring systems having 6 to 40 aromatic ring atoms, and heteroaromatic ring systems having 5 to 40 aromatic ring atoms; where said alkyl groups, said aromatic ring systems and said heteroaromatic ring systems are each substituted by R⁶ radicals;
R⁶ is the same or different at each instance and is selected from H and D;
m is 0 or 1;
n is 0 or 1.

In general, it is preferable that preferred embodiments disclosed in the present application are combined with one another, even though this is not explicitly detailed as such in the abovementioned combination of preferred embodiments.

Preferred embodiments of compounds of formula (I) are listed below:

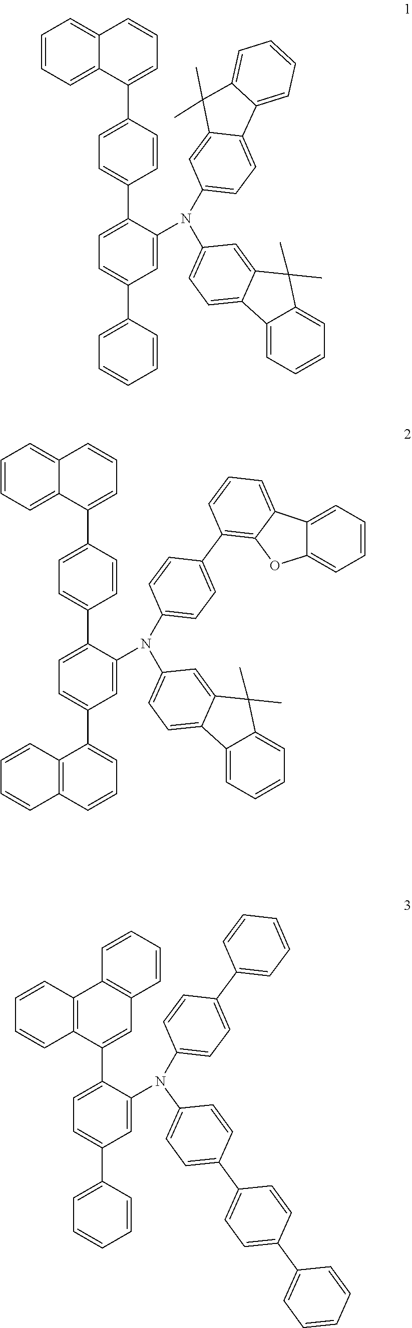 [SMILES: CC1(C)c2ccccc2-c2ccc(N(c3ccc4c(c3)C(C)(C)c3ccccc3-4)c3cc(-c4ccccc4)ccc3-c3ccc(-c4cccc5ccccc45)cc3)cc21 ; CC1(C)c2ccccc2-c2ccc(N(c3ccc(-c4cccc5c4oc4ccccc45)cc3)c3cc(-c4cccc5ccccc45)ccc3-c3ccc(-c4cccc5ccccc45)cc3)cc21 ; c1ccc(-c2ccc(-c3ccc(N(c4ccc(-c5ccccc5)cc4)c4cc(-c5ccccc5)ccc4-c4cc5ccccc5c5ccccc45)cc3)cc2)cc1]

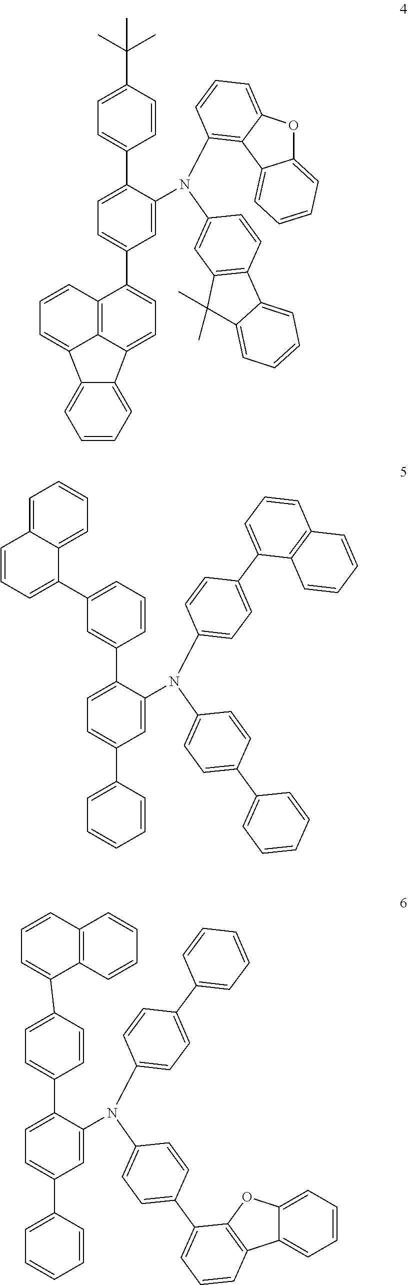 [SMILES: CC(C)(C)c1ccc(-c2ccc(-c3ccc4c5c(cccc35)-c3ccccc3-4)cc2N(c2ccc3c(c2)C(C)(C)c2ccccc2-3)c2cccc3oc4ccccc4c23)cc1 ; c1ccc(-c2ccc(N(c3ccc(-c4cccc5ccccc45)cc3)c3cc(-c4ccccc4)ccc3-c3cccc(-c4cccc5ccccc45)c3)cc2)cc1 ; c1ccc(-c2ccc(N(c3ccc(-c4cccc5c4oc4ccccc45)cc3)c3cc(-c4ccccc4)ccc3-c3ccc(-c4cccc5ccccc45)cc3)cc2)cc1]

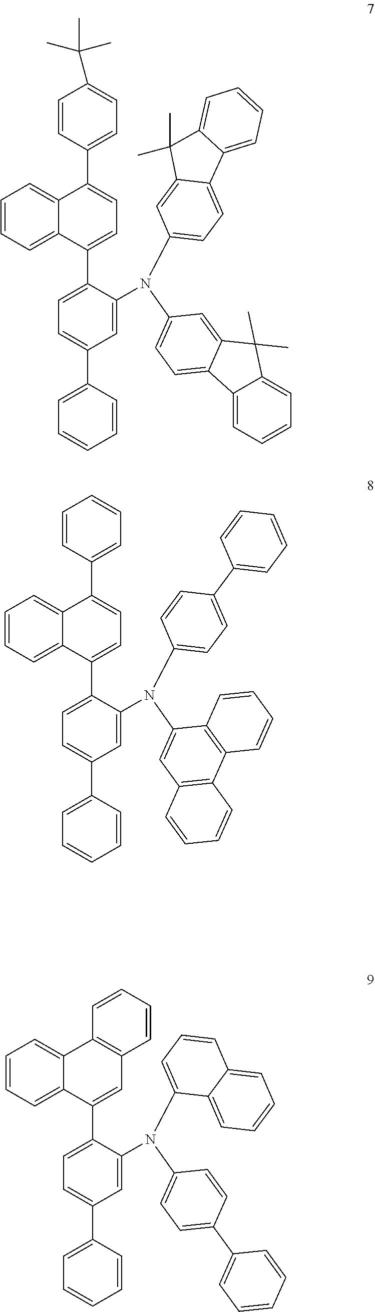

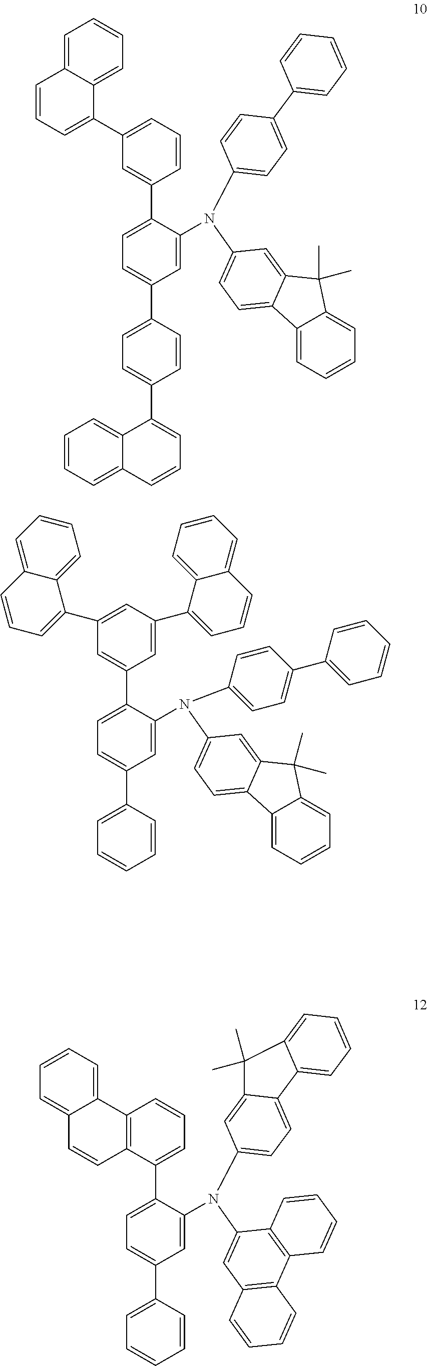

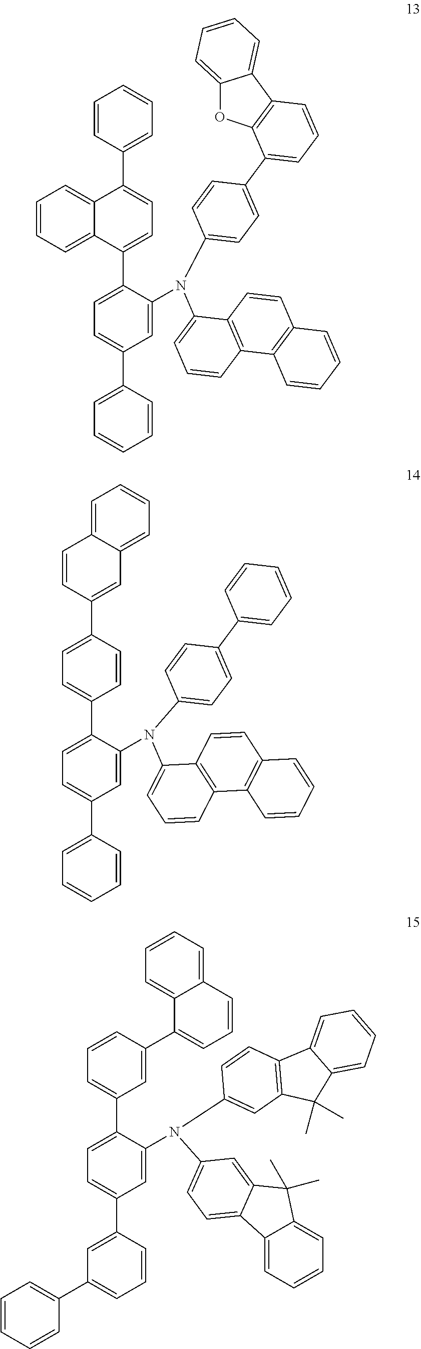

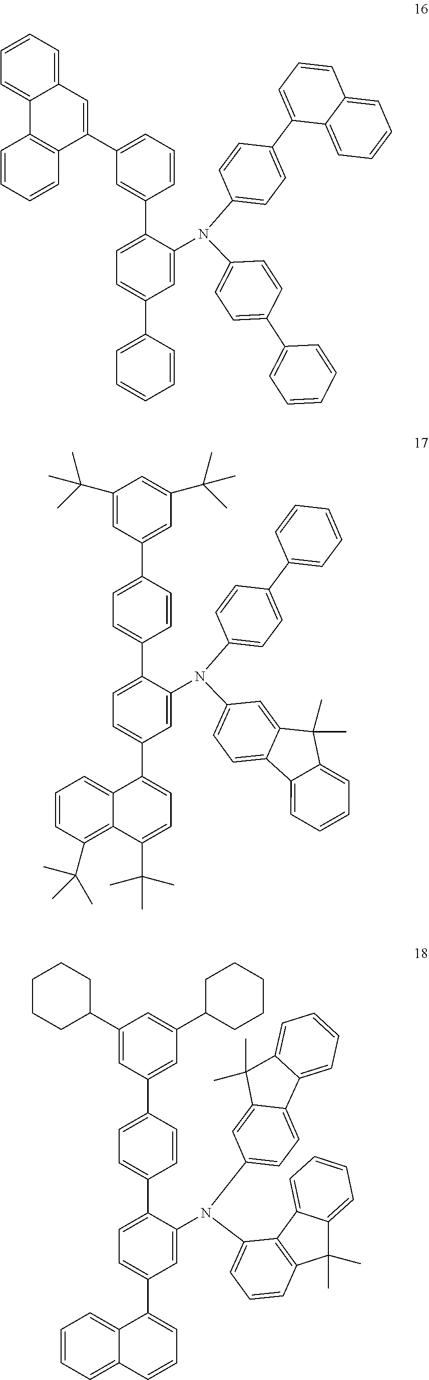 [SMILES: c1ccc(-c2ccc(N(c3ccc(-c4cccc5ccccc45)cc3)c3cc(-c4ccccc4)ccc3-c3cccc(-c4cc5ccccc5c5ccccc45)c3)cc2)cc1 ; CC(C)(C)c1cc(-c2ccc(-c3ccc(-c4ccc(C(C)(C)C)c5c(C(C)(C)C)cccc45)cc3N(c3ccc(-c4ccccc4)cc3)c3ccc4c(c3)C(C)(C)c3ccccc3-4)cc2)cc(C(C)(C)C)c1 ; CC1(C)c2ccccc2-c2ccc(N(c3cc(-c4cccc5ccccc45)ccc3-c3ccc(-c4cc(C5CCCCC5)cc(C5CCCCC5)c4)cc3)c3cccc4c3-c3ccccc3C4(C)C)cc21]

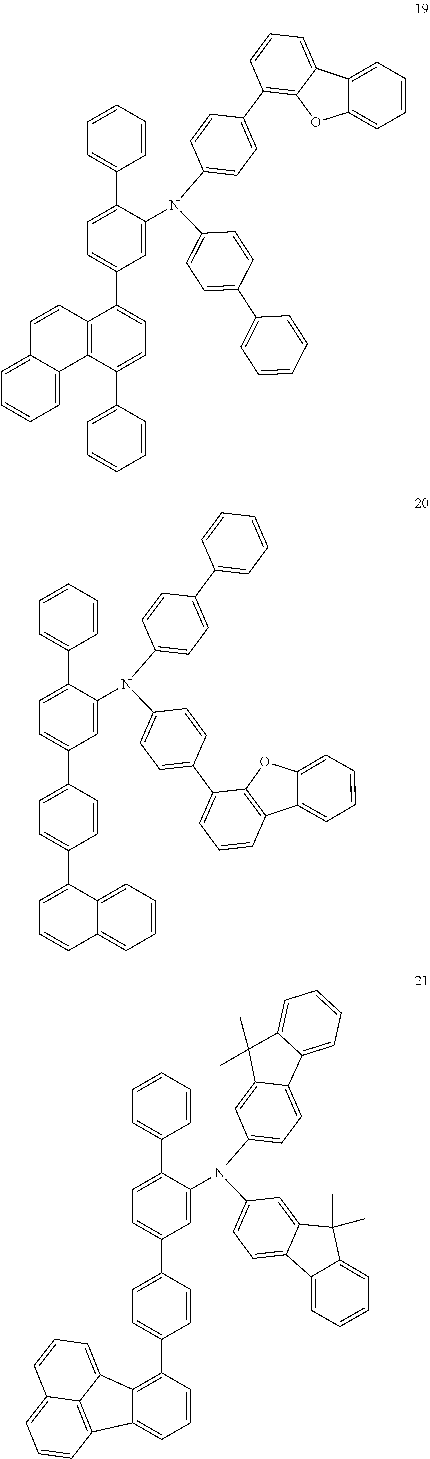 [SMILES: c1ccc(-c2ccc(N(c3ccc(-c4cccc5c4oc4ccccc45)cc3)c3cc(-c4ccc(-c5ccccc5)c5c4ccc4ccccc45)ccc3-c3ccccc3)cc2)cc1 ; c1ccc(-c2ccc(N(c3ccc(-c4cccc5c4oc4ccccc45)cc3)c3cc(-c4ccc(-c5cccc6ccccc56)cc4)ccc3-c3ccccc3)cc2)cc1 ; CC1(C)c2ccccc2-c2ccc(N(c3ccc4c(c3)C(C)(C)c3ccccc3-4)c3cc(-c4ccc(-c5cccc6c5-c5cccc7cccc-6c57)cc4)ccc3-c3ccccc3)cc21]

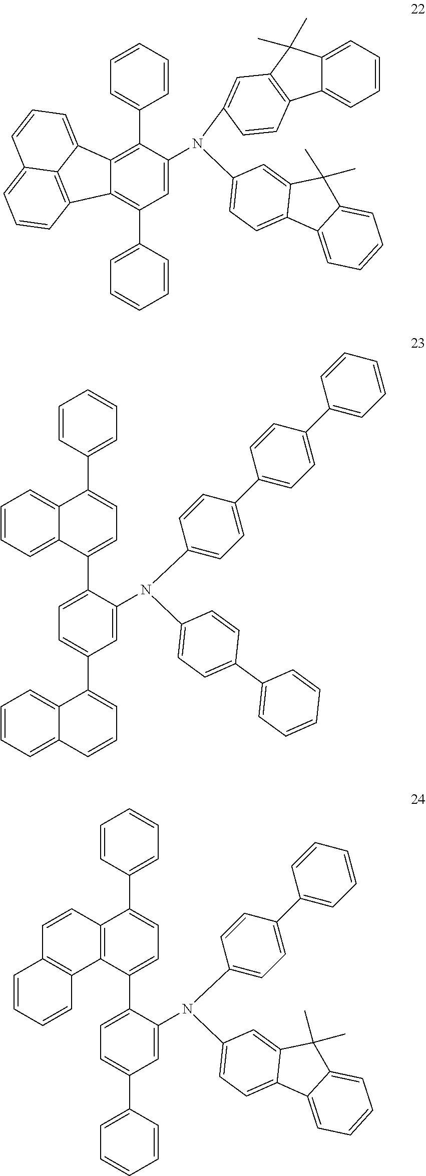 [SMILES: CC1(C)c2ccccc2-c2ccc(N(c3ccc4c(c3)C(C)(C)c3ccccc3-4)c3cc(-c4ccccc4)c4c(c3-c3ccccc3)-c3cccc5cccc-4c35)cc21 ; c1ccc(-c2ccc(-c3ccc(N(c4ccc(-c5ccccc5)cc4)c4cc(-c5cccc6ccccc56)ccc4-c4ccc(-c5ccccc5)c5ccccc45)cc3)cc2)cc1 ; CC1(C)c2ccccc2-c2ccc(N(c3ccc(-c4ccccc4)cc3)c3cc(-c4ccccc4)ccc3-c3ccc(-c4ccccc4)c4ccc5ccccc5c34)cc21]

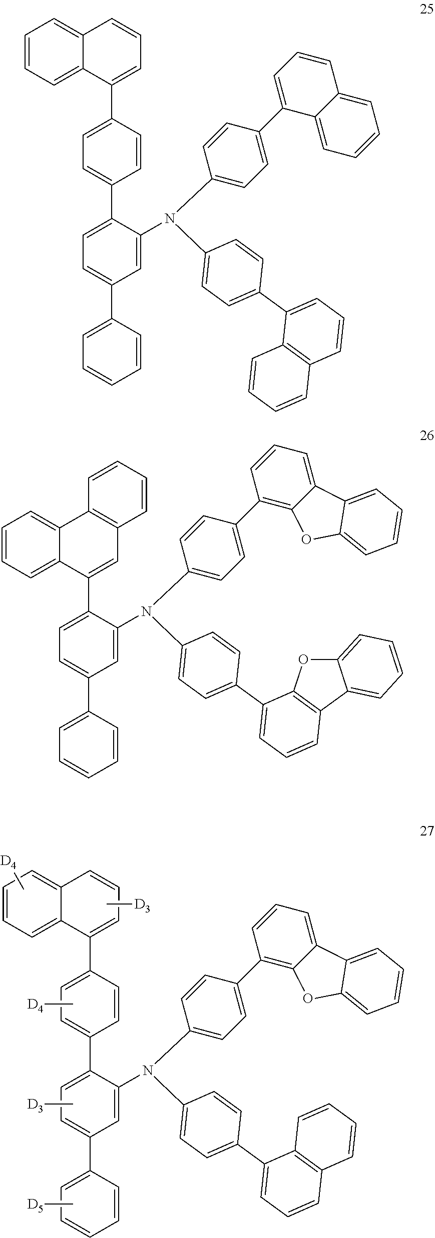

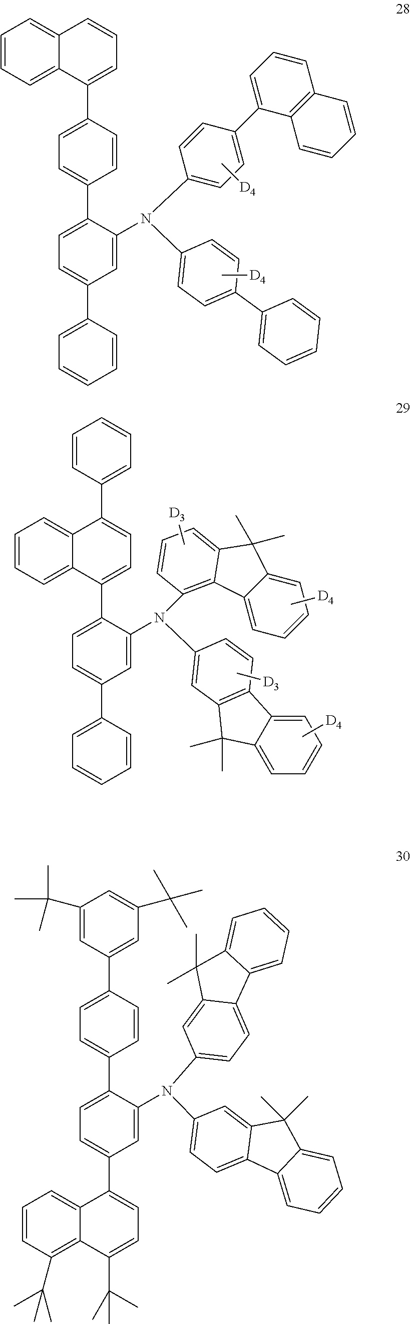

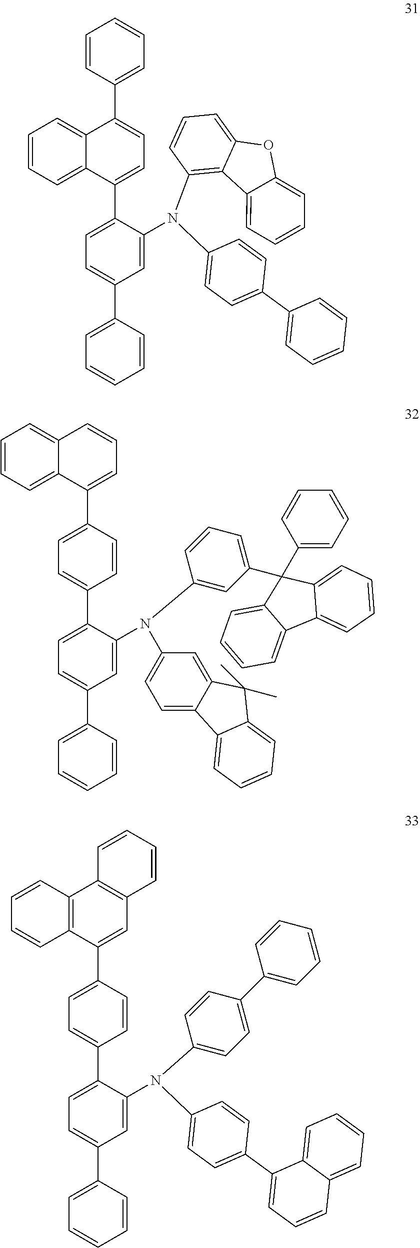 [SMILES: c1ccc(-c2ccc(N(c3cc(-c4ccccc4)ccc3-c3ccc(-c4ccccc4)c4ccccc34)c3cccc4oc5ccccc5c34)cc2)cc1 ; CC1(C)c2ccccc2-c2ccc(N(c3cccc(C4(c5ccccc5)c5ccccc5-c5ccccc54)c3)c3cc(-c4ccccc4)ccc3-c3ccc(-c4cccc5ccccc45)cc3)cc21 ; c1ccc(-c2ccc(N(c3ccc(-c4cccc5ccccc45)cc3)c3cc(-c4ccccc4)ccc3-c3ccc(-c4cc5ccccc5c5ccccc45)cc3)cc2)cc1]

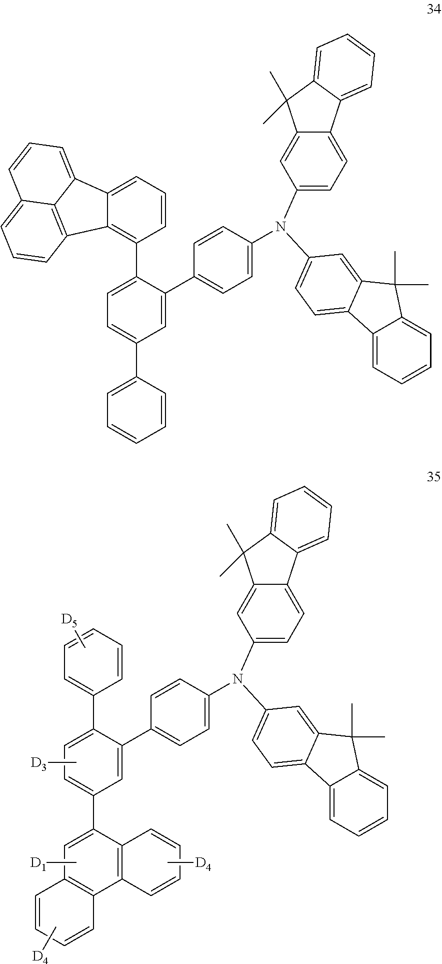

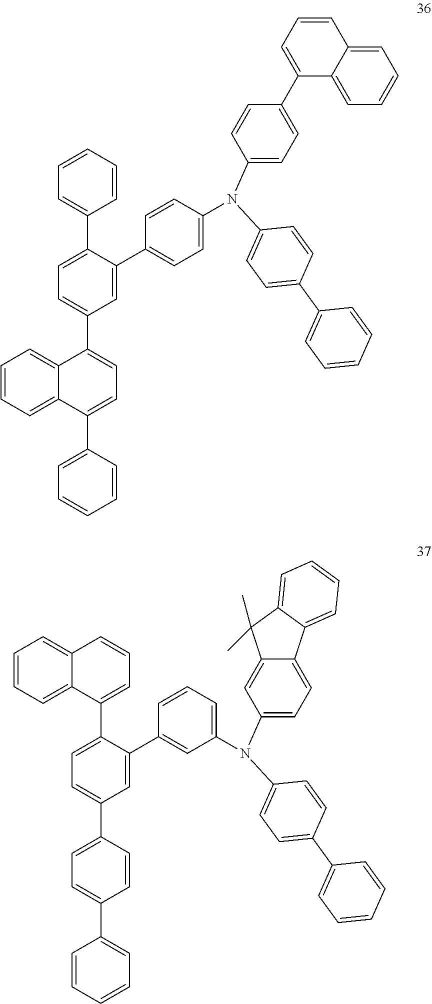 [SMILES: c1ccc(-c2ccc(N(c3ccc(-c4cc(-c5ccc(-c6ccccc6)c6ccccc56)ccc4-c4ccccc4)cc3)c3ccc(-c4cccc5ccccc45)cc3)cc2)cc1 ; CC1(C)c2ccccc2-c2ccc(N(c3ccc(-c4ccccc4)cc3)c3cccc(-c4cc(-c5ccc(-c6ccccc6)cc5)ccc4-c4cccc5ccccc45)c3)cc21]

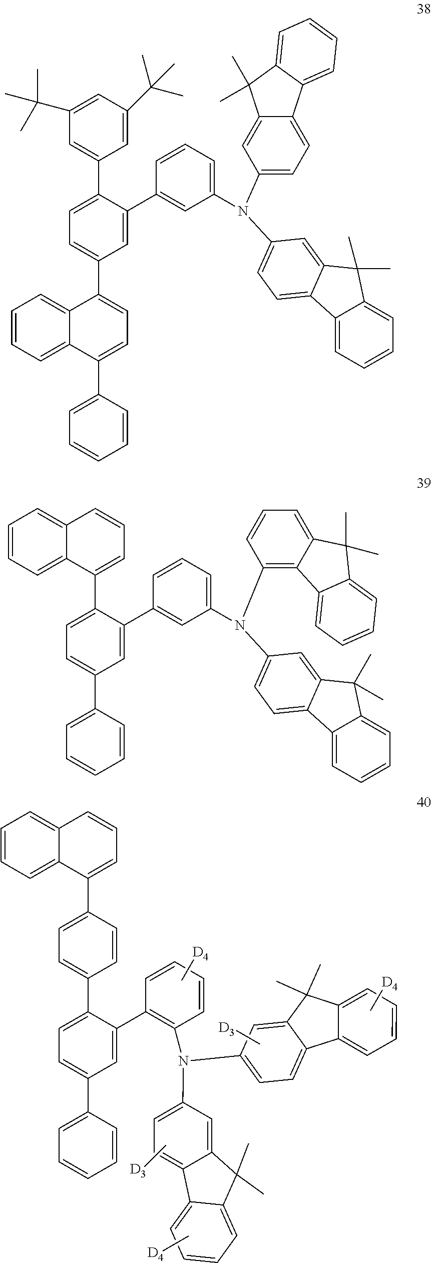

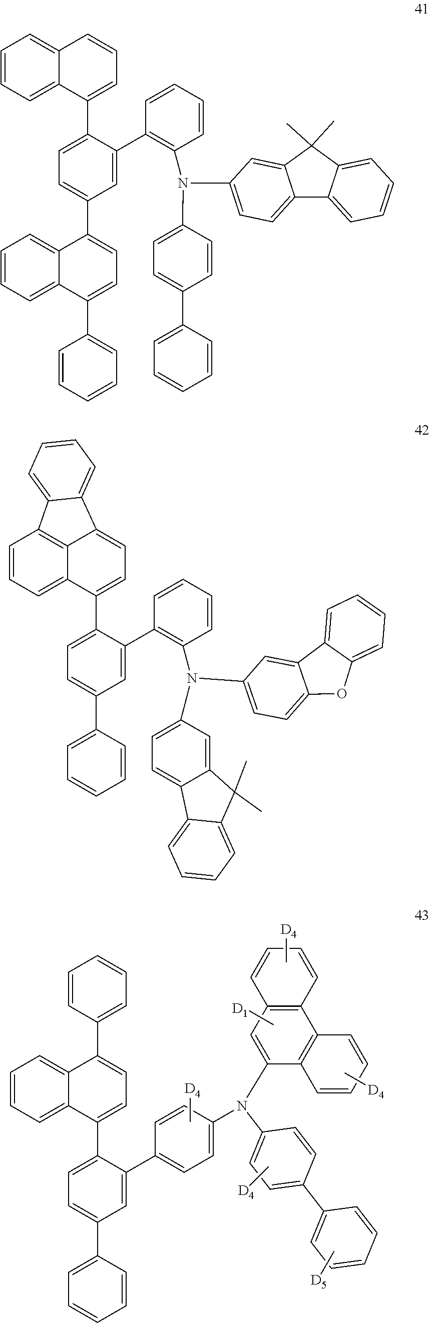

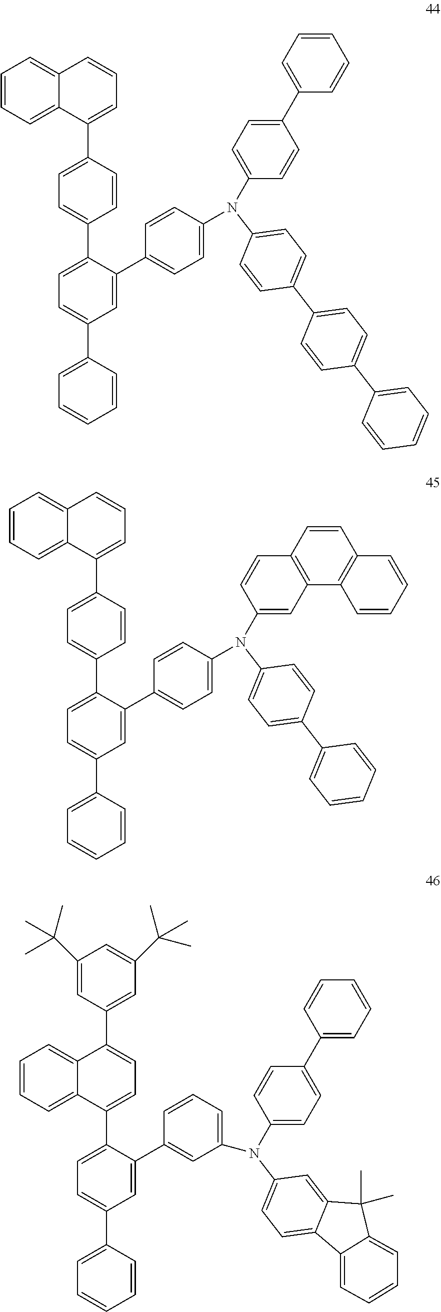 [SMILES: c1ccc(-c2ccc(-c3ccc(N(c4ccc(-c5ccccc5)cc4)c4ccc(-c5cc(-c6ccccc6)ccc5-c5ccc(-c6cccc7ccccc67)cc5)cc4)cc3)cc2)cc1 ; c1ccc(-c2ccc(N(c3ccc(-c4cc(-c5ccccc5)ccc4-c4ccc(-c5cccc6ccccc56)cc4)cc3)c3ccc4ccc5ccccc5c4c3)cc2)cc1 ; CC(C)(C)c1cc(-c2ccc(-c3ccc(-c4ccccc4)cc3-c3cccc(N(c4ccc(-c5ccccc5)cc4)c4ccc5c(c4)C(C)(C)c4ccccc4-5)c3)c3ccccc23)cc(C(C)(C)C)c1]

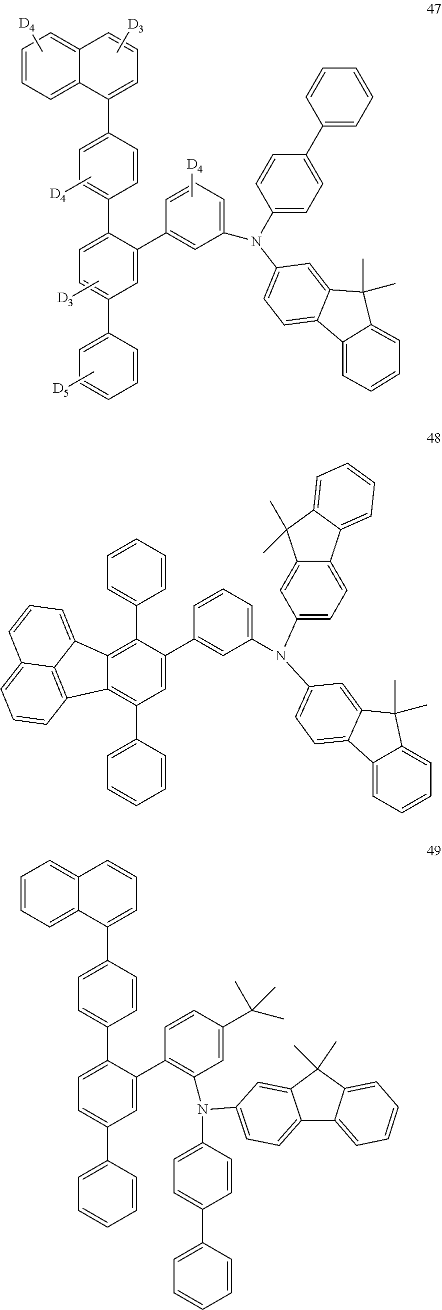

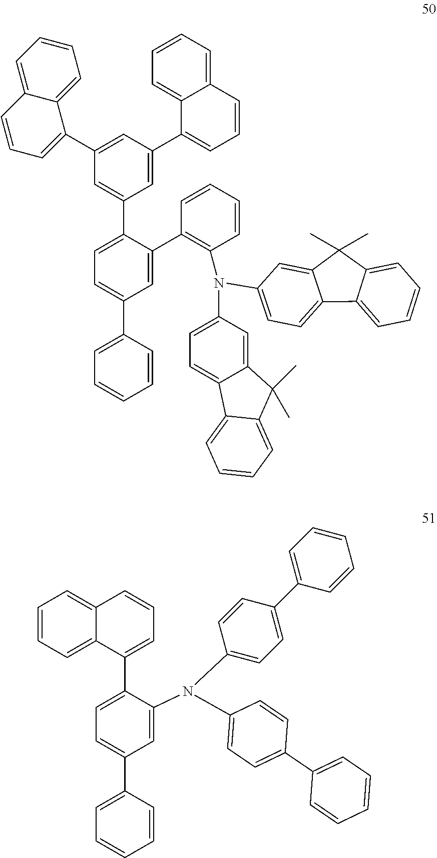 [SMILES: CC1(C)c2ccccc2-c2ccc(N(c3ccc4c(c3)C(C)(C)c3ccccc3-4)c3ccccc3-c3cc(-c4ccccc4)ccc3-c3cc(-c4cccc5ccccc45)cc(-c4cccc5ccccc45)c3)cc21 ; c1ccc(-c2ccc(N(c3ccc(-c4ccccc4)cc3)c3cc(-c4ccccc4)ccc3-c3cccc4ccccc34)cc2)cc1]

The compounds according to the present application may be prepared by means of the synthesis methods described hereinafter. The person skilled in the art will be able to modify and adapt the synthesis methods shown within the scope of their common art knowledge in order to obtain further compounds according to the application.

By the method shown in scheme 1, proceeding from an aryl-phenyl derivative substituted by two reactive groups, in a Suzuki coupling, it is possible to prepare a catenated trisaryl derivative substituted by a reactive group in a position ortho to the newly formed phenyl-aryl bond.

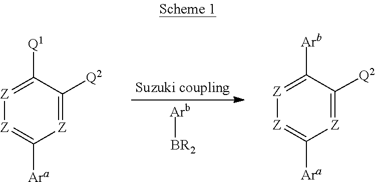

In a subsequent step, as shown in scheme 2, a Hartwig-Buchwald coupling can be effected, by means of which an amino group is introduced into the molecule in the position of the reactive group. This affords a compound according to the present application in which the index k=0.

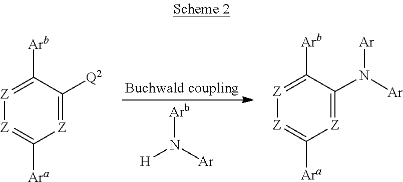

Alternatively, in a subsequent step, as shown in scheme 3, a Suzuki coupling can be effected in the position of the reactive group, by means of which an aromatic ring system containing an amino group is introduced into the molecule. This affords a compound according to the present application in which the index k>0.

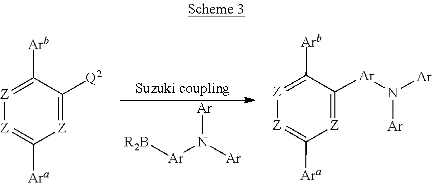

The definition of the variable groups in the schemes shown above is as follows:
Z═N or CR
R═H or organic radical
Q¹, Q²=reactive group
Ar, Ar^{a} and Ar^{b}=optionally substituted aromatic or heteroaromatic system

The compounds shown in the above method steps may have further substituents.

As an alternative to the compounds formed in the step of scheme 1, it is also possible to use commercially available compounds in the step of schemes 2 and 3 that have the corresponding chemical structures of the reactant from schemes 2 and 3.

The present application thus provides a process for preparing a compound according to the present application, characterized in that a catenated trisaryl derivative substituted by a reactive group is either a) reacted in a coupling reaction with a secondary amine, or b) reacted in a coupling reaction with an aromatic or heteroaromatic species having a boron-containing group and having an amino group.

The reactive group here is preferably selected from CI, Br and I, more preferably from Br and I. The coupling reaction in the reaction under a) is preferably a Hartwig-Buchwald coupling reaction. The coupling reaction under b) is preferably a Suzuki coupling reaction.

The catenated trisaryl derivative substituted by a reactive group is preferably prepared proceeding from an aryl-phenyl derivative substituted by two reactive groups by means of a Suzuki coupling reaction with an arylboronic acid compound.

The above-described compounds of the invention, especially compounds substituted by reactive leaving groups, such as bromine, iodine, chlorine, boronic acid or boronic ester, may find use as monomers for production of corresponding oligomers, dendrimers or polymers. Suitable reactive leaving groups are, for example, bromine, iodine, chlorine, boronic acids, boronic esters, amines, alkenyl or alkynyl groups having a terminal C≡C double bond or C—C triple bond, oxiranes, oxetanes, groups which enter into a cycloaddition, for example a 1,3-dipolar cycloaddition, for example dienes or azides, carboxylic acid derivatives, alcohols and silanes.

The invention therefore further provides oligomers, polymers or dendrimers containing one or more compounds of formula (I), wherein the bond(s) to the polymer, oligomer or dendrimer may be localized at any desired positions substituted by R¹, R², R³, R⁴ or R⁵ in formula (I). According to the linkage of the compound of formula (I), the compound is part of a side chain of the oligomer or polymer or part of the main chain.

Further disclosure relating to oligomers, polymers or dendrimers can be found at page 49 line 26-page 51 line 17 of WO 2020/109434 A1. The disclosure of these text passages is hereby fully incorporated into the present application by citation.

For the processing of the compounds of the invention from a liquid phase, for example by spin-coating or by printing methods, formulations of the compounds of the invention are required. These formulations may, for example, be solutions, dispersions or emulsions. For this purpose, it may be preferable to use mixtures of two or more solvents. Suitable and preferred solvents are, for example, toluene, anisole, o-, m- or p-xylene, methyl benzoate, mesitylene, tetralin, veratrole, THF, methyl-THF, THP, chlorobenzene, dioxane, phenoxytoluene, especially 3-phenoxytoluene, (-)-fenchone, 1,2,3,5-tetramethylbenzene, 1,2,4,5-tetramethylbenzene, 1-methylnaphthalene, 2-methylbenzothiazole, 2-phenoxyethanol, 2-pyrrolidinone, 3-methylanisole, 4-methylanisole, 3,4-dimethylanisole, 3,5-dimethylanisole, acetophenone, alpha-terpineol, benzothiazole, butyl benzoate, cumene, cyclohexanol, cyclohexanone, cyclohexylbenzene, decalin, dodecylbenzene, ethyl benzoate, indane, methyl benzoate, NMP, p-cymene, phenetole, 1,4-diisopropylbenzene, dibenzyl ether, diethylene glycol butyl methyl ether, triethylene glycol butyl methyl ether, diethylene glycol dibutyl ether, triethylene glycol dimethyl ether, diethylene glycol monobutyl ether, tripropylene glycol dimethyl ether, tetraethylene glycol dimethyl ether, 2-isopropylnaphthalene, pentylbenzene, hexylbenzene, heptylbenzene, octylbenzene, 1,1-bis(3,4-dimethylphenyl) ethane, or mixtures of these solvents.

The invention therefore further provides a formulation, especially a solution, dispersion or emulsion, comprising at least one compound of formula (I) or at least one polymer, oligomer or dendrimer containing at least one unit of formula (I) and at least one solvent, preferably an organic solvent. The way in which such solutions can be prepared is known to those skilled in the art.

The compound of formula (I) is suitable for use in an electronic device, especially an organic electroluminescent device (OLED). Depending on the substitution, the compound of the formula (I) can be used in different functions and layers. Preference is given to use as a hole-transporting material in a hole-transporting layer and/or as matrix material in an emitting layer, more preferably in combination with a phosphorescent emitter.

The invention therefore further provides for the use of a compound of formula (I) in an electronic device. This electronic device is preferably selected from the group consisting of organic integrated circuits (OICs), organic field-effect transistors (OFETs), organic thin-film transistors (OTFTs), organic light-emitting transistors (OLETs), organic solar cells (OSCs), organic optical detectors, organic photoreceptors, organic field-quench devices (OFQDs), organic light-emitting electrochemical cells (OLECs), organic laser diodes (O-lasers) and more preferably organic electroluminescent devices (OLEDs).

The invention further provides an electronic device comprising at least one compound of formula (I). This electronic device is preferably selected from the abovementioned devices.

Particular preference is given to an organic electroluminescent device comprising an anode, cathode and at least one emitting layer, characterized in that at least one organic layer comprising at least one compound of formula (I) is present in the device. Preference is given to an organic electroluminescent device comprising an anode, cathode and at least one emitting layer, characterized in that at least one organic layer in the device, selected from hole-transporting and emitting layers, comprises at least one compound of formula (I).

A hole-transporting layer is understood here to mean all layers disposed between anode and emitting layer, preferably hole injection layer, hole transport layer and electron blocker layer. A hole injection layer is understood here to mean a layer that directly adjoins the anode. A hole transport layer is understood here to mean a layer which is between the anode and emitting layer but does not directly adjoin the anode, and preferably does not directly adjoin the emitting layer either. An electron blocker layer is understood here to mean a layer which is between the anode and emitting layer and directly adjoins the emitting layer. An electron blocker layer preferably has a high-energy LUMO and hence prevents electrons from exiting from the emitting layer.

Apart from the cathode, anode and emitting layer, the electronic device may comprise further layers. These are selected, for example, from in each case one or more hole injection layers, hole transport layers, hole blocker layers, electron transport layers, electron injection layers, electron blocker layers, exciton blocker layers, interlayers, charge generation layers and/or organic or inorganic p/n junctions. However, it should be pointed out that not every one of these layers need necessarily be present and the choice of layers always depends on the compounds used and especially also on whether the device is a fluorescent or phosphorescent electroluminescent device.

The sequence of layers in the electronic device is preferably as follows:
anode-
hole injection layer-
hole transport layer-
optionally further hole transport layers-
emitting layer-
optionally hole blocker layer-
electron transport layer-
electron injection layer-
cathode-.

At the same time, it should be pointed out again that not all the layers mentioned need be present and/or that further layers may additionally be present.

The organic electroluminescent device of the invention may contain two or more emitting layers. More preferably, these emission layers have several emission maxima between 380 nm and 750 nm overall, such that the overall result is white emission; in other words, various emitting compounds which may fluoresce or phosphoresce and which emit blue, green, yellow, orange or red light are used in the emitting layers. Especially preferred are three-layer systems, i.e. systems having three emitting layers, wherein one of the three layers in each case shows blue emission, one of the three layers in each case shows green emission, and one of the three layers in each case shows orange or red emission. The compounds of the invention here are preferably present in a hole-transporting layer or in the emitting layer. It should be noted that, for the production of white light, rather than a plurality of color-emitting emitter compounds, an emitter compound used individually which emits over a broad wavelength range may also be suitable.

It is preferable that the compound of the formula (I) is used as hole transport material. The emitting layer here may be a fluorescent emitting layer, or it may be a phosphorescent emitting layer. The emitting layer is preferably a blue-fluorescing layer or a green-phosphorescing layer.

When the device containing the compound of the formula (I) contains a phosphorescent emitting layer, it is preferable that this layer contains two or more, preferably exactly two, different matrix materials (mixed matrix system). Preferred embodiments of mixed matrix systems are described in detail further down.

If the compound of formula (I) is used as hole transport material in a hole transport layer, a hole injection layer or an electron blocker layer, the compound can be used as pure material, i.e. in a proportion of 100%, in the hole transport layer, or it can be used in combination with one or more further compounds.

In a preferred embodiment, a hole-transporting layer comprising the compound of the formula (I) additionally comprises one or more further hole-transporting compounds. These further hole-transporting compounds are preferably selected from triarylamine compounds, more preferably from monotriarylamine compounds. They are most preferably selected from the preferred embodiments of hole transport materials that are specified further down. In the preferred embodiment described, the compound of the formula (I) and the one or more further hole-transporting compounds are preferably each present in a proportion of at least 10%, more preferably each in a proportion of at least 20%.

In a preferred embodiment, a hole-transporting layer comprising the compound of the formula (I) additionally contains one or more p-dopants. p-Dopants used according to the present invention are preferably those organic electron acceptor compounds capable of oxidizing one or more of the other compounds in the mixture.

Particularly preferred p-dopants are quinodimethane compounds, azaindenofluorenediones, azaphenalenes, azatriphenylenes, 12, metal halides, preferably transition metal halides, metal oxides, preferably metal oxides containing at least one transition metal or a metal of main group 3, and transition metal complexes, preferably complexes of Cu, Co, Ni, Pd and Pt with ligands containing at least one oxygen atom as bonding site. Preference is further given to transition metal oxides as dopants, preferably oxides of rhenium, molybdenum and tungsten, more preferably Re₂O₇, MoO₃, WO₃ and ReO₃. Still further preference is given to complexes of bismuth in the (III) oxidation state, more particularly bismuth (III) complexes with electron-deficient ligands, more particularly carboxylate ligands.

The p-dopants are preferably in substantially homogeneous distribution in the p-doped layers. This can be achieved, for example, by co-evaporation of the p-dopant and the hole transport material matrix. The p-dopant is preferably present in a proportion of 1% to 10% in the p-doped layer.

Preferred p-dopants are also the compounds depicted explicitly on pages 86-87 of published specification WO2021/156323A1.

In a preferred embodiment, a hole injection layer that conforms to one of the following embodiments is present in the device: a) it contains a triarylamine and a p-dopant; or b) it contains a single electron-deficient material (electron acceptor). In a preferred embodiment of embodiment a), the triarylamine is a monotriarylamine, especially one of the preferred triarylamine derivatives mentioned further down. In a preferred embodiment of embodiment b), the electron-deficient material is a hexaazatriphenylene derivative as described in US 2007/0092755.

The compound of the formula (I) may be present in a hole injection layer, in a hole transport layer and/or in an electron blocker layer of the device. When the compound is present in a hole injection layer or in a hole transport layer, it has preferably been p-doped, meaning that it is in mixed form with a p-dopant, as described above, in the layer.

The compound of the formula (I) is preferably present in an electron blocker layer. In this case, it is preferably not p-doped. Further preferably, in this case, it is preferably in the form of a single compound in the layer without addition of a further compound.

In an alternative preferred embodiment, the compound of the formula (I) is used in an emitting layer as matrix material in combination with one or more emitting compounds, preferably phosphorescent emitting compounds. The phosphorescent emitting compounds here are preferably selected from red-phosphorescing and green-phosphorescing compounds.

The proportion of the matrix material in the emitting layer in this case is between 50.0% and 99.9% by volume, preferably between 80.0% and 99.5% by volume, and more preferably between 85.0% and 97.0% by volume.

Correspondingly, the proportion of the emitting compound is between 0.1% and 50.0% by volume, preferably between 0.5% and 20.0% by volume, and more preferably between 3.0% and 15.0% by volume.

An emitting layer of an organic electroluminescent device may also contain systems comprising a plurality of matrix materials (mixed matrix systems) and/or a plurality of emitting compounds. In this case too, the emitting compounds are generally those compounds having the smaller proportion in the system and the matrix materials are those compounds having the greater proportion in the system. In individual cases, however, the proportion of a single matrix material in the system may be less than the proportion of a single emitting compound.

It is preferable that the compounds of formula (I) are used as a component of mixed matrix systems, preferably for phosphorescent emitters. The mixed matrix systems preferably comprise two or three different matrix materials, more preferably two different matrix materials. Preferably, in this case, one of the two materials is a material having hole-transporting properties and the other material is a material having electron-transporting properties. It is further preferable when one of the materials is selected from compounds having a large energy differential between HOMO and LUMO (wide-bandgap materials). The compound of the formula (1) in a mixed matrix system is preferably the matrix material having hole-transporting properties. Correspondingly, when the compound of the formula (I) is used as matrix material for a phosphorescent emitter in the emitting layer of an OLED, a second matrix compound having electron-transporting properties is present in the emitting layer. The two different matrix materials may be present here in a ratio of 1:50 to 1:1, preferably 1:20 to 1:1, more preferably 1:10 to 1:1 and most preferably 1:4 to 1:1.

In a preferred embodiment, in the case of mixed matrix systems, the two or more matrix materials present in the mixed matrix system, at least one of which preferably conforms to one of the formulae (I), are used as a mixture and applied by evaporation.

The desired electron-transporting and hole-transporting properties of the mixed matrix components may, however, also be combined mainly or entirely in a single mixed matrix component, in which case the further mixed matrix component(s) fulfill(s) other functions.

Preference is given to using the following material classes in the abovementioned layers of the device:

### Phosphorescent Emitters:

The term “phosphorescent emitters” typically encompasses compounds where the emission of light is effected through a spin-forbidden transition, for example a transition from an excited triplet state or a state having a higher spin quantum number, for example a quintet state.

Suitable phosphorescent emitters are especially compounds which, when suitably excited, emit light, preferably in the visible region, and also contain at least one atom of atomic number greater than 20, preferably greater than 38, and less than 84, more preferably greater than 56 and less than 80.

Preference is given to using, as phosphorescent emitters, compounds containing copper, molybdenum, tungsten, rhenium, ruthenium, osmium, rhodium, iridium, palladium, platinum, silver, gold or europium, especially compounds containing iridium, platinum or copper.

In the context of the present invention, all luminescent iridium, platinum or copper complexes are considered to be phosphorescent compounds.

In general, all phosphorescent complexes as used for phosphorescent OLEDs according to the prior art and as known to those skilled in the art in the field of organic electroluminescent devices are suitable for use in the devices of the invention. Further examples of suitable phosphorescent emitters are shown in the following table:

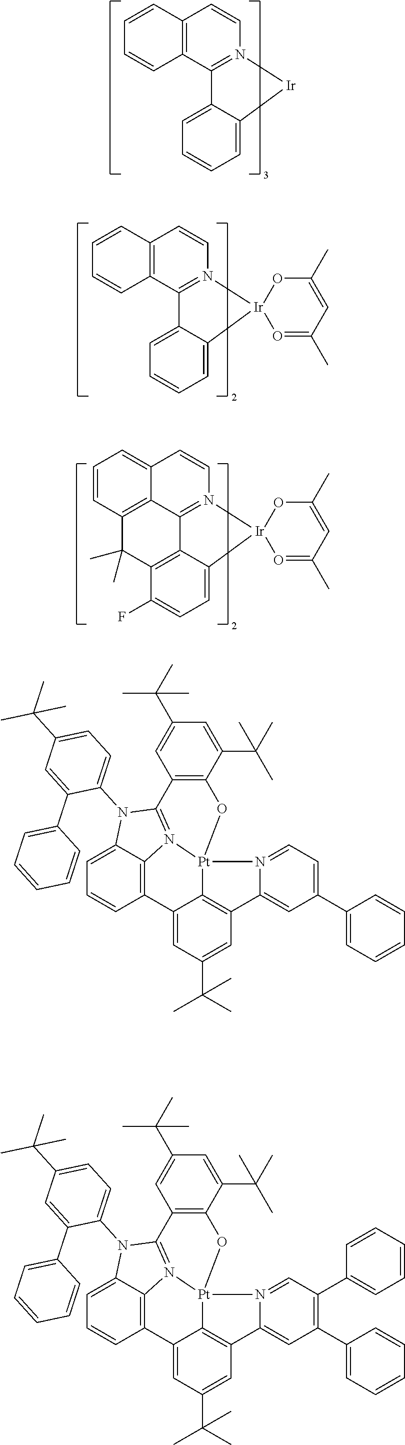

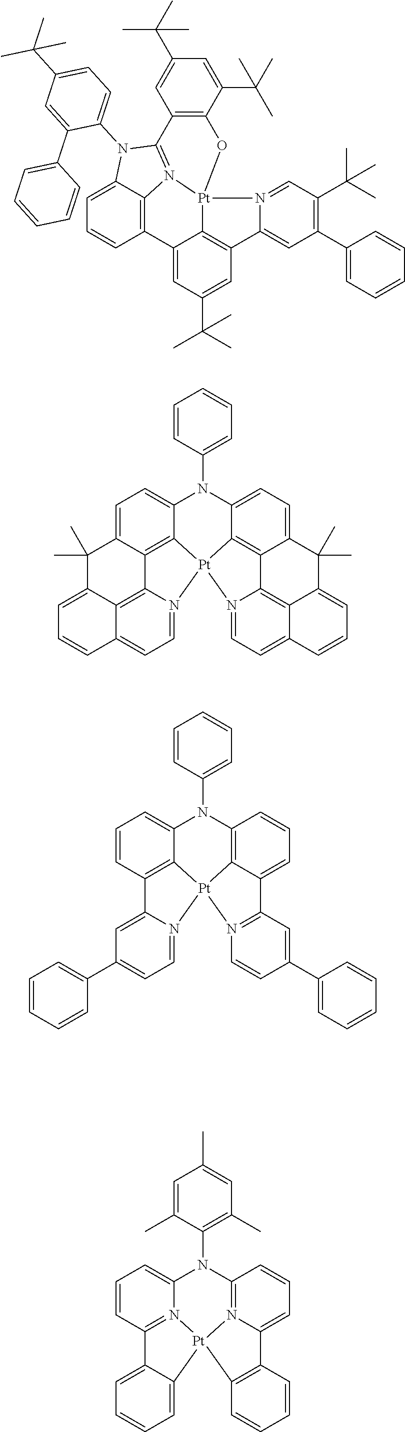

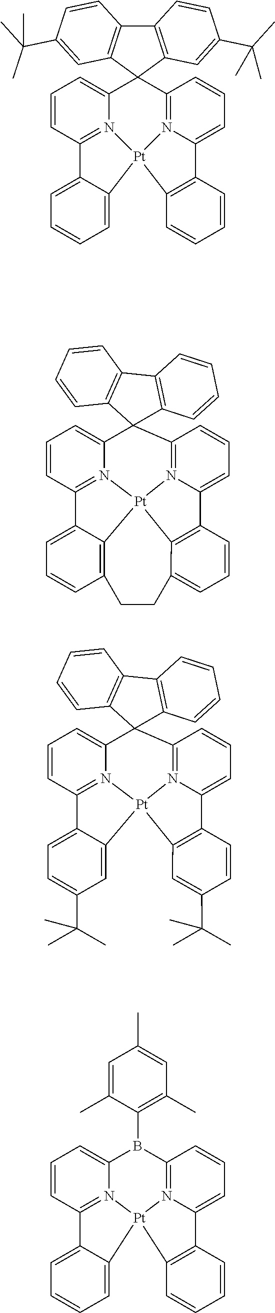

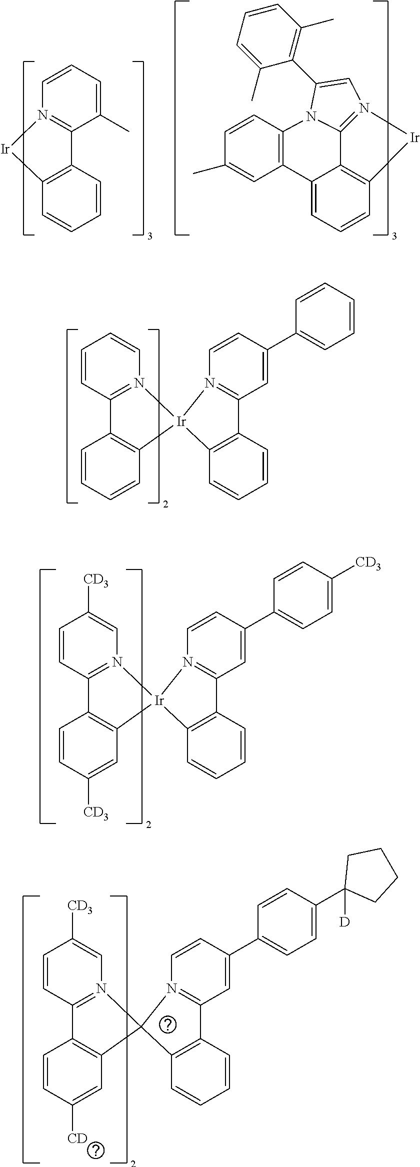

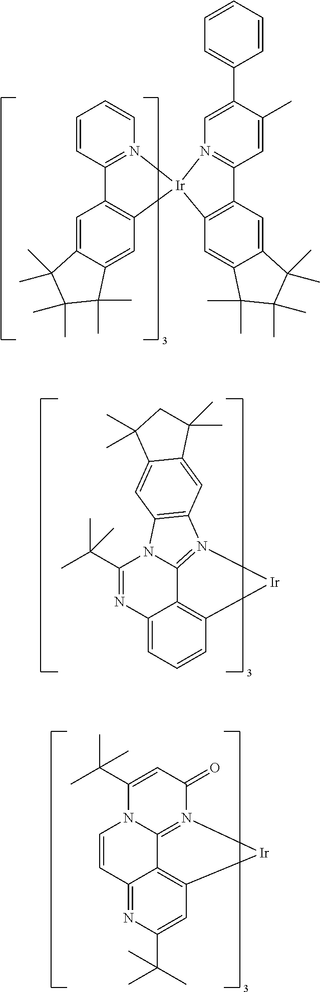

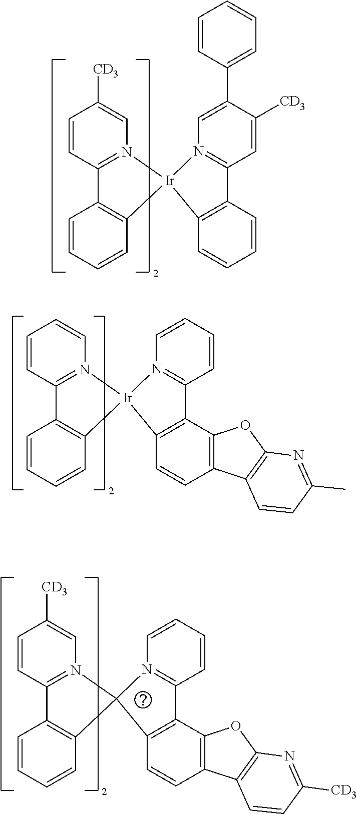

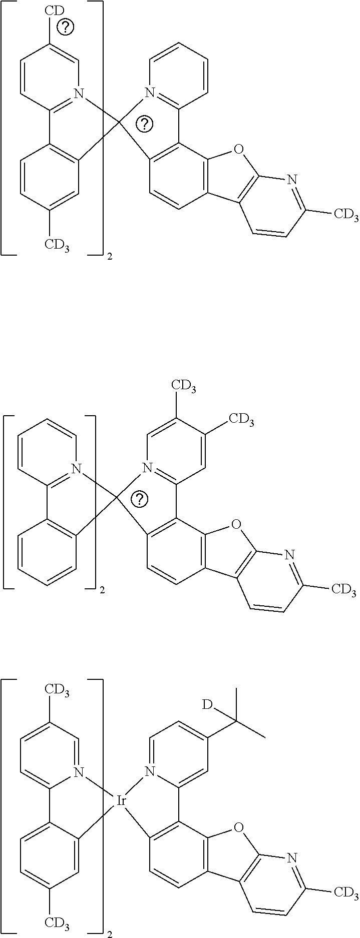

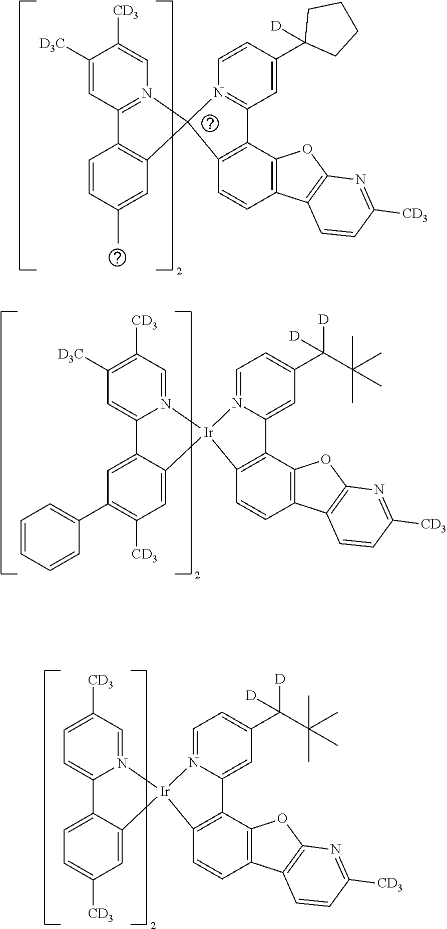

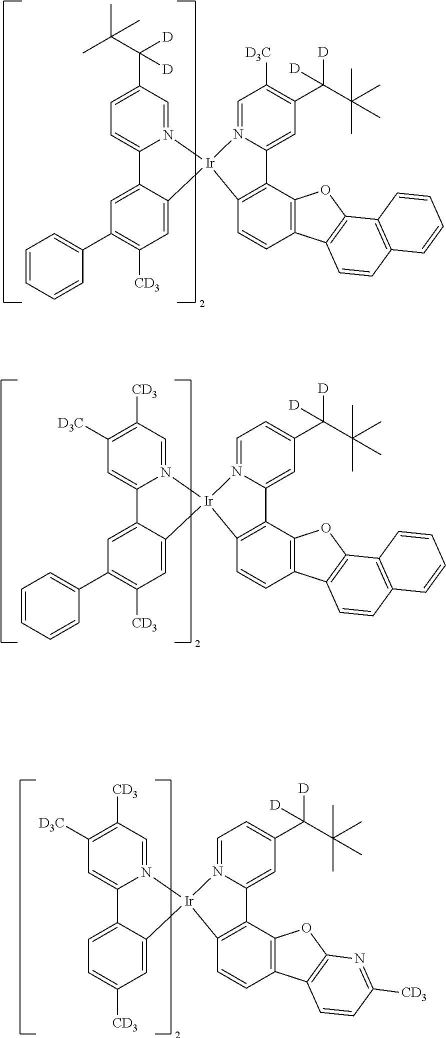

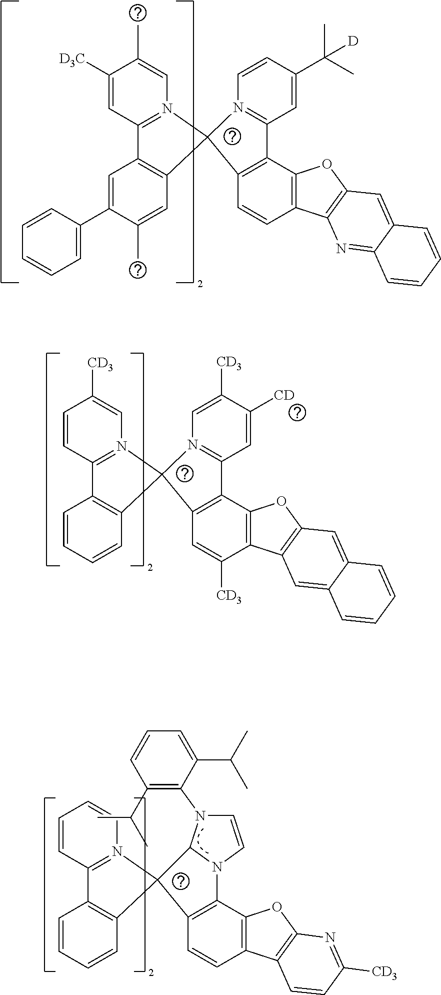

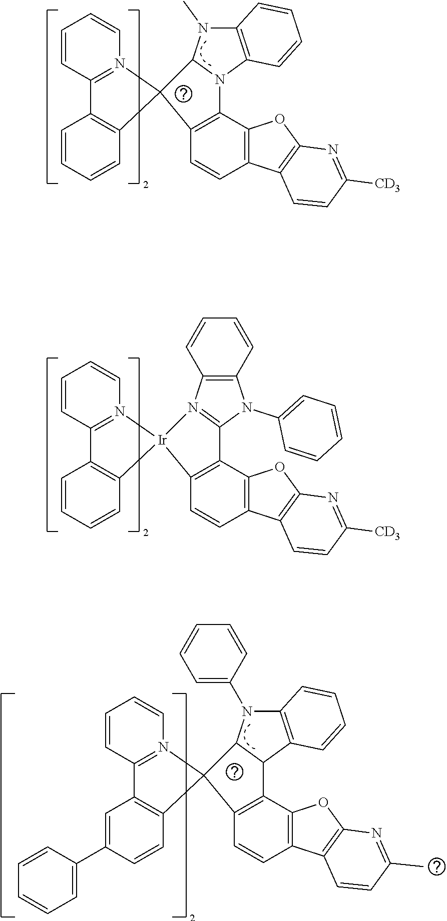

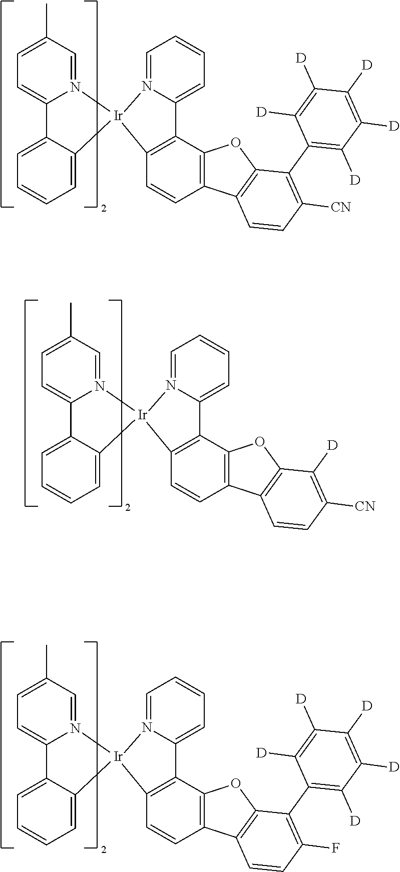

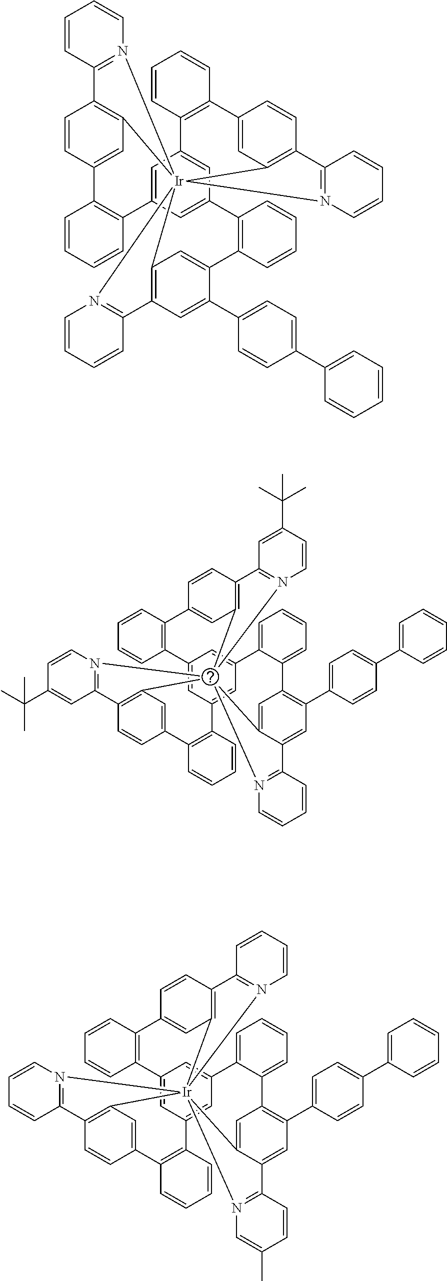

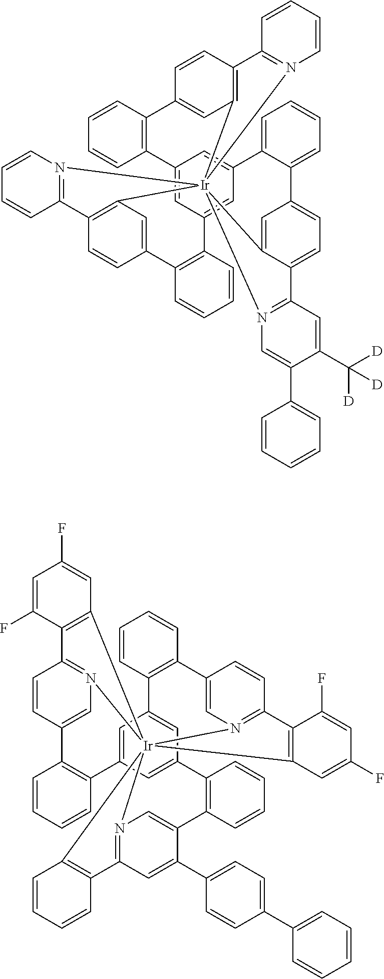

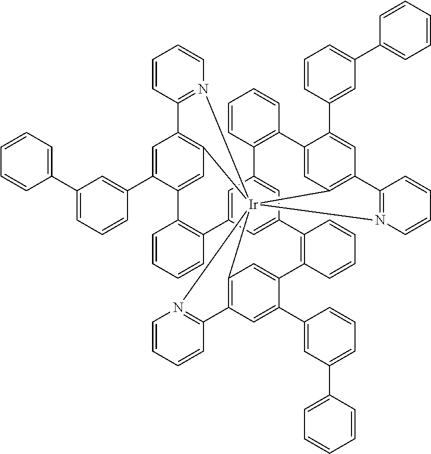

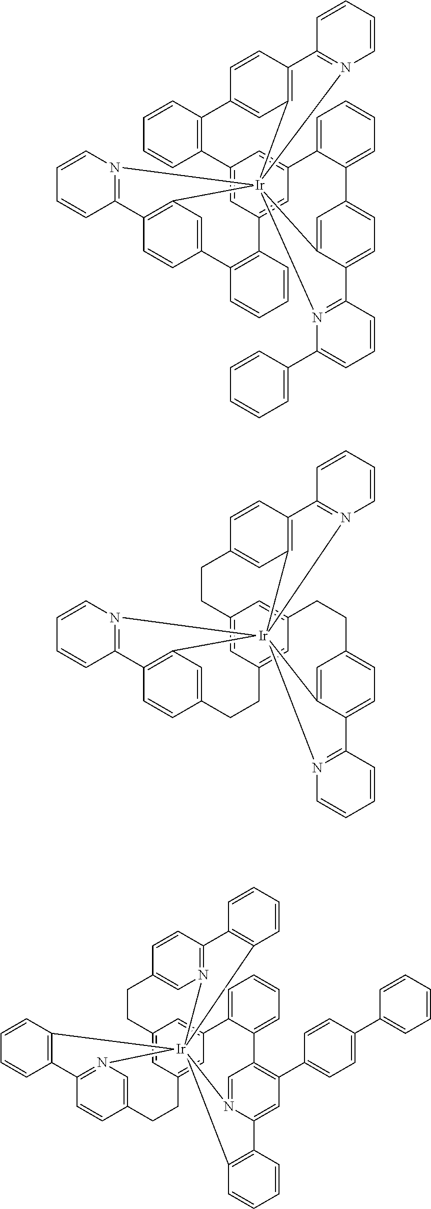

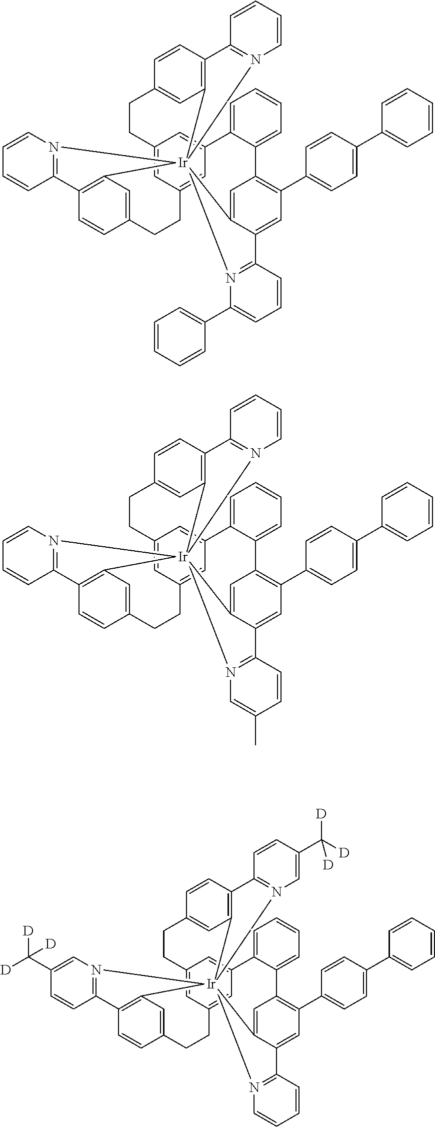

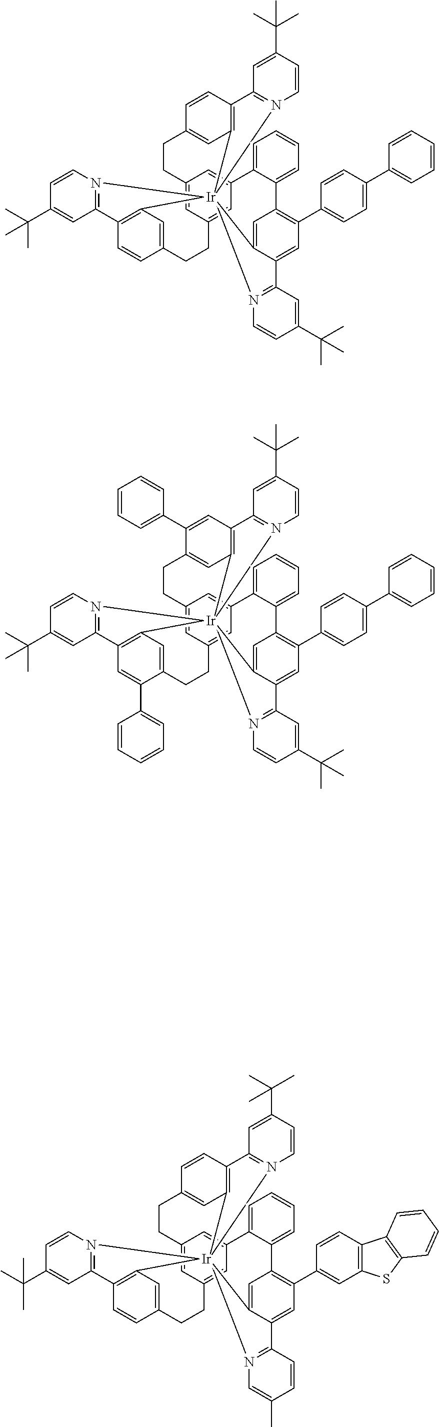

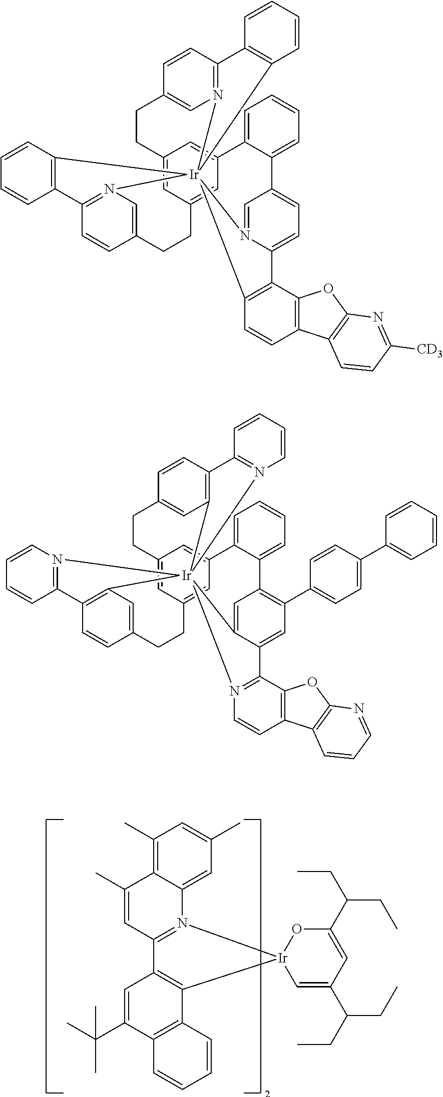

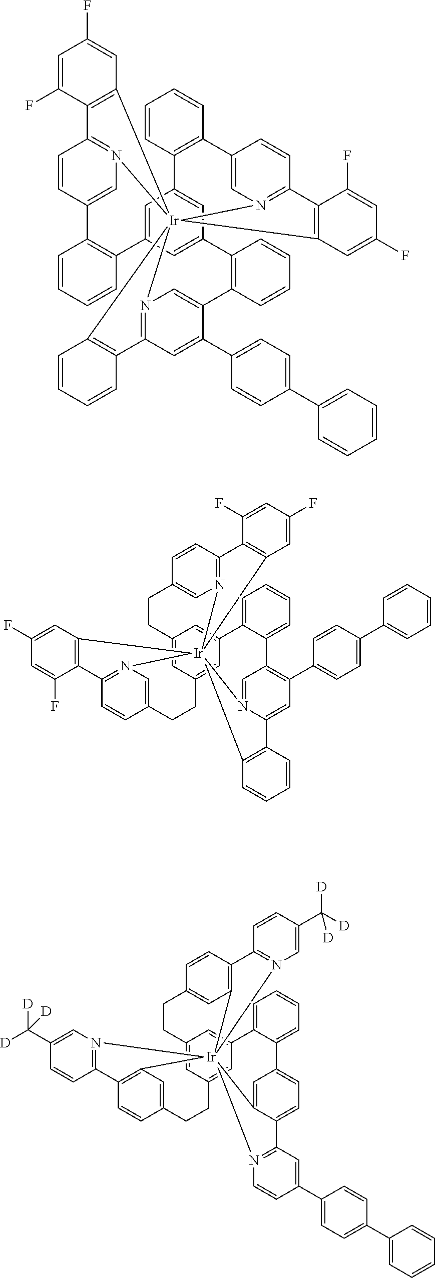

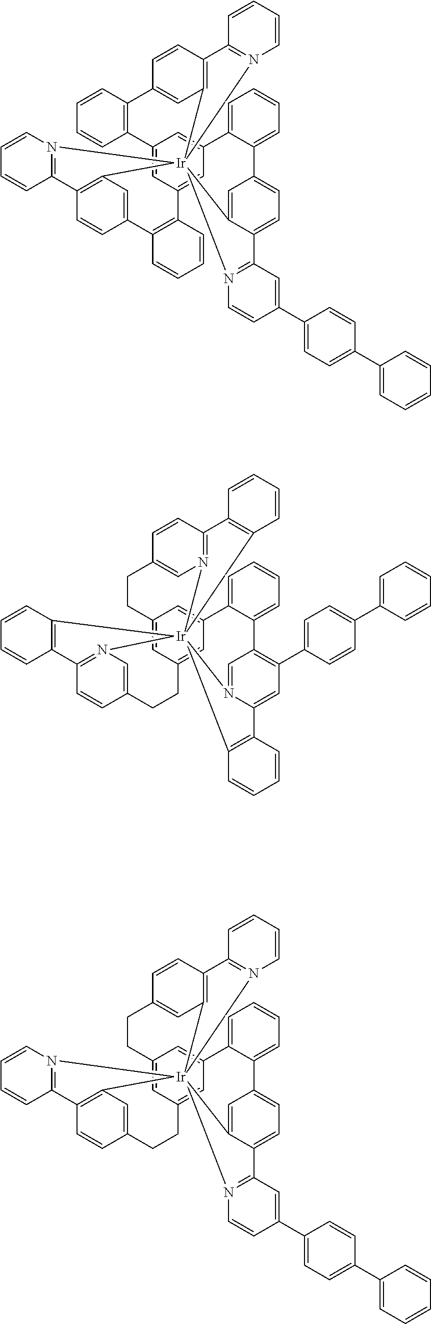

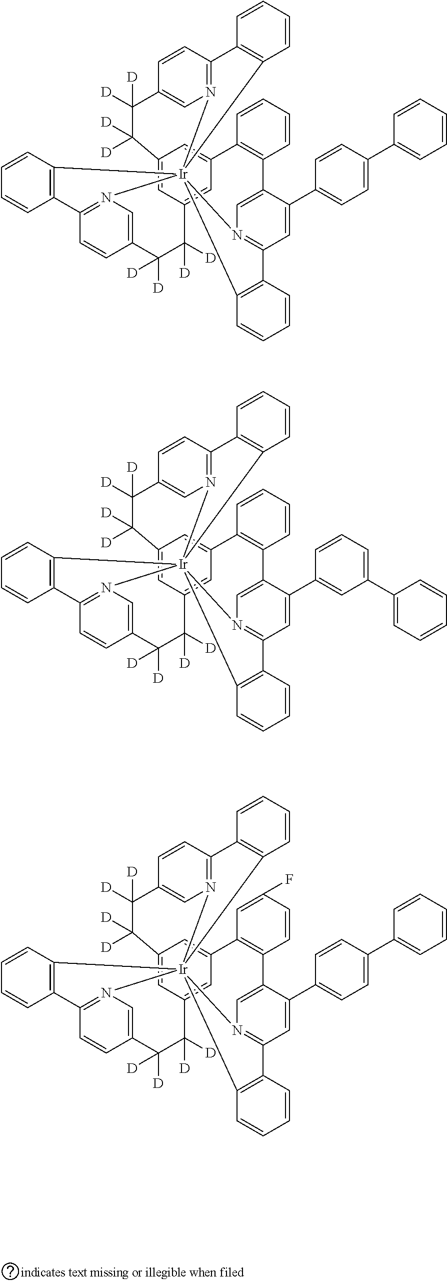

### Fluorescent Emitters:

Preferred fluorescent emitting compounds are selected from the class of the arylamines. An arylamine or an aromatic amine in the context of this invention is understood to mean a compound containing three substituted or unsubstituted aromatic or heteroaromatic ring systems bonded directly to the nitrogen. Preferably, at least one of these aromatic or heteroaromatic ring systems is a fused ring system, more preferably having at least 14 aromatic ring atoms. Preferred examples of these are aromatic anthraceneamines, aromatic anthracenediamines, aromatic pyreneamines, aromatic pyrenediamines, aromatic chryseneamines or aromatic chrysenediamines. What is meant by an aromatic anthraceneamine is a compound in which a diarylamino group is bonded directly to an anthracene group, preferably in the 9 position. What is meant by an aromatic anthracenediamine is a compound in which two diarylamino groups are bonded directly to an anthracene group, preferably in the 9,10 positions. Aromatic pyreneamines, pyrenediamines, chryseneamines and chrysenediamines are defined analogously, where the diarylamino groups are bonded to the pyrene preferably in the 1 position or 1,6 positions. Further preferred emitting compounds are indenofluoreneamines or -diamines, benzoindenofluoreneamines or -diamines, and dibenzoindenofluoreneamines or -diamines, and indenofluorene derivatives having fused aryl groups. Likewise preferred are pyrenearylamines. Likewise preferred are benzoindenofluoreneamines, benzofluoreneamines, extended benzoindenofluorenes, phenoxazines, and fluorene derivatives joined to furan units or to thiophene units.

### Matrix Materials for Fluorescent Emitters:

Preferred matrix materials for fluorescent emitters are selected from the classes of the oligoarylenes (e.g. 2,2′,7,7′-tetraphenylspirobifluorene), especially the oligoarylenes containing fused aromatic groups, the oligoarylenevinylenes, the polypodal metal complexes, the hole-conducting compounds, the electron-conducting compounds, especially ketones, phosphine oxides and sulfoxides; the atropisomers, the boronic acid derivatives or the benzanthracenes. Particularly preferred matrix materials are selected from the classes of the oligoarylenes comprising naphthalene, anthracene, benzanthracene and/or pyrene or atropisomers of these compounds, the oligoarylenevinylenes, the ketones, the phosphine oxides and the sulfoxides. Very particularly preferred matrix materials are selected from the classes of the oligoarylenes comprising anthracene, benzanthracene, benzophenanthrene and/or pyrene or atropisomers of these compounds. An oligoarylene in the context of this invention shall be understood to mean a compound in which at least three aryl or arylene groups are bonded to one another.

### Matrix Materials for Phosphorescent Emitters:

Preferred matrix materials for phosphorescent emitters are, as well as the compounds of the formula (I), aromatic ketones, aromatic phosphine oxides or aromatic sulfoxides or sulfones, triarylamines, carbazole derivatives, e.g. CBP (N,N-biscarbazolylbiphenyl) or carbazole derivatives, indolocarbazole derivatives, indenocarbazole derivatives, azacarbazole derivatives, bipolar matrix materials, silanes, azaboroles or boronic esters, triazine derivatives, zinc complexes, diazasilole or tetraazasilole derivatives, diazaphosphole derivatives, bridged carbazole derivatives, triphenylene derivatives, or lactams.

### Electron-Transporting Materials:

Suitable electron-transporting materials are, for example, the compounds disclosed in Y. Shirota et al., Chem. Rev. 2007, 107 (4), 953-1010, or other materials used in these layers according to the prior art.

Materials used for the electron transport layer may be any materials that are used as electron transport materials in the electron transport layer according to the prior art. Especially suitable are aluminum complexes, for example Alq₃, zirconium complexes, for example Zrq₄, lithium complexes, for example Liq, benzimidazole derivatives, triazine derivatives, pyrimidine derivatives, pyridine derivatives, pyrazine derivatives, quinoxaline derivatives, quinoline derivatives, oxadiazole derivatives, aromatic ketones, lactams, boranes, diazaphosphole derivatives and phosphine oxide derivatives.

Preferred electron transport and electron injection materials are also the compounds shown explicitly on pages 73-75 of WO2020/109434A1.

### Hole-Transporting Materials:

Further compounds which, in addition to the compounds of the formula (I), are preferably used in hole-transporting layers of the OLEDs of the invention are indenofluoreneamine derivatives, amine derivatives, hexaazatriphenylene derivatives, amine derivatives with fused aromatic systems, monobenzoindenofluoreneamines, dibenzoindenofluoreneamines, spirobifluoreneamines, fluoreneamines, spirodibenzopyranamines, dihydroacridine derivatives, spirodibenzofurans and spirodibenzothiophenes, phenanthrenediarylamines, spirotribenzotropolones, spirobifluorenes having meta-phenyldiamine groups, spirobisacridines, xanthenediarylamines, and 9,10-dihydroanthracene spiro compounds having diarylamino groups.

Preferred hole-transporting compounds are also the compounds depicted explicitly on pages 76-80 of WO2020/109434A1.

Preferred cathodes of the electronic device are metals having a low work function, metal alloys or multilayer structures composed of various metals, for example alkaline earth metals, alkali metals, main group metals or lanthanoids (e.g. Ca, Ba, Mg, Al, In, Mg, Yb, Sm, etc.). Additionally suitable are alloys composed of an alkali metal or alkaline earth metal and silver, for example an alloy composed of magnesium and silver. In the case of multilayer structures, in addition to the metals mentioned, it is also possible to use further metals having a relatively high work function, for example Ag or Al, in which case combinations of the metals such as Ca/Ag, Mg/Ag or Ba/Ag, for example, are generally used. It may also be preferable to introduce a thin interlayer of a material having a high dielectric constant between a metallic cathode and the organic semiconductor. Examples of useful materials for this purpose are alkali metal or alkaline earth metal fluorides, but also the corresponding oxides or carbonates (e.g. LiF, Li₂O, BaF₂, MgO, NaF, CsF, Cs₂CO₃, etc.). It is also possible to use lithium quinolinate (LiQ) for this purpose. The layer thickness of this layer is preferably between 0.5 and 5 nm.

Preferred anodes are materials having a high work function. Preferably, the anode has a work function of greater than 4.5 eV versus vacuum. Firstly, metals having a high redox potential are suitable for this purpose, for example Ag, Pt or Au. Secondly, metal/metal oxide electrodes (e.g. Al/Ni/NiOₓ, Al/PtOₓ) may also be preferred. For some applications, at least one of the electrodes has to be transparent or partly transparent in order to enable either the irradiation of the organic material (organic solar cell) or the outcoupling of light (OLED, O-LASER). Preferred anode materials here are conductive mixed metal oxides. Particular preference is given to indium tin oxide (ITO) or indium zinc oxide (IZO). Preference is further given to conductive doped organic materials, especially conductive doped polymers. In addition, the anode may also consist of two or more layers, for example of an inner layer of ITO and an outer layer of a metal oxide, preferably tungsten oxide, molybdenum oxide or vanadium oxide.

In a preferred embodiment, the electronic device is characterized in that one or more layers are coated by a sublimation process. In this case, the materials are applied by vapor deposition in vacuum sublimation systems at an initial pressure of less than 10⁻⁵ mbar, preferably less than 10⁻⁶ mbar. In this case, however, it is also possible that the initial pressure is even lower, for example less than 10⁻⁷ mbar.

Preference is likewise given to an electronic device, characterized in that one or more layers are coated by the OVPD (organic vapor phase deposition) method or with the aid of a carrier gas sublimation. In this case, the materials are applied at a pressure between 10⁻⁵ mbar and 1 bar. A special case of this method is the OVJP (organic vapor jet printing) method, in which the materials are applied directly by a nozzle and thus structured (for example M. S. Arnold et al., Appl. Phys. Lett. 2008, 92, 053301).

Preference is additionally given to an electronic device, characterized in that one or more layers are produced from solution, for example by spin-coating, or by any printing method, for example screen printing, flexographic printing, nozzle printing or offset printing, but more preferably LITI (light-induced thermal imaging, thermal transfer printing) or inkjet printing. For this purpose, soluble compounds of formula (I) are needed. High solubility can be achieved by suitable substitution of the compounds.

It is further preferable that an electronic device of the invention is produced by applying one or more layers from solution and one or more layers by a sublimation method.

After application of the layers, according to the use, the device is structured, contact-connected and finally sealed, in order to rule out damaging effects of water and air.

According to the invention, the electronic devices comprising one or more compounds of formula (I) can be used in displays, as light sources in lighting applications and as light sources in medical and/or cosmetic applications.

### EXAMPLES

### A) Synthesis Examples

### Synthesis of 3-bromo-4-[4-(naphthalen-1-yl)phenyl]-1,1′-biphenyl 1a

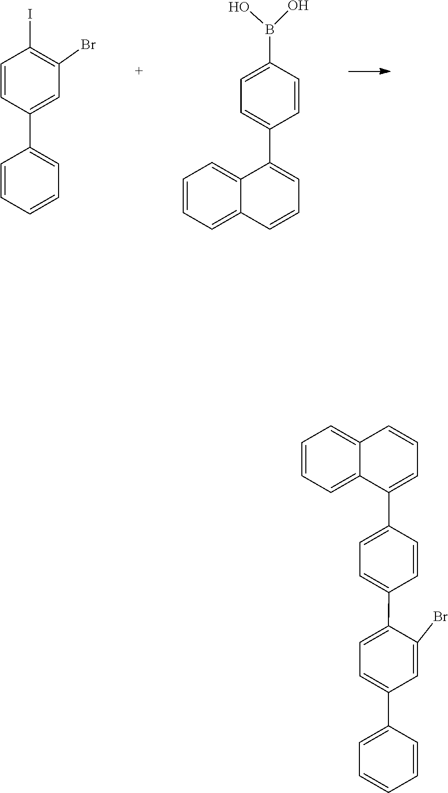 [SMILES: Brc1cc(-c2ccccc2)ccc1I ; OB(O)c1ccc(-c2cccc3ccccc23)cc1 ; Brc1cc(-c2ccccc2)ccc1-c1ccc(-c2cccc3ccccc23)cc1]

16.5 g (66.5 mmol) of [4-(naphthalen-1-yl)phenyl]boronic acid, 23.8 g (66.5 mmol) of 3-bromo-4-iodo-1,1′-biphenyl and 1.2 g (2 mmol) of bis(triphenylphosphine) Pd(II) chloride and 23 g (167 mmol) of potassium carbonate are suspended in 520 ml of acetonitrile and 220 ml of methanol. The reaction mixture is heated to boiling under a protective atmosphere overnight. The mixture is then filtered with suction, and the filtride is washed with MeOH, water and MeOH again. The residue is purified by crystallization with MeOH. Yield: 25 g (85% of theory), purity by GC-MS>94%.

The following compounds are prepared in an analogous manner:

| Ex. | Halogen derivative | Boronic acid | Product |
|---|---|---|---|
| 1b | 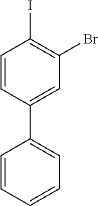 [SMILES: Brc1cc(-c2ccccc2)ccc1I] | 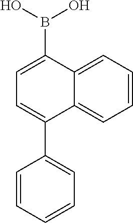 [SMILES: OB(O)c1ccc(-c2ccccc2)c2ccccc12] | 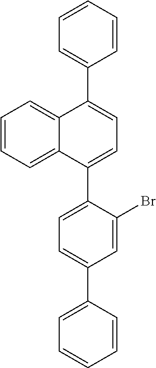 [SMILES: Brc1cc(-c2ccccc2)ccc1-c1ccc(-c2ccccc2)c2ccccc12] |
| 1c | 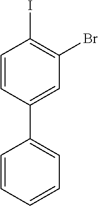 [SMILES: Brc1cc(-c2ccccc2)ccc1I] | 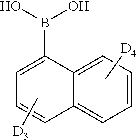 | 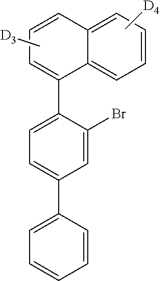 |
| 1d | 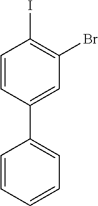 [SMILES: Brc1cc(-c2ccccc2)ccc1I] | 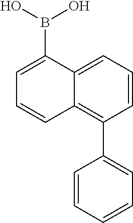 [SMILES: OB(O)c1cccc2c(-c3ccccc3)cccc12] | 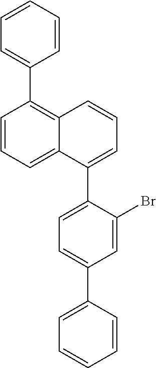 [SMILES: Brc1cc(-c2ccccc2)ccc1-c1cccc2c(-c3ccccc3)cccc12] |
| 1e | 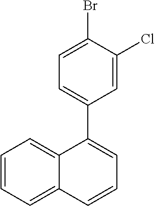 [SMILES: Clc1cc(-c2cccc3ccccc23)ccc1Br] | 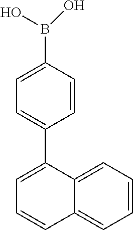 [SMILES: OB(O)c1ccc(-c2cccc3ccccc23)cc1] | 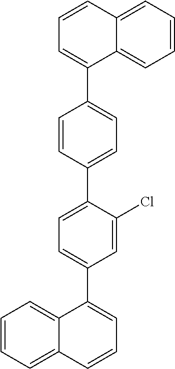 [SMILES: Clc1cc(-c2cccc3ccccc23)ccc1-c1ccc(-c2cccc3ccccc23)cc1] |
| 1f | 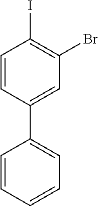 [SMILES: Brc1cc(-c2ccccc2)ccc1I] | 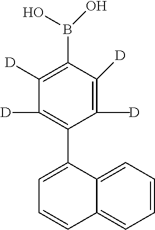 | 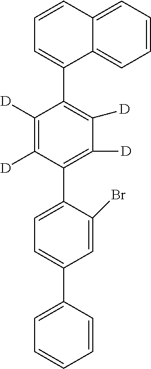 [SMILES: [2H]c1c([2H])c(-c2cccc3ccccc23)c([2H])c([2H])c1-c1ccc(-c2ccccc2)cc1Br] |
| 1g | 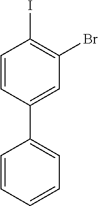 [SMILES: Brc1cc(-c2ccccc2)ccc1I] | 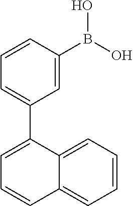 [SMILES: OB(O)c1cccc(-c2cccc3ccccc23)c1] | 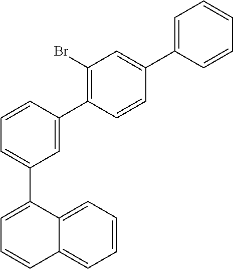 [SMILES: Brc1cc(-c2ccccc2)ccc1-c1cccc(-c2cccc3ccccc23)c1] |
| 1h | 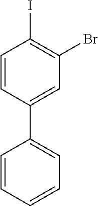 [SMILES: Brc1cc(-c2ccccc2)ccc1I] | 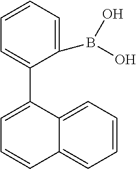 [SMILES: OB(O)c1ccccc1-c1cccc2ccccc12] | 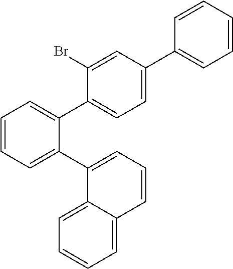 [SMILES: Brc1cc(-c2ccccc2)ccc1-c1ccccc1-c1cccc2ccccc12] |
| 1i | 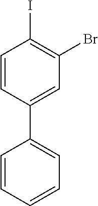 [SMILES: Brc1cc(-c2ccccc2)ccc1I] | 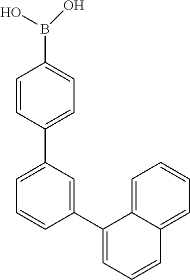 [SMILES: OB(O)c1ccc(-c2cccc(-c3cccc4ccccc34)c2)cc1] | 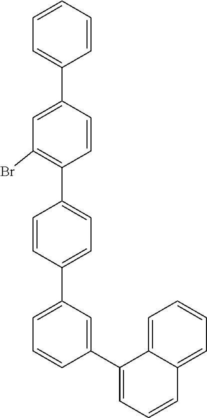 [SMILES: Brc1cc(-c2ccccc2)ccc1-c1ccc(-c2cccc(-c3cccc4ccccc34)c2)cc1] |
| 1j | 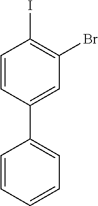 [SMILES: Brc1cc(-c2ccccc2)ccc1I] | 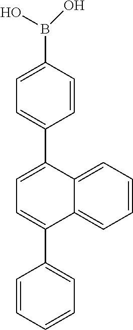 [SMILES: OB(O)c1ccc(-c2ccc(-c3ccccc3)c3ccccc23)cc1] | 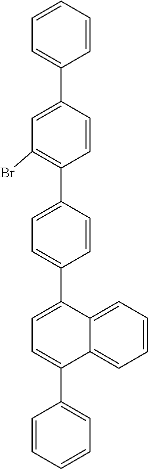 [SMILES: Brc1cc(-c2ccccc2)ccc1-c1ccc(-c2ccc(-c3ccccc3)c3ccccc23)cc1] |
| 1k | 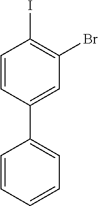 [SMILES: Brc1cc(-c2ccccc2)ccc1I] | 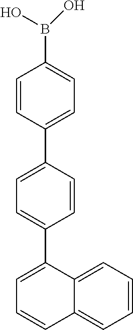 [SMILES: OB(O)c1ccc(-c2ccc(-c3cccc4ccccc34)cc2)cc1] | 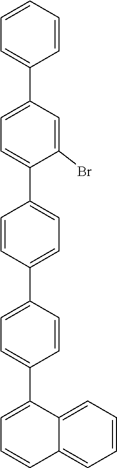 [SMILES: Brc1cc(-c2ccccc2)ccc1-c1ccc(-c2ccc(-c3cccc4ccccc34)cc2)cc1] |
| 1l | 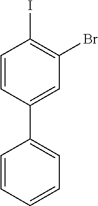 [SMILES: Brc1cc(-c2ccccc2)ccc1I] | 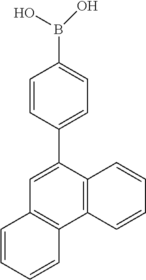 [SMILES: OB(O)c1ccc(-c2cc3ccccc3c3ccccc23)cc1] | 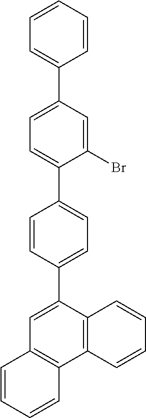 [SMILES: Brc1cc(-c2ccccc2)ccc1-c1ccc(-c2cc3ccccc3c3ccccc23)cc1] |
| 1m | 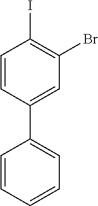 [SMILES: Brc1cc(-c2ccccc2)ccc1I] | 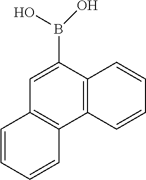 [SMILES: OB(O)c1cc2ccccc2c2ccccc12] | 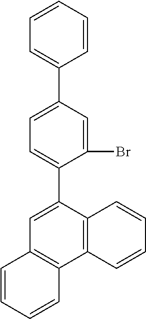 [SMILES: Brc1cc(-c2ccccc2)ccc1-c1cc2ccccc2c2ccccc12] |
| 1n | 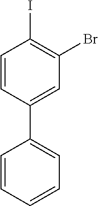 [SMILES: Brc1cc(-c2ccccc2)ccc1I] | 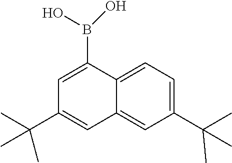 [SMILES: CC(C)(C)c1ccc2c(B(O)O)cc(C(C)(C)C)cc2c1] | 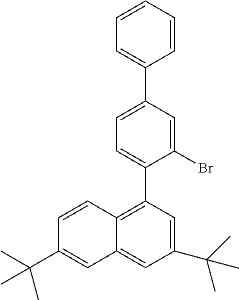 [SMILES: CC(C)(C)c1ccc2c(-c3ccc(-c4ccccc4)cc3Br)cc(C(C)(C)C)cc2c1] |
| 1o | 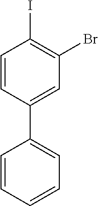 [SMILES: Brc1cc(-c2ccccc2)ccc1I] | 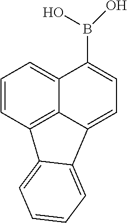 [SMILES: OB(O)c1ccc2c3c(cccc13)-c1ccccc1-2] | 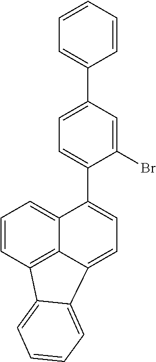 [SMILES: Brc1cc(-c2ccccc2)ccc1-c1ccc2c3c(cccc13)-c1ccccc1-2] |
| 1p | 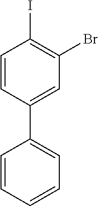 [SMILES: Brc1cc(-c2ccccc2)ccc1I] | 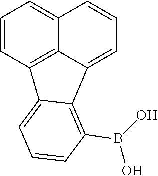 [SMILES: OB(O)c1cccc2c1-c1cccc3cccc-2c13] | 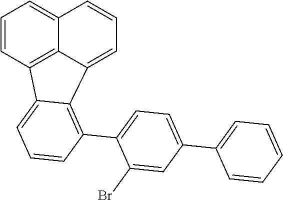 [SMILES: Brc1cc(-c2ccccc2)ccc1-c1cccc2c1-c1cccc3cccc-2c13] |

### Synthesis of N-(9,9-dimethyl-9H-fluoren-2-yl)-9,9-dimethyl-N-{4-[4-(naphthalen-1-yl)phenyl]-[1,1′-biphenyl]-3-yl}-9H-fluorene-2-amine 2a

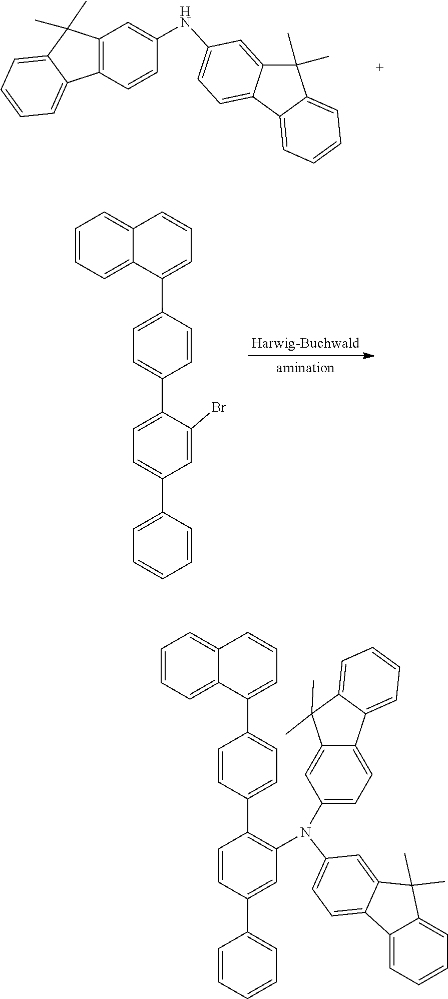 [SMILES: CC1(C)c2ccccc2-c2ccc(Nc3ccc4c(c3)C(C)(C)c3ccccc3-4)cc21 ; Brc1cc(-c2ccccc2)ccc1-c1ccc(-c2cccc3ccccc23)cc1 ; CC1(C)c2ccccc2-c2ccc(N(c3ccc4c(c3)C(C)(C)c3ccccc3-4)c3cc(-c4ccccc4)ccc3-c3ccc(-c4cccc5ccccc45)cc3)cc21]

N-(9,9-dimethyl-9H-fluoren-2-yl)-9,9-dimethyl-9H-fluorene-2-amine (32.3 g, 75 mmol), 3-bromo-4-[4-(naphthalen-1-yl)phenyl]-1, 1′-biphenyl, (29 g, 75 mmol) and sodium tert-butoxide (14.7 g, 150 mmol) are dissolved in 350 ml of toluene. The solution is degassed and saturated with N₂. Then tri-tert-butylphosphine (7.5 ml; 7.5 mmol, 1M in xylene) and 3.4 g (3.8 mmol) of Pd₂(dba)₃ are added thereto. The reaction mixture is heated to boiling under a protective atmosphere overnight. The mixture is cooled and partitioned between toluene and water and the organic phase is washed three times with water and dried over Na₂SO₄ and concentrated by rotary evaporation. After the crude product has been filtered through silica gel with toluene, the remaining residue is recrystallized from toluene and finally sublimed under high vacuum; purity is 99.9%. The yield is 23.9 g (42% of theory).

The following compounds are prepared in an analogous manner:

| Ex. | Halogen derivative | Amine | Product |
|---|---|---|---|
| 2b | 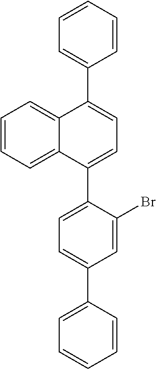 [SMILES: Brc1cc(-c2ccccc2)ccc1-c1ccc(-c2ccccc2)c2ccccc12] | 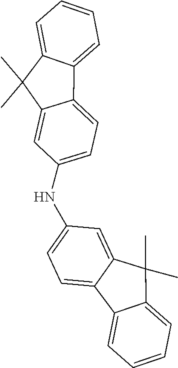 | 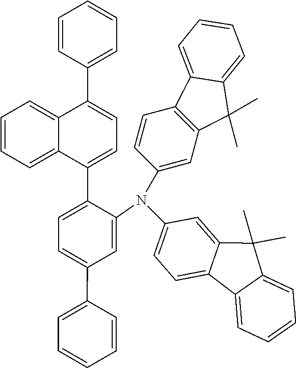 [SMILES: CC1(C)c2ccccc2-c2ccc(N(c3ccc4c(c3)C(C)(C)c3ccccc3-4)c3cc(-c4ccccc4)ccc3-c3ccc(-c4ccccc4)c4ccccc34)cc21] |
| 2c | 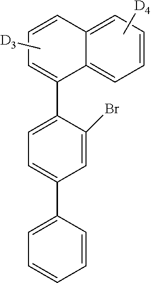 | 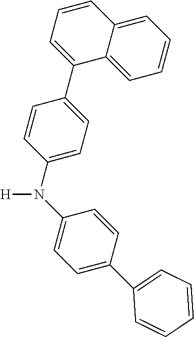 [SMILES: c1ccc(-c2ccc(Nc3ccc(-c4cccc5ccccc45)cc3)cc2)cc1] | 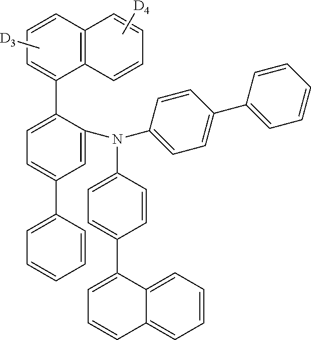 |
| 2d | 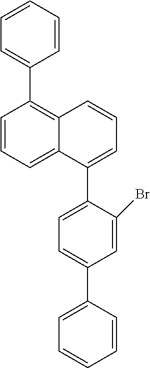 [SMILES: Brc1cc(-c2ccccc2)ccc1-c1cccc2c(-c3ccccc3)cccc12] | 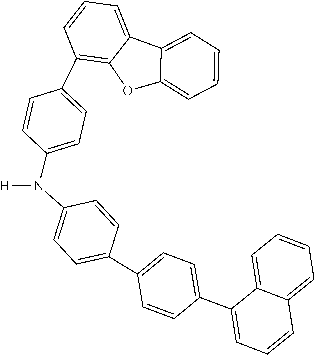 [SMILES: c1ccc2c(-c3ccc(-c4ccc(Nc5ccc(-c6cccc7c6oc6ccccc67)cc5)cc4)cc3)cccc2c1] | 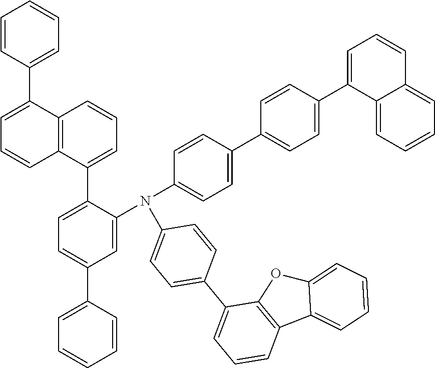 [SMILES: c1ccc(-c2ccc(-c3cccc4c(-c5ccccc5)cccc34)c(N(c3ccc(-c4ccc(-c5cccc6ccccc56)cc4)cc3)c3ccc(-c4cccc5c4oc4ccccc45)cc3)c2)cc1] |
| 2e | 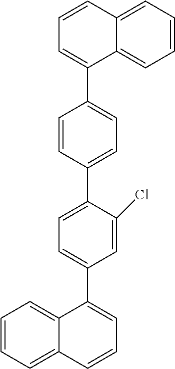 [SMILES: Clc1cc(-c2cccc3ccccc23)ccc1-c1ccc(-c2cccc3ccccc23)cc1] | 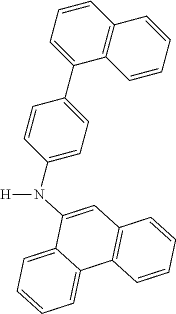 [SMILES: c1ccc2c(-c3ccc(Nc4cc5ccccc5c5ccccc45)cc3)cccc2c1] | 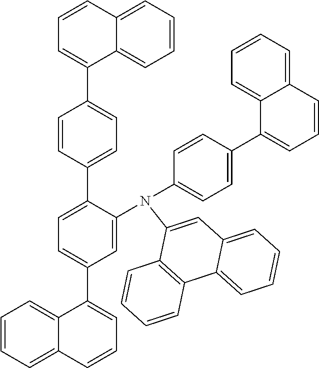 [SMILES: c1ccc2c(-c3ccc(-c4ccc(-c5cccc6ccccc56)cc4N(c4ccc(-c5cccc6ccccc56)cc4)c4cc5ccccc5c5ccccc45)cc3)cccc2c1] |
| 2f | 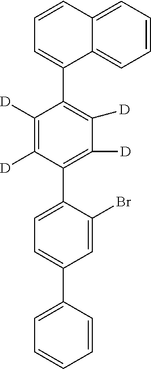 [SMILES: [2H]c1c([2H])c(-c2cccc3ccccc23)c([2H])c([2H])c1-c1ccc(-c2ccccc2)cc1Br] | 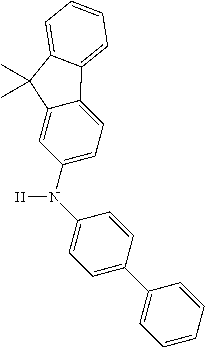 [SMILES: CC1(C)c2ccccc2-c2ccc(Nc3ccc(-c4ccccc4)cc3)cc21] | 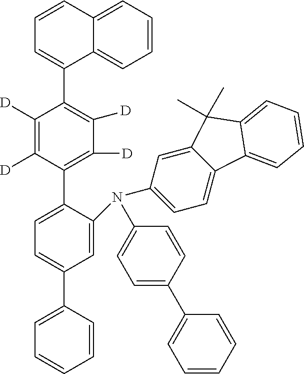 [SMILES: [2H]c1c([2H])c(-c2cccc3ccccc23)c([2H])c([2H])c1-c1ccc(-c2ccccc2)cc1N(c1ccc(-c2ccccc2)cc1)c1ccc2c(c1)C(C)(C)c1ccccc1-2] |
| 2g | 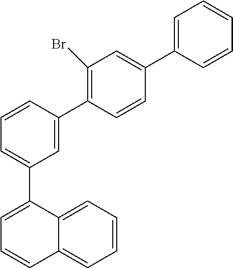 [SMILES: Brc1cc(-c2ccccc2)ccc1-c1cccc(-c2cccc3ccccc23)c1] | 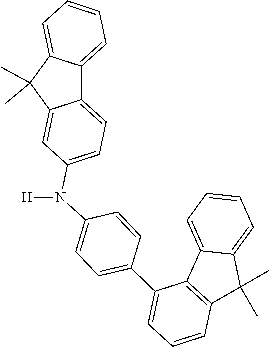 | 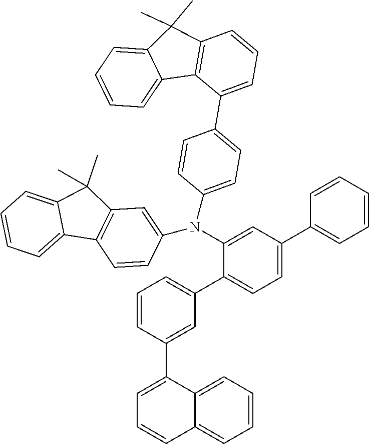 [SMILES: CC1(C)c2ccccc2-c2ccc(N(c3ccc(-c4cccc5c4-c4ccccc4C5(C)C)cc3)c3cc(-c4ccccc4)ccc3-c3cccc(-c4cccc5ccccc45)c3)cc21] |
| 2h | 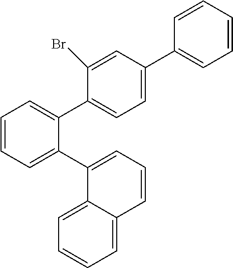 [SMILES: Brc1cc(-c2ccccc2)ccc1-c1ccccc1-c1cccc2ccccc12] | 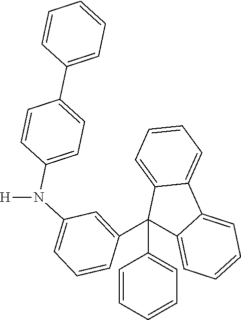 [SMILES: c1ccc(-c2ccc(Nc3cccc(C4(c5ccccc5)c5ccccc5-c5ccccc54)c3)cc2)cc1] | 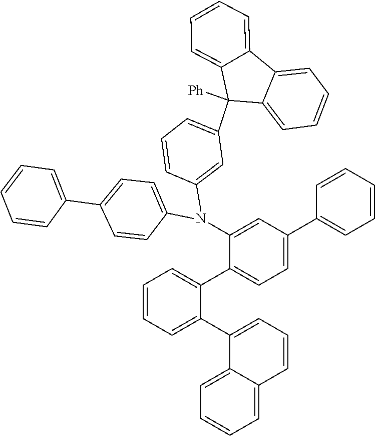 [SMILES: c1ccc(-c2ccc(N(c3cccc(C4(c5ccccc5)c5ccccc5-c5ccccc54)c3)c3cc(-c4ccccc4)ccc3-c3ccccc3-c3cccc4ccccc34)cc2)cc1] |
| 2i | 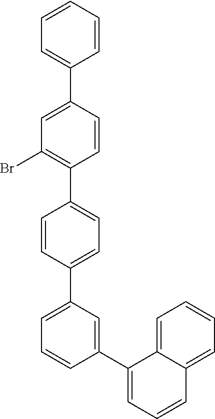 [SMILES: Brc1cc(-c2ccccc2)ccc1-c1ccc(-c2cccc(-c3cccc4ccccc34)c2)cc1] | 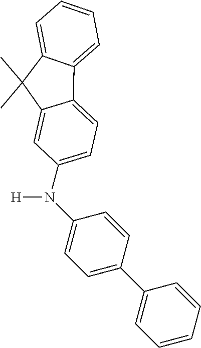 [SMILES: CC1(C)c2ccccc2-c2ccc(Nc3ccc(-c4ccccc4)cc3)cc21] | 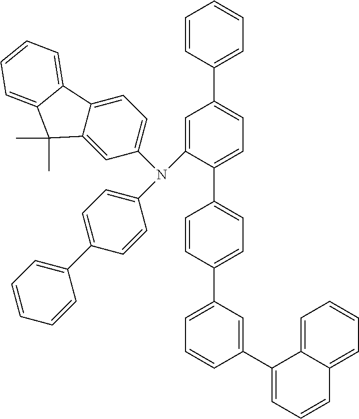 [SMILES: CC1(C)c2ccccc2-c2ccc(N(c3ccc(-c4ccccc4)cc3)c3cc(-c4ccccc4)ccc3-c3ccc(-c4cccc(-c5cccc6ccccc56)c4)cc3)cc21] |
| 2j | 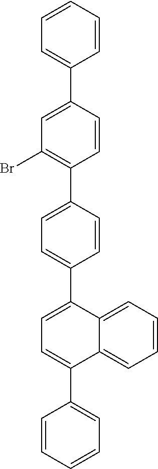 [SMILES: Brc1cc(-c2ccccc2)ccc1-c1ccc(-c2ccc(-c3ccccc3)c3ccccc23)cc1] | 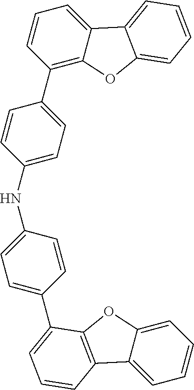 [SMILES: c1ccc2c(c1)oc1c(-c3ccc(Nc4ccc(-c5cccc6c5oc5ccccc56)cc4)cc3)cccc12] | 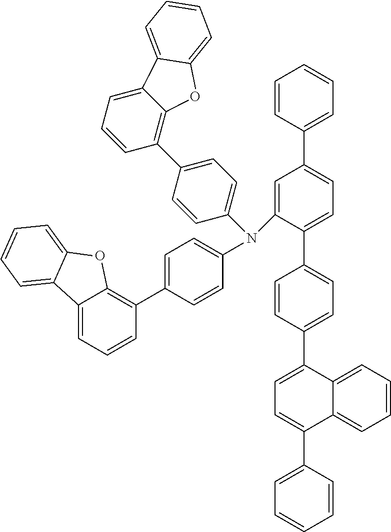 [SMILES: c1ccc(-c2ccc(-c3ccc(-c4ccc(-c5ccccc5)c5ccccc45)cc3)c(N(c3ccc(-c4cccc5c4oc4ccccc45)cc3)c3ccc(-c4cccc5c4oc4ccccc45)cc3)c2)cc1] |
| 2k | 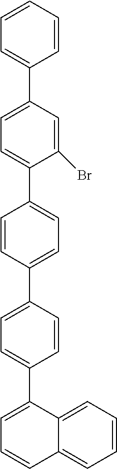 [SMILES: Brc1cc(-c2ccccc2)ccc1-c1ccc(-c2ccc(-c3cccc4ccccc34)cc2)cc1] | 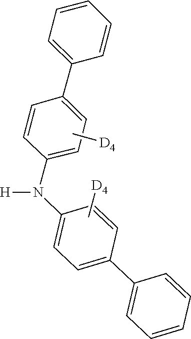 | 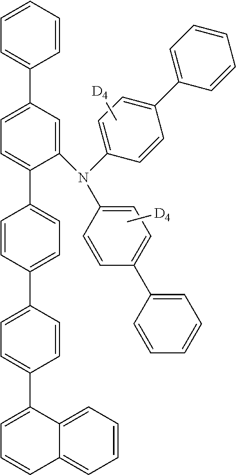 |
| 2l | 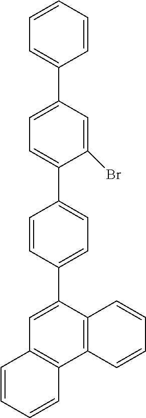 [SMILES: Brc1cc(-c2ccccc2)ccc1-c1ccc(-c2cc3ccccc3c3ccccc23)cc1] | 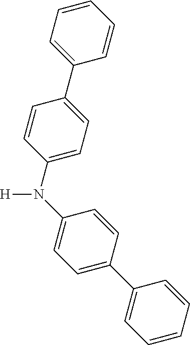 [SMILES: c1ccc(-c2ccc(Nc3ccc(-c4ccccc4)cc3)cc2)cc1] | 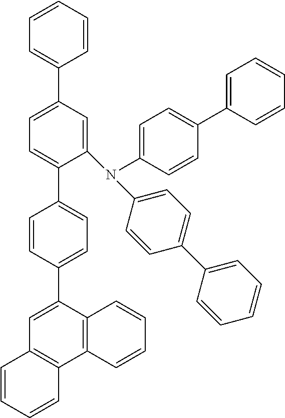 [SMILES: c1ccc(-c2ccc(N(c3ccc(-c4ccccc4)cc3)c3cc(-c4ccccc4)ccc3-c3ccc(-c4cc5ccccc5c5ccccc45)cc3)cc2)cc1] |
| 2m | 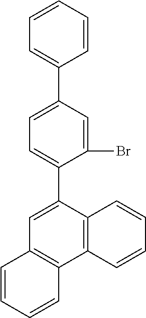 [SMILES: Brc1cc(-c2ccccc2)ccc1-c1cc2ccccc2c2ccccc12] | 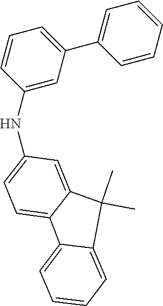 [SMILES: CC1(C)c2ccccc2-c2ccc(Nc3cccc(-c4ccccc4)c3)cc21] | 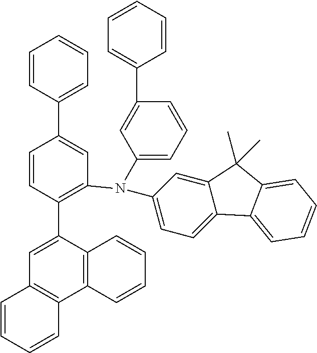 [SMILES: CC1(C)c2ccccc2-c2ccc(N(c3cccc(-c4ccccc4)c3)c3cc(-c4ccccc4)ccc3-c3cc4ccccc4c4ccccc34)cc21] |
| 2o | 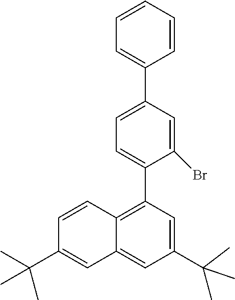 [SMILES: CC(C)(C)c1ccc2c(-c3ccc(-c4ccccc4)cc3Br)cc(C(C)(C)C)cc2c1] | 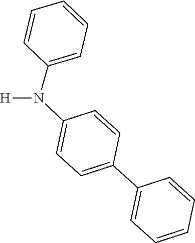 [SMILES: c1ccc(Nc2ccc(-c3ccccc3)cc2)cc1] | 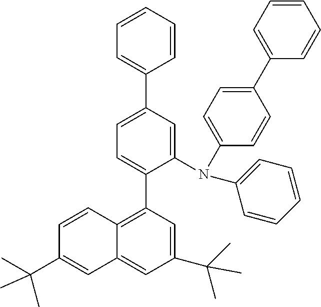 [SMILES: CC(C)(C)c1ccc2c(-c3ccc(-c4ccccc4)cc3N(c3ccccc3)c3ccc(-c4ccccc4)cc3)cc(C(C)(C)C)cc2c1] |
| 2p | 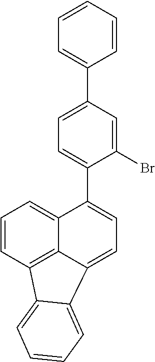 [SMILES: Brc1cc(-c2ccccc2)ccc1-c1ccc2c3c(cccc13)-c1ccccc1-2] | 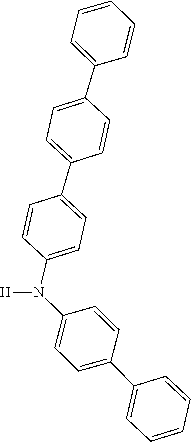 [SMILES: c1ccc(-c2ccc(Nc3ccc(-c4ccc(-c5ccccc5)cc4)cc3)cc2)cc1] | 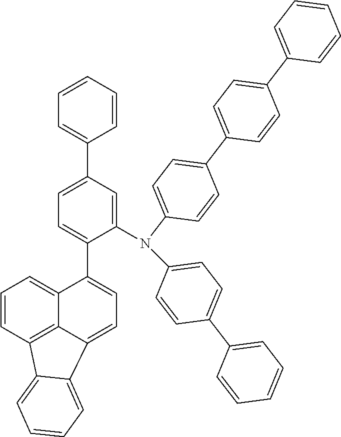 [SMILES: c1ccc(-c2ccc(-c3ccc(N(c4ccc(-c5ccccc5)cc4)c4cc(-c5ccccc5)ccc4-c4ccc5c6c(cccc46)-c4ccccc4-5)cc3)cc2)cc1] |
| 2q | 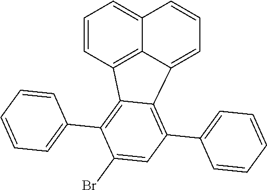 [SMILES: Brc1cc(-c2ccccc2)c2c(c1-c1ccccc1)-c1cccc3cccc-2c13] | 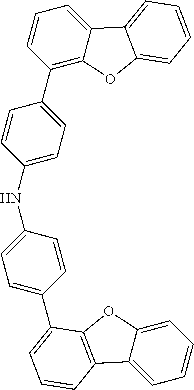 [SMILES: c1ccc2c(c1)oc1c(-c3ccc(Nc4ccc(-c5cccc6c5oc5ccccc56)cc4)cc3)cccc12] | 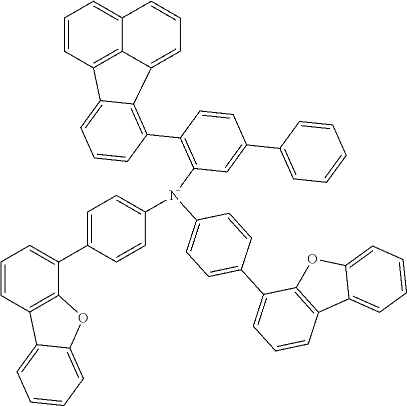 [SMILES: c1ccc(-c2ccc(-c3cccc4c3-c3cccc5cccc-4c35)c(N(c3ccc(-c4cccc5c4oc4ccccc45)cc3)c3ccc(-c4cccc5c4oc4ccccc45)cc3)c2)cc1] |
| 2r | 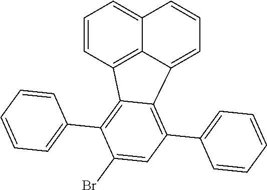 [SMILES: Brc1cc(-c2ccccc2)c2c(c1-c1ccccc1)-c1cccc3cccc-2c13] | 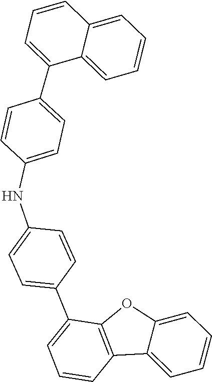 [SMILES: c1ccc2c(-c3ccc(Nc4ccc(-c5cccc6c5oc5ccccc56)cc4)cc3)cccc2c1] | 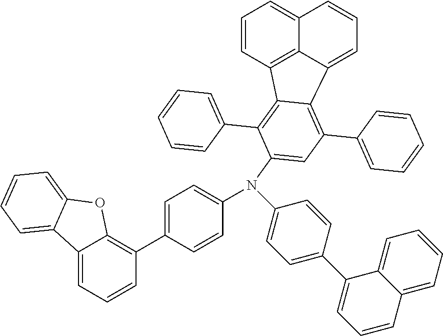 [SMILES: c1ccc(-c2cc(N(c3ccc(-c4cccc5ccccc45)cc3)c3ccc(-c4cccc5c4oc4ccccc45)cc3)c(-c3ccccc3)c3c2-c2cccc4cccc-3c24)cc1] |
| 2s | 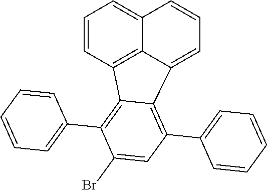 [SMILES: Brc1cc(-c2ccccc2)c2c(c1-c1ccccc1)-c1cccc3cccc-2c13] Synthesis described in WO2021/241900A1 | 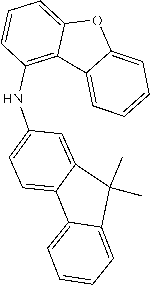 [SMILES: CC1(C)c2ccccc2-c2ccc(Nc3cccc4oc5ccccc5c34)cc21] | 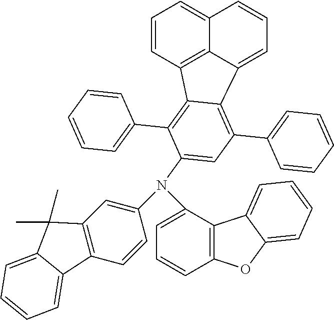 [SMILES: CC1(C)c2ccccc2-c2ccc(N(c3cc(-c4ccccc4)c4c(c3-c3ccccc3)-c3cccc5cccc-4c35)c3cccc4oc5ccccc5c34)cc21] |
| 2t | 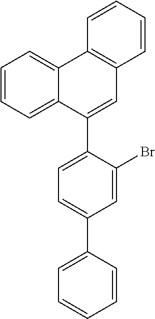 [SMILES: Brc1cc(-c2ccccc2)ccc1-c1cc2ccccc2c2ccccc12] | 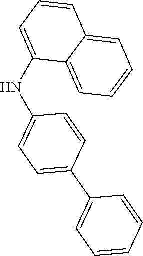 [SMILES: c1ccc(-c2ccc(Nc3cccc4ccccc34)cc2)cc1] | 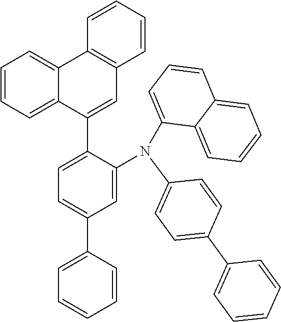 [SMILES: c1ccc(-c2ccc(N(c3cc(-c4ccccc4)ccc3-c3cc4ccccc4c4ccccc34)c3cccc4ccccc34)cc2)cc1] |
| 2u | 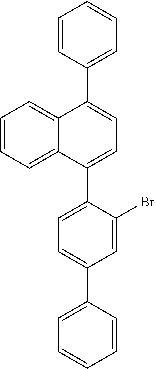 [SMILES: Brc1cc(-c2ccccc2)ccc1-c1ccc(-c2ccccc2)c2ccccc12] | 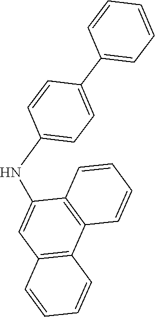 [SMILES: c1ccc(-c2ccc(Nc3cc4ccccc4c4ccccc34)cc2)cc1] | 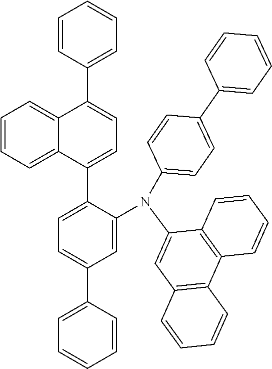 [SMILES: c1ccc(-c2ccc(N(c3cc(-c4ccccc4)ccc3-c3ccc(-c4ccccc4)c4ccccc34)c3cc4ccccc4c4ccccc34)cc2)cc1] |
| 2v | 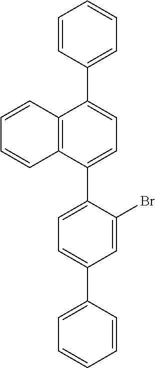 [SMILES: Brc1cc(-c2ccccc2)ccc1-c1ccc(-c2ccccc2)c2ccccc12] | 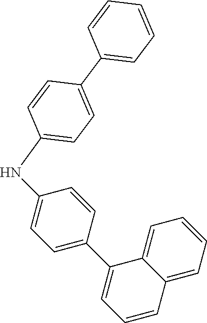 [SMILES: c1ccc(-c2ccc(Nc3ccc(-c4cccc5ccccc45)cc3)cc2)cc1] | 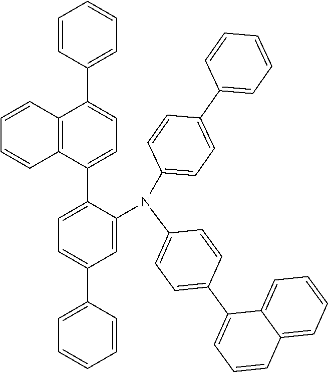 [SMILES: c1ccc(-c2ccc(N(c3ccc(-c4cccc5ccccc45)cc3)c3cc(-c4ccccc4)ccc3-c3ccc(-c4ccccc4)c4ccccc34)cc2)cc1] |
| 2w | 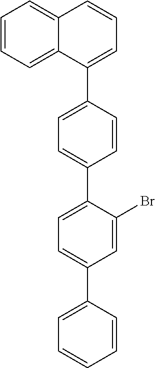 [SMILES: Brc1cc(-c2ccccc2)ccc1-c1ccc(-c2cccc3ccccc23)cc1] | 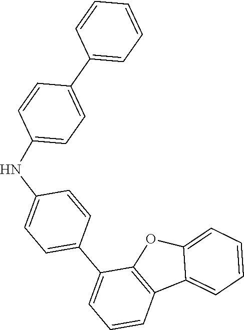 [SMILES: c1ccc(-c2ccc(Nc3ccc(-c4cccc5c4oc4ccccc45)cc3)cc2)cc1] | 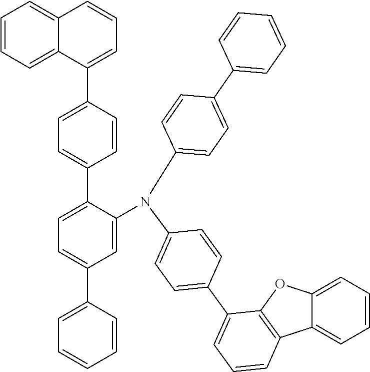 [SMILES: c1ccc(-c2ccc(N(c3ccc(-c4cccc5c4oc4ccccc45)cc3)c3cc(-c4ccccc4)ccc3-c3ccc(-c4cccc5ccccc45)cc3)cc2)cc1] |
| 2x | 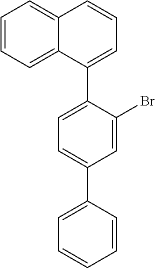 [SMILES: Brc1cc(-c2ccccc2)ccc1-c1cccc2ccccc12] | 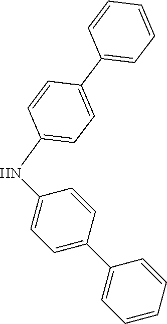 [SMILES: c1ccc(-c2ccc(Nc3ccc(-c4ccccc4)cc3)cc2)cc1] | 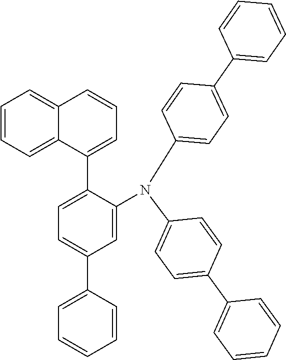 [SMILES: c1ccc(-c2ccc(N(c3ccc(-c4ccccc4)cc3)c3cc(-c4ccccc4)ccc3-c3cccc4ccccc34)cc2)cc1] |

### Synthesis of N-(9,9-dimethyl-9H-fluoren-2-yl)-9,9-dimethyl-N-(4-{4-[4-(naphthalen-1-yl)phenyl]-[1,1′-biphenyl]-3-yl}phenyl)-9H-fluorene-2-amine 3a

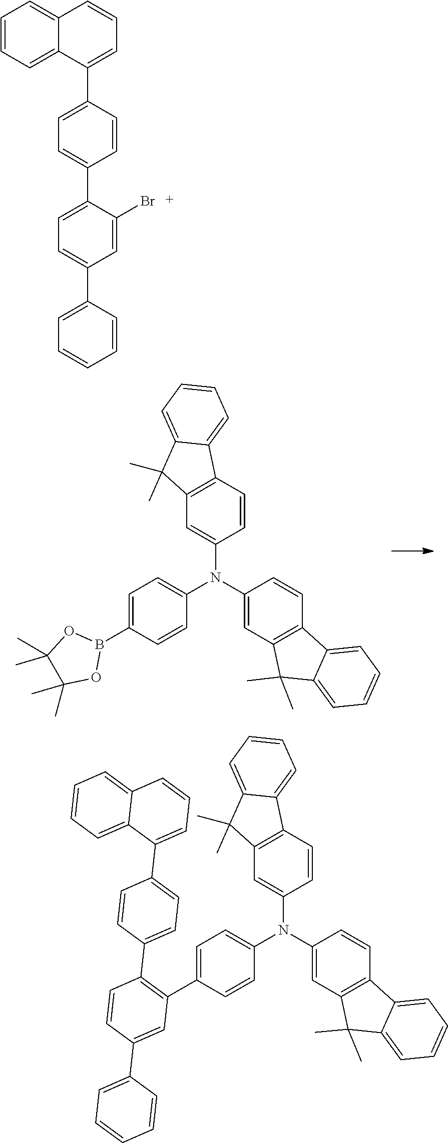

25.9 g (43 mmol) of N-(9,9-dimethyl-9H-fluoren-2-yl)-9,9-dimethyl-N-[4-(4,4,5,5-tetramethyl-1,3,2-dioxaborolan-2-yl)phenyl]-9H-fluorene-2-amine and 18.7 g (43 mmol) of 3-bromo-4-[4-(naphthalen-1-yl)phenyl]-1,1′-biphenyl are suspended in 400 ml of dioxane and 13.7 g of cesium fluoride (90 mmol). 4.0 g (5.4 mmol) of bis(tricyclohexylphosphine) palladium dichloride is added to this suspension, and the reaction mixture is heated under reflux for 18 h. After cooling, the organic phase is removed, filtered through silica gel, washed three times with 80 ml of water and then concentrated to dryness. After the crude product has been filtered through silica gel with toluene, the remaining residue is recrystallized from toluene and finally sublimed under high vacuum; purity is 99.9%. The yield is 12.5 g (35% of theory).

The following compounds are prepared in an analogous manner:

| Ex. | Halogen derivative | Amine | Product |
|---|---|---|---|
| 3b | 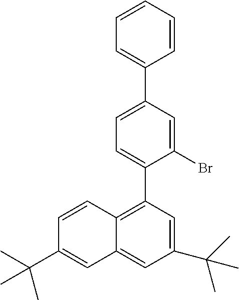 [SMILES: CC(C)(C)c1ccc2c(-c3ccc(-c4ccccc4)cc3Br)cc(C(C)(C)C)cc2c1] | 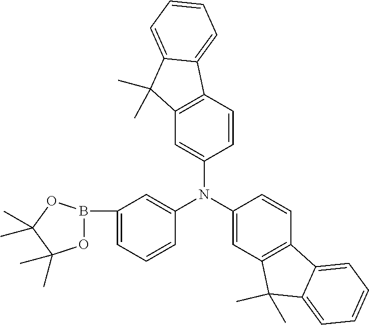 [SMILES: CC1(C)c2ccccc2-c2ccc(N(c3cccc(B4OC(C)(C)C(C)(C)O4)c3)c3ccc4c(c3)C(C)(C)c3ccccc3-4)cc21] | 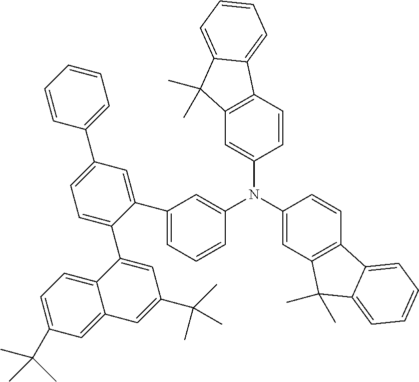 [SMILES: CC(C)(C)c1ccc2c(-c3ccc(-c4ccccc4)cc3-c3cccc(N(c4ccc5c(c4)C(C)(C)c4ccccc4-5)c4ccc5c(c4)C(C)(C)c4ccccc4-5)c3)cc(C(C)(C)C)cc2c1] |
| 3c | 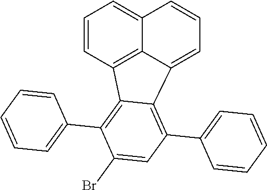 [SMILES: Brc1cc(-c2ccccc2)c2c(c1-c1ccccc1)-c1cccc3cccc-2c13] | 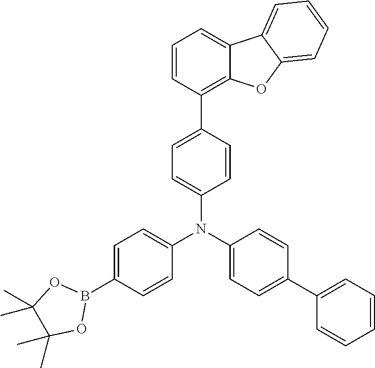 [SMILES: CC1(C)OB(c2ccc(N(c3ccc(-c4ccccc4)cc3)c3ccc(-c4cccc5c4oc4ccccc45)cc3)cc2)OC1(C)C] | 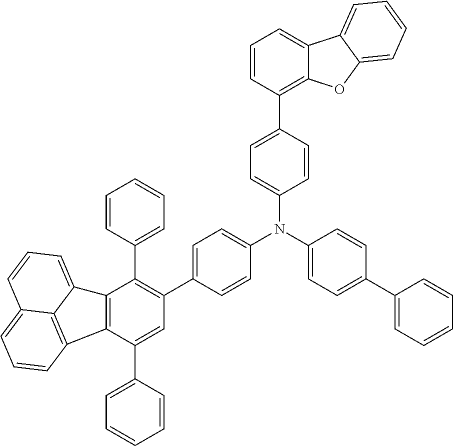 [SMILES: c1ccc(-c2ccc(N(c3ccc(-c4cc(-c5ccccc5)c5c(c4-c4ccccc4)-c4cccc6cccc-5c46)cc3)c3ccc(-c4cccc5c4oc4ccccc45)cc3)cc2)cc1] |
| 3d | 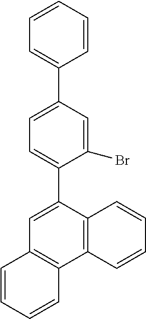 [SMILES: Brc1cc(-c2ccccc2)ccc1-c1cc2ccccc2c2ccccc12] | 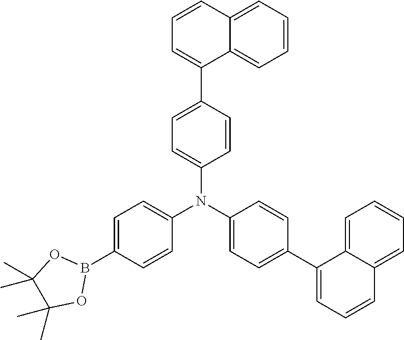 [SMILES: CC1(C)OB(c2ccc(N(c3ccc(-c4cccc5ccccc45)cc3)c3ccc(-c4cccc5ccccc45)cc3)cc2)OC1(C)C] | 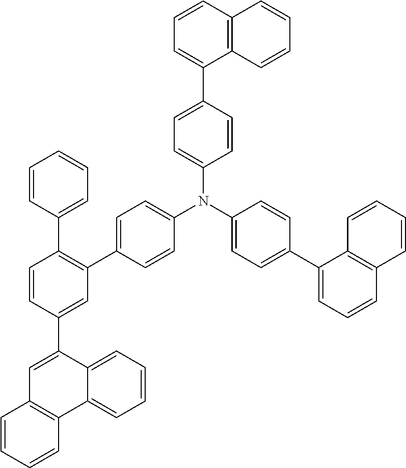 [SMILES: c1ccc(-c2ccc(-c3cc4ccccc4c4ccccc34)cc2-c2ccc(N(c3ccc(-c4cccc5ccccc45)cc3)c3ccc(-c4cccc5ccccc45)cc3)cc2)cc1] |
| 3e | 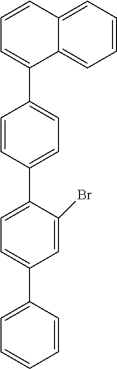 [SMILES: Brc1cc(-c2ccccc2)ccc1-c1ccc(-c2cccc3ccccc23)cc1] | 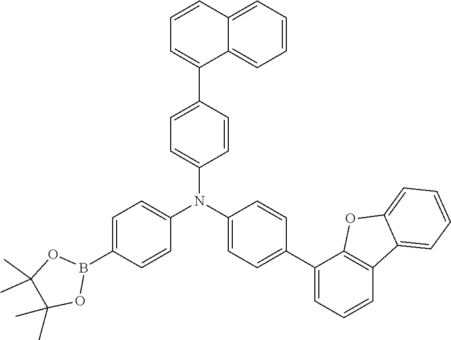 [SMILES: CC1(C)OB(c2ccc(N(c3ccc(-c4cccc5ccccc45)cc3)c3ccc(-c4cccc5c4oc4ccccc45)cc3)cc2)OC1(C)C] | 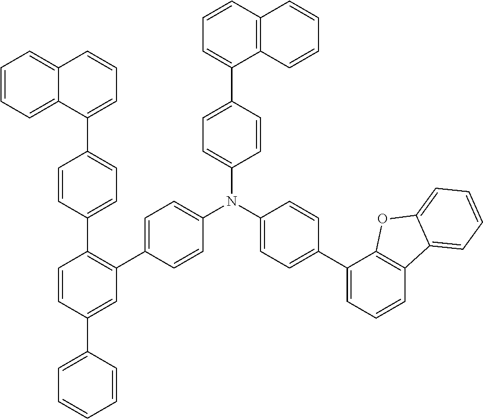 [SMILES: c1ccc(-c2ccc(-c3ccc(-c4cccc5ccccc45)cc3)c(-c3ccc(N(c4ccc(-c5cccc6ccccc56)cc4)c4ccc(-c5cccc6c5oc5ccccc56)cc4)cc3)c2)cc1] |
| 3f | 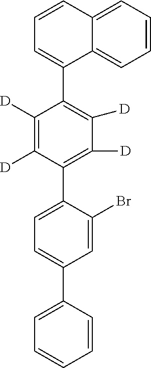 [SMILES: [2H]c1c([2H])c(-c2cccc3ccccc23)c([2H])c([2H])c1-c1ccc(-c2ccccc2)cc1Br] | 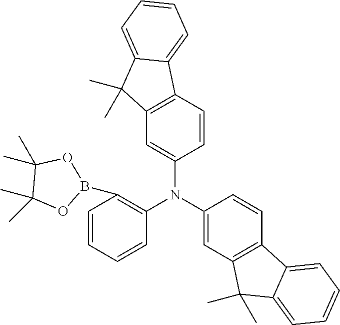 [SMILES: CC1(C)c2ccccc2-c2ccc(N(c3ccc4c(c3)C(C)(C)c3ccccc3-4)c3ccccc3B3OC(C)(C)C(C)(C)O3)cc21] | 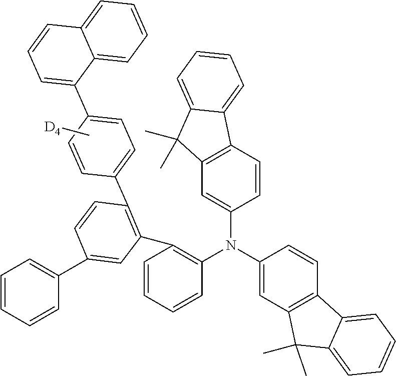 |
| 3g | 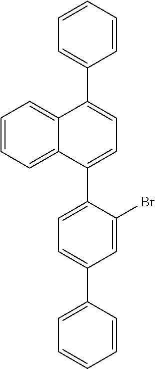 [SMILES: Brc1cc(-c2ccccc2)ccc1-c1ccc(-c2ccccc2)c2ccccc12] | 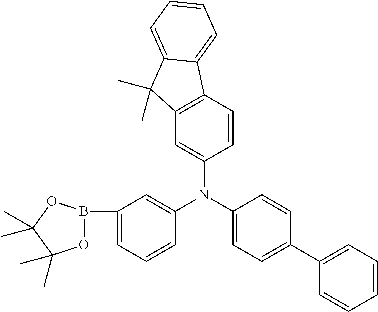 [SMILES: CC1(C)c2ccccc2-c2ccc(N(c3ccc(-c4ccccc4)cc3)c3cccc(B4OC(C)(C)C(C)(C)O4)c3)cc21] | 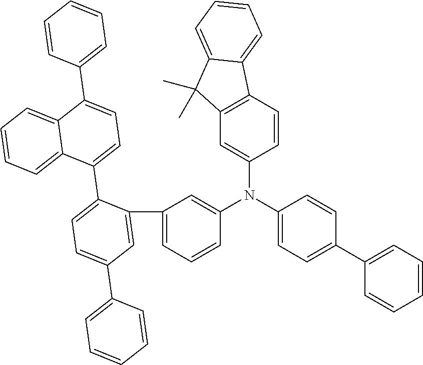 [SMILES: CC1(C)c2ccccc2-c2ccc(N(c3ccc(-c4ccccc4)cc3)c3cccc(-c4cc(-c5ccccc5)ccc4-c4ccc(-c5ccccc5)c5ccccc45)c3)cc21] |
| 3h | 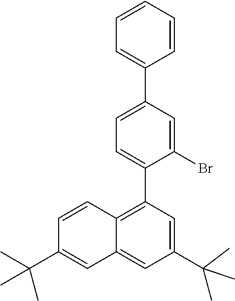 [SMILES: CC(C)(C)c1ccc2c(-c3ccc(-c4ccccc4)cc3Br)cc(C(C)(C)C)cc2c1] | 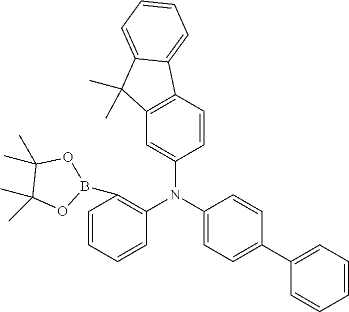 [SMILES: CC1(C)c2ccccc2-c2ccc(N(c3ccc(-c4ccccc4)cc3)c3ccccc3B3OC(C)(C)C(C)(C)O3)cc21] | 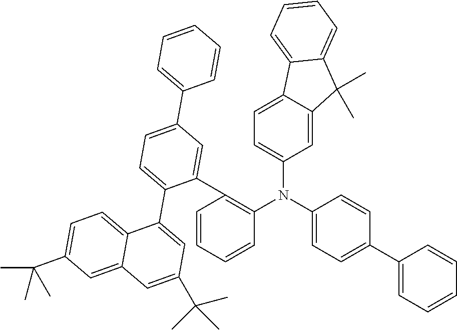 [SMILES: CC(C)(C)c1ccc2c(-c3ccc(-c4ccccc4)cc3-c3ccccc3N(c3ccc(-c4ccccc4)cc3)c3ccc4c(c3)C(C)(C)c3ccccc3-4)cc(C(C)(C)C)cc2c1] |
| 3k | 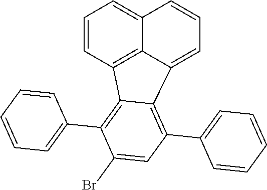 [SMILES: Brc1cc(-c2ccccc2)c2c(c1-c1ccccc1)-c1cccc3cccc-2c13] | 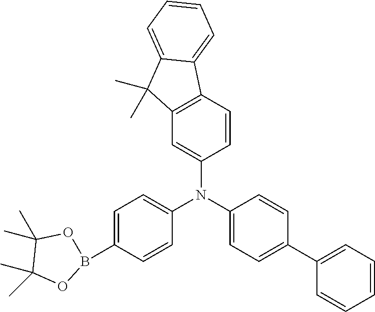 [SMILES: CC1(C)c2ccccc2-c2ccc(N(c3ccc(B4OC(C)(C)C(C)(C)O4)cc3)c3ccc(-c4ccccc4)cc3)cc21] | 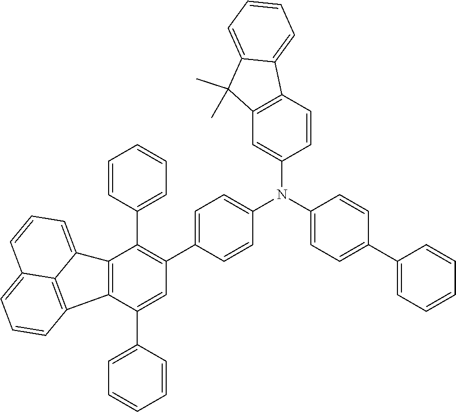 [SMILES: CC1(C)c2ccccc2-c2ccc(N(c3ccc(-c4ccccc4)cc3)c3ccc(-c4cc(-c5ccccc5)c5c(c4-c4ccccc4)-c4cccc6cccc-5c46)cc3)cc21] |
| 3o | 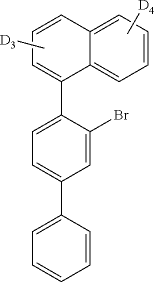 | 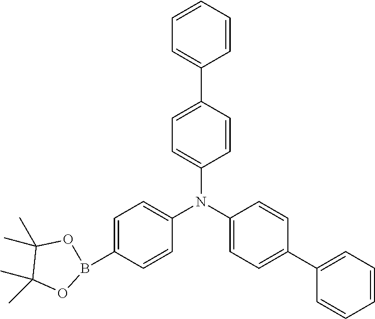 [SMILES: CC1(C)OB(c2ccc(N(c3ccc(-c4ccccc4)cc3)c3ccc(-c4ccccc4)cc3)cc2)OC1(C)C] | 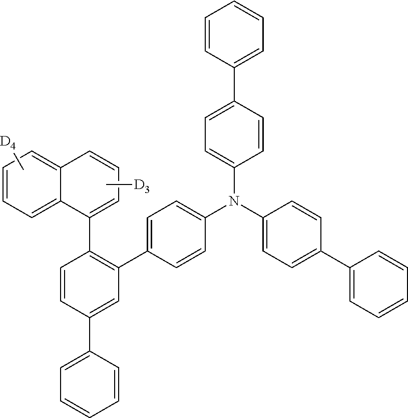 |
| 3p | 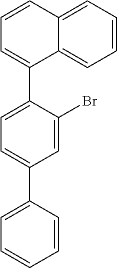 [SMILES: Brc1cc(-c2ccccc2)ccc1-c1cccc2ccccc12] | 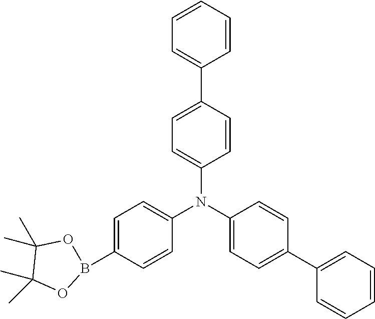 [SMILES: CC1(C)OB(c2ccc(N(c3ccc(-c4ccccc4)cc3)c3ccc(-c4ccccc4)cc3)cc2)OC1(C)C] | 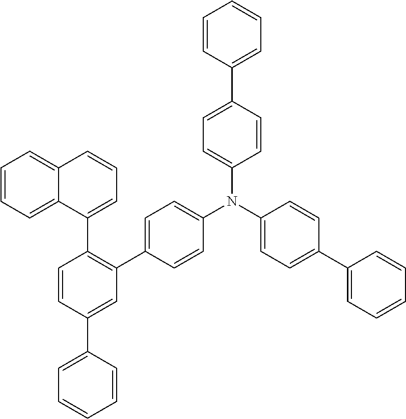 [SMILES: c1ccc(-c2ccc(N(c3ccc(-c4ccccc4)cc3)c3ccc(-c4cc(-c5ccccc5)ccc4-c4cccc5ccccc45)cc3)cc2)cc1] |
| 3q | 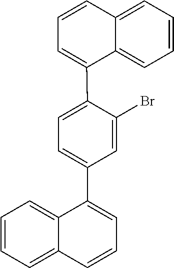 [SMILES: Brc1cc(-c2cccc3ccccc23)ccc1-c1cccc2ccccc12] | 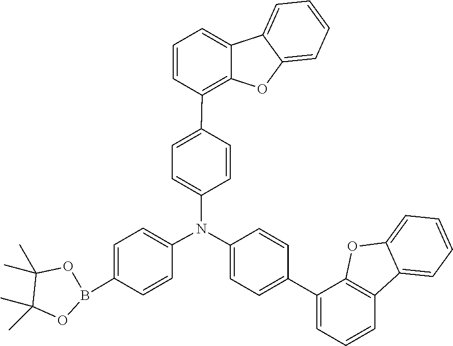 [SMILES: CC1(C)OB(c2ccc(N(c3ccc(-c4cccc5c4oc4ccccc45)cc3)c3ccc(-c4cccc5c4oc4ccccc45)cc3)cc2)OC1(C)C] | 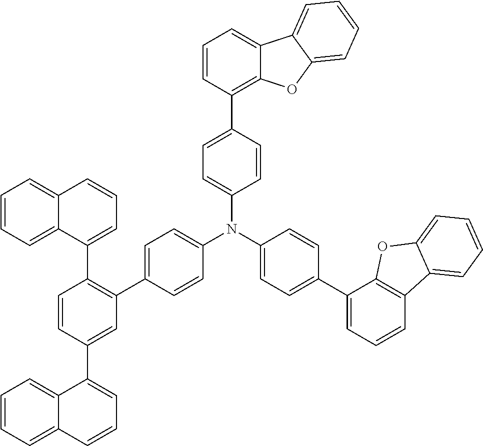 [SMILES: c1ccc2c(-c3ccc(-c4cccc5ccccc45)c(-c4ccc(N(c5ccc(-c6cccc7c6oc6ccccc67)cc5)c5ccc(-c6cccc7c6oc6ccccc67)cc5)cc4)c3)cccc2c1] |
| 3r | 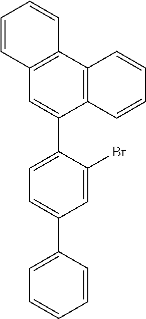 [SMILES: Brc1cc(-c2ccccc2)ccc1-c1cc2ccccc2c2ccccc12] | 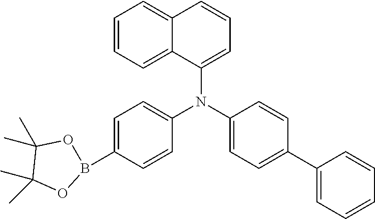 [SMILES: CC1(C)OB(c2ccc(N(c3ccc(-c4ccccc4)cc3)c3cccc4ccccc34)cc2)OC1(C)C] | 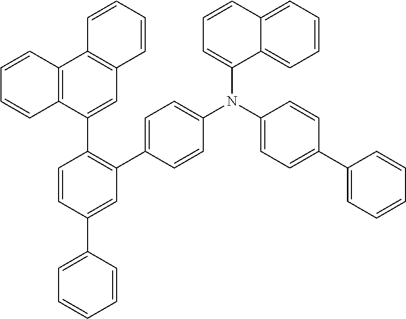 [SMILES: c1ccc(-c2ccc(N(c3ccc(-c4cc(-c5ccccc5)ccc4-c4cc5ccccc5c5ccccc45)cc3)c3cccc4ccccc34)cc2)cc1] |

### B) Device Examples

The compounds according to the application may be used in OLEDs having the following structure:

substrate/hole injection layer (HIL)/hole transport layer (HTL)/electron blocker layer (EBL)/emission layer (EML)/hole blocker layer (HBL)/electron transport layer (ETL)/electron injection layer (EIL)/cathode. The cathode is formed by an aluminum layer of thickness 100 nm. Glass plates which have been coated with structured ITO (indium tin oxide) in a thickness of 50 nm form the substrates to which the OLEDs are applied.

The exact structure of the OLEDs is shown below. The “HTM” material used in the HIL and the HTL is a fluorene derivative. The p-dopant used is NDP-9 from Novaled AG, Dresden.

The compounds according to the application may advantageously be used in the EBL of green-phosphorescing OLEDs, as shown by way of example hereinafter for the compounds HTM-1 and HTM-2:

| | HIL | HTL | EBL | EML | HBL | ETL | EIL |
|---|---|---|---|---|---|---|---|
| | Thick- | Thick- | Thick- | Thick- | Thick- | Thick- | Thick- |
| | ness/ | ness/ | ness/ | ness/ | ness/ | ness/ | ness/ |
| Ex. | nm | nm | nm | nm | nm | nm | nm |
| 1 | HTM:p- | HTM | HTM-1 | TMM-1 | HBM | ETM:LiQ | LiQ |
| | dopant | 50 nm | 30 nm | (32%) | 5 nm | (50%) | 1 nm |
| | (3%) | | | TMM-2 | | 30 nm | |
| | 10 nm | | | (60%) | | | |
| | | | | TEG | | | |
| | | | | (8%) | | | |
| | | | | 35 nm | | | |
| 2 | HTM:p- | HTM | HTM-2 | TMM-1 | HBM | ETM:LiQ | LiQ |
| | dopant | 50 nm | 30 nm | (32%) | 5 nm | (50%) | 1 nm |
| | (3%) | | | TMM-2 | | 30 nm | |
| | 10 nm | | | (60%) | | | |
| | | | | TEG | | | |
| | | | | (8%) | | | |
| | | | | 35 nm | | | |

The following data are obtained for the OLEDs, which show the advantageous use of the compounds:

Measurement results for the OLEDs

| | U @ 10 | EQE @ 10 | LT90 @ 80 |
|---|---|---|---|
| Ex. | mA/cm² (V) | mA/cm² (%) | mA/cm² |
| 1 | 4.1 | 23.2 | 120 |
| 2 | 4.0 | 23.6 | 60 |

The examples that follow show that the compounds according to the application may advantageously find use in the EBL of blue-fluorescing OLEDs.

| | HIL | HTL | EBL | EML | HBL | ETL | EIL |
|---|---|---|---|---|---|---|---|
| | Thick- | Thick- | Thick- | Thick- | Thick- | Thick- | Thick- |
| | ness/ | ness/ | ness/ | ness/ | ness/ | ness/ | ness/ |
| Ex. | nm | nm | nm | nm | nm | nm | nm |
| 3 | HTM:p- | HTM | HTM-1 | H:SEB | HBM | ETM:LiQ | LiQ |
| | dopant | 185 nm | 5 nm | (3%) | 5 nm | (50%) | 1 nm |
| | (3%) | | | 20 nm | | 30 nm | |
| | 10 nm | | | | | | |
| 4 | HTM:p- | HTM | HTM-2 | H:SEB | HBM | ETM:LiQ | LiQ |
| | dopant | 185 nm | 5 nm | (3%) | 5 nm | (50%) | 1 nm |
| | (3%) | | | 20 nm | | 30 nm | |
| | 10 nm | | | | | | |

The following data are obtained for the OLEDs, which show the advantageous use of the compounds:

Measurement results for the OLEDs

| | U @ 10 | EQE @ 10 | LT90 @ 80 |
|---|---|---|---|
| Ex. | mA/cm² (V) | mA/cm² (%) | mA/cm² |
| 3 | 3.6 | 10.4 | 160 |
| 4 | 3.6 | 10.4 | 85 |

Structures of the compounds used 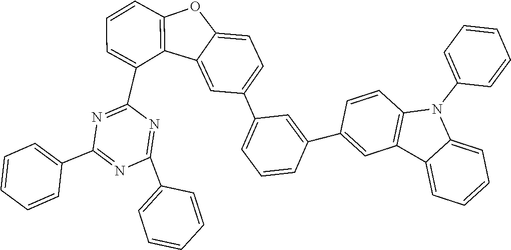 [SMILES: c1ccc(-c2nc(-c3ccccc3)nc(-c3cccc4oc5ccc(-c6cccc(-c7ccc8c(c7)c7ccccc7n8-c7ccccc7)c6)cc5c34)n2)cc1]
TMM-1 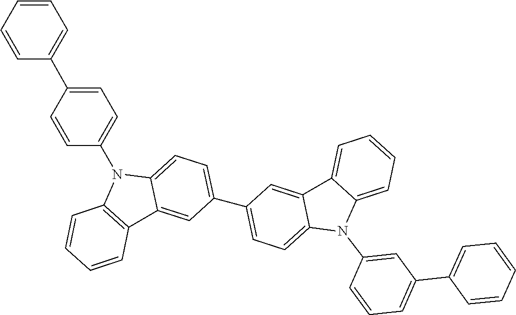 [SMILES: c1ccc(-c2ccc(-n3c4ccccc4c4cc(-c5ccc6c(c5)c5ccccc5n6-c5cccc(-c6ccccc6)c5)ccc43)cc2)cc1]
TMM-2 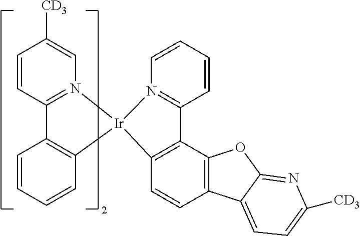
TEG 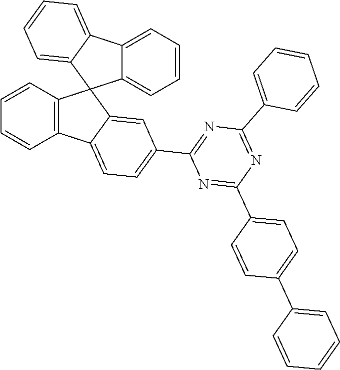 [SMILES: c1ccc(-c2ccc(-c3nc(-c4ccccc4)nc(-c4ccc5c(c4)C4(c6ccccc6-c6ccccc64)c4ccccc4-5)n3)cc2)cc1]
ETM 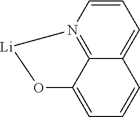
LiQ 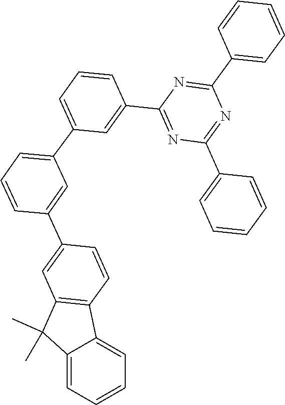 [SMILES: CC1(C)c2ccccc2-c2ccc(-c3cccc(-c4cccc(-c5nc(-c6ccccc6)nc(-c6ccccc6)n5)c4)c3)cc21]
HBM 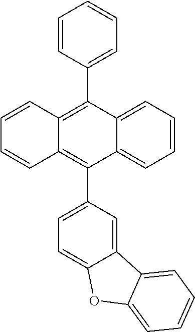 [SMILES: c1ccc(-c2c3ccccc3c(-c3ccc4oc5ccccc5c4c3)c3ccccc23)cc1]
H 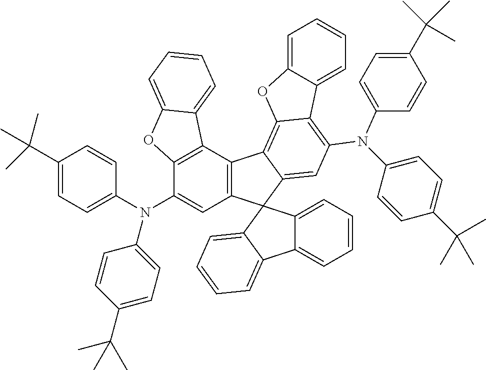 [SMILES: CC(C)(C)c1ccc(N(c2ccc(C(C)(C)C)cc2)c2cc3c(c4c2oc2ccccc24)-c2c(cc(N(c4ccc(C(C)(C)C)cc4)c4ccc(C(C)(C)C)cc4)c4c2oc2ccccc24)C32c3ccccc3-c3ccccc32)cc1]
SEB 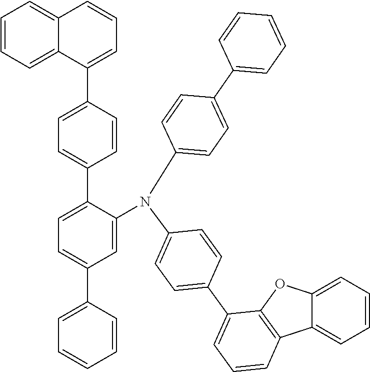 [SMILES: c1ccc(-c2ccc(N(c3ccc(-c4cccc5c4oc4ccccc45)cc3)c3cc(-c4ccccc4)ccc3-c3ccc(-c4cccc5ccccc45)cc3)cc2)cc1]
HTM-1 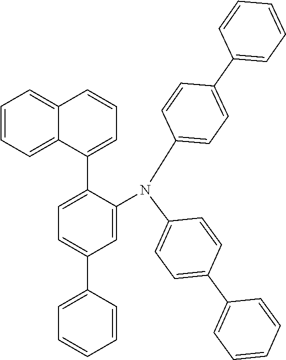 [SMILES: c1ccc(-c2ccc(N(c3ccc(-c4ccccc4)cc3)c3cc(-c4ccccc4)ccc3-c3cccc4ccccc34)cc2)cc1]
HTM-2

The following compounds HTM-3 to HTM-8 may likewise advantageously be used in the above setups, in place of HTM-1 and HTM-2, and give comparable device measurement results:

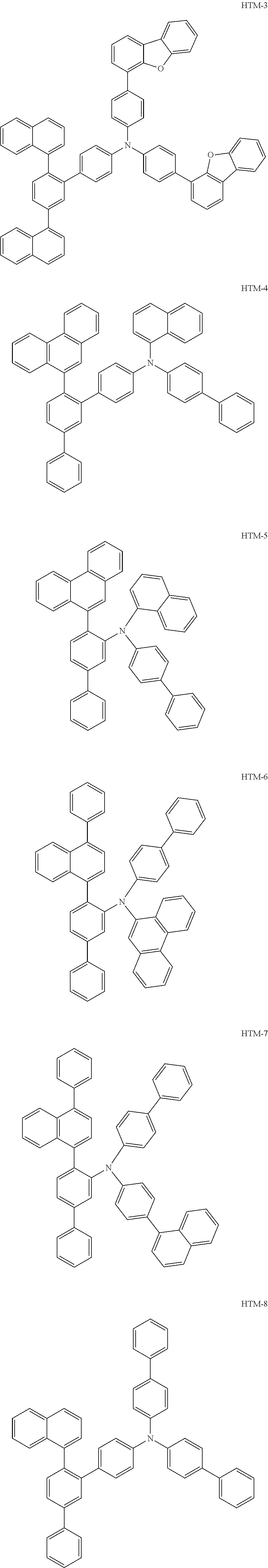

Compounds HTM-3 to HTM-8 are usable in particular for use in the setup of examples 1 and 2 shown above, in place of compound HTM-1 or HTM-2, and give comparable results to those shown above for compounds HTM-1 and HTM-2.

1.-18. (canceled)

19. A compound of a formula (I) 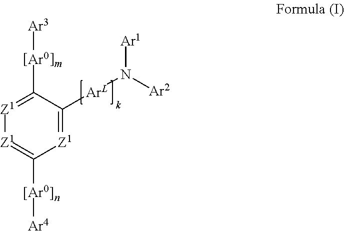
where the variables that occur are as follows:
Z¹ is the same or different at each instance and is selected from N and CR¹; where the unit 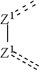
may be replaced by a unit selected from units of the following formulae: 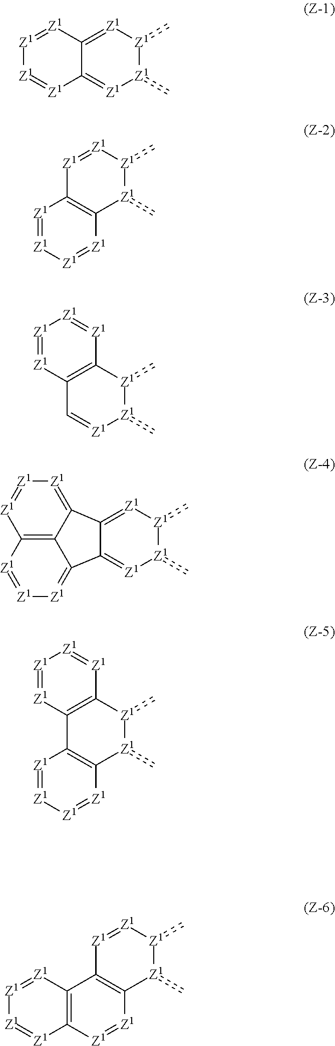 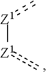
where the dotted bonds in the units each correspond to the bonds in the unit 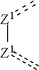
and where
the variables Z¹ in the unit in these cases are C;
Ar⁰ is phenylene substituted by R² radicals;
Ar¹ and Ar² are the same or different at each instance and are selected from aromatic ring systems which have 6 to 40 aromatic ring atoms and are substituted by R⁴ radicals, and from heteroaromatic ring systems which have 5 to 40 aromatic ring atoms and are substituted by R⁴ radicals;
Ar³ and Ar⁴ are the same or different at each instance and are selected from aromatic ring systems which have 6 to 40 aromatic ring atoms and are substituted by R⁵ radicals, and from heteroaromatic ring systems which have 5 to 40 aromatic ring atoms and are substituted by R⁵ radicals;
where at least one of Ar³ and Ar⁴ is selected from aryl groups which have 10 to 20 aromatic ring atoms and are substituted by R⁵ radicals;
Ar^{L} is selected from aromatic ring systems which have 6 to 40 aromatic ring atoms and are substituted by R³ radicals and from heteroaromatic ring systems which have 5 to 40 aromatic ring atoms and are substituted by R³ radicals;
R¹ is the same or different at each instance and is selected from H, D, F, Cl, Br, I, C(═O)R⁶, CN, Si(R⁶)₃, N(R⁶)₂, P(═O)(R⁶)₂, OR⁶, S(═O)R⁶, S(═O)₂R⁶, straight-chain alkyl or alkoxy groups having 1 to 20 carbon atoms, branched or cyclic alkyl or alkoxy groups having 3 to 20 carbon atoms, alkenyl or alkynyl groups having 2 to 20 carbon atoms, where said alkyl, alkoxy, alkenyl and alkynyl groups are each substituted by R⁶ radicals; and where one or more CH₂ groups in said alkyl, alkoxy, alkenyl and alkynyl groups may be replaced by —R⁶C≡CR⁶—, —C≡C—, Si(R⁶)₂, C═O, C═NR⁶, —C(═O)O—, —C(═O)NR⁶—, NR⁶, P(═O)(R⁶), —O—, —S—, SO or SO₂;
R⁴ is the same or different at each instance and is selected from H, D, F, Cl, Br, I, C(═O)R⁶, CN, Si(R⁶)₃, P(═O)(R⁶)₂, OR⁶, S(═O)R⁶, S(═O)₂R⁶, straight-chain alkyl or alkoxy groups having 1 to 20 carbon atoms, branched or cyclic alkyl or alkoxy groups having 3 to 20 carbon atoms, alkenyl or alkynyl groups having 2 to 20 carbon atoms, aromatic ring systems having 6 to 40 aromatic ring atoms, and heteroaromatic ring systems having 5 to 40 aromatic ring atoms; where two or more R⁴ radicals may be joined to one another and may form a ring; where said alkyl, alkoxy, alkenyl and alkynyl groups and said aromatic ring systems and heteroaromatic ring systems are each substituted by R⁶ radicals; and where one or more CH₂ groups in said alkyl, alkoxy, alkenyl and alkynyl groups may be replaced by —RC≡CR⁶—, —C≡C—, Si(R⁶)₂, C═O, C═NR⁶, —C(═O)O—, —C(═O)NR⁶—, NR⁶, P(═O)(R⁶), —O—, —S—, SO or SO₂;
R², R³ and R⁵ are the same or different at each instance and are selected from H, D, F, Cl, Br, I, C(═O)R⁶, CN, Si(R⁶)₃, N(R⁶)₂, P(═O)(R⁶)₂, OR⁶, S(═O)R⁶, S(═O)₂R⁶, straight-chain alkyl or alkoxy groups having 1 to 20 carbon atoms, branched or cyclic alkyl or alkoxy groups having 3 to 20 carbon atoms, alkenyl or alkynyl groups having 2 to 20 carbon atoms, aromatic ring systems having 6 to 40 aromatic ring atoms, and heteroaromatic ring systems having 5 to 40 aromatic ring atoms; where two or more radicals selected from the R², R³ and R⁵ radicals may be joined to one another and may form a ring; where said alkyl, alkoxy, alkenyl and alkynyl groups and said aromatic ring systems and heteroaromatic ring systems are each substituted by R⁶ radicals; and where one or more CH₂ groups in said alkyl, alkoxy, alkenyl and alkynyl groups may be replaced by —R⁶C—CR⁶—, —C≡C—, Si(R⁶)₂, C—O, C═NR⁶, —C(═O)O—, —C(═O)NR⁶—, NR⁶, P(═O)(R⁶), —O—, —S—, SO or SO₂;
R⁶ is the same or different at each instance and is selected from H, D, F, Cl, Br, I, C(═O)R⁷, CN, Si(R⁷)₇, P(═O)(R⁷)₂, OR⁷, S(═O)R⁷, S(═O)₂R⁷, straight-chain alkyl or alkoxy groups having 1 to 20 carbon atoms, branched or cyclic alkyl or alkoxy groups having 3 to 20 carbon atoms, alkenyl or alkynyl groups having 2 to 20 carbon atoms, aromatic ring systems having 6 to 40 aromatic ring atoms, and heteroaromatic ring systems having 5 to 40 aromatic ring atoms; where two or more R⁶ radicals may be joined to one another and may form a ring; where the alkyl, alkoxy, alkenyl and alkynyl groups mentioned and the aromatic ring systems and heteroaromatic ring systems mentioned are each substituted by R³ radicals; and where one or more CH₂ groups in the alkyl, alkoxy, alkenyl and alkynyl groups mentioned may be replaced by —R⁷C≡CR⁷—, —C≡C—, Si(R⁷)₂, C═O, C═NR⁷, —C(═O)O—, —C(═O)NR⁷—, NR⁷, P(═O)(R⁷), —O—, —S—, SO or SO₂;
R⁷ is the same or different at each instance and is selected from H, D, F, Cl, Br, I, CN, alkyl or alkoxy groups having 1 to 20 carbon atoms, alkenyl or alkynyl groups having 2 to 20 carbon atoms, aromatic ring systems having 6 to 40 aromatic ring atoms and heteroaromatic ring systems having 5 to 40 aromatic ring atoms; where two or more R⁷ radicals may be joined to one another and may form a ring; and where said alkyl, alkoxy, alkenyl and alkynyl groups, aromatic ring systems and heteroaromatic ring systems may be substituted by one or more radicals selected from F and CN;
m is 0, 1, 2 or 3;
n is 0, 1, 2 or 3;
k is 0 or 1.

20. A compound as claimed in claim 19, characterized in that all Z¹ groups are CR¹, and in that 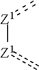
units cannot be replaced by units of the formulae (Z-1) to (Z-6).

21. A compound as claimed in claim 19, characterized in that index m is 0 or 1.

22. A compound as claimed in claim 19, characterized in that index n is 0 or 1.

23. A compound as claimed in claim 19, characterized in that at least one index selected from indices m and n is greater than 0.

24. A compound as claimed in claim 19, characterized in that index k=0, or in that index k=1 and Ar^{L} is phenyl substituted by R³ radicals.

25. A compound as claimed in claim 19, characterized in that Ar³ and Ar⁴ are the same or different at each instance and are selected from phenyl, biphenyl, naphthyl, phenanthrenyl, fluoranthenyl, triphenylenyl and fluorenyl, where the groups are each substituted by R⁵ radicals.

26. A compound as claimed in claim 19, characterized in that the at least one group selected from Ar³ and Ar⁴ groups that must be selected from aryl groups which have 10 to 20 aromatic ring atoms and are substituted by R⁵ radicals is selected from groups of the following formulae: 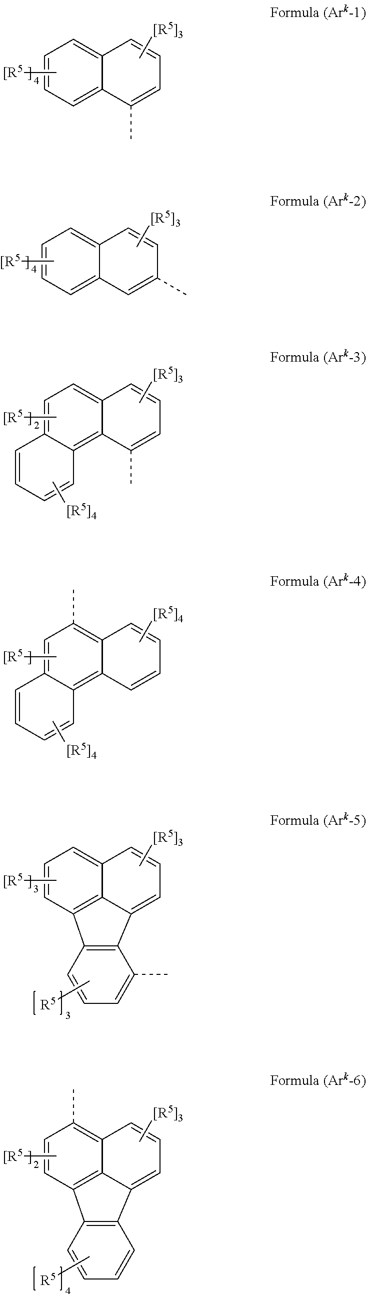
where the variables that occur are as defined in claim 19, and where the dotted line is the bond of the Ar³ or Ar⁴ group to the rest of the compound.

27. A compound as claimed in claim 19, characterized in that
R¹ is the same or different at each instance and is selected from H, D, F, CN, Si(R⁶)₃, N(R⁶)₂, straight-chain alkyl groups having 1 to 20 carbon atoms, and branched or cyclic alkyl groups having 3 to 20 carbon atoms; where the alkyl groups mentioned are each substituted by R⁶ radicals and where one or more CH₂ groups in said alkyl groups may be replaced by —C≡C—, —R⁶C≡CR⁶—, Si(R⁶)₂, C═O, C═NR⁶, —NR⁶—, —O—, —S—, —C(═O)O— or —C(═O)NR⁶—; and
—R², R³ and R⁵ are the same or different at each instance and are selected from H, D, F, CN, straight-chain alkyl groups having 1 to 20 carbon atoms, branched or cyclic alkyl groups having 3 to 20 carbon atoms, and aromatic ring systems having 6 to 40 aromatic ring atoms; where said alkyl groups and said aromatic ring systems are each substituted by R⁶ radicals.

28. A compound as claimed in claim 19, characterized in that the compound conforms to one of the following formulae: 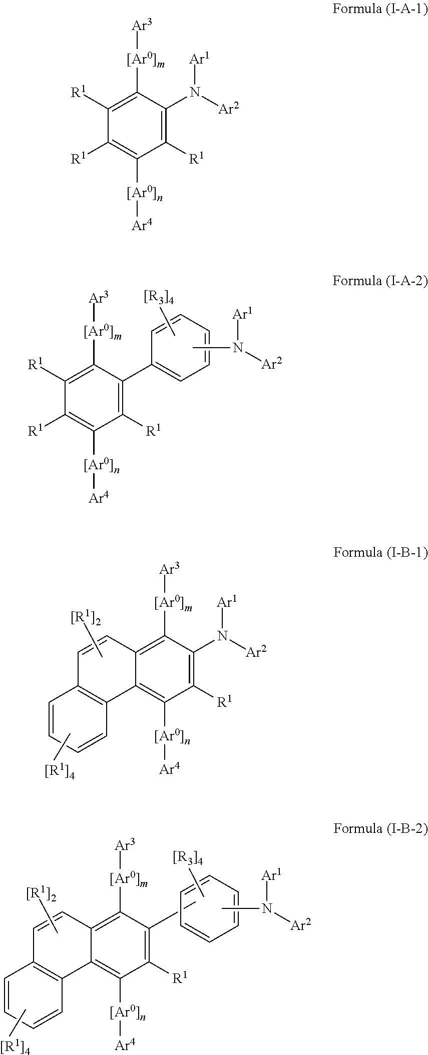 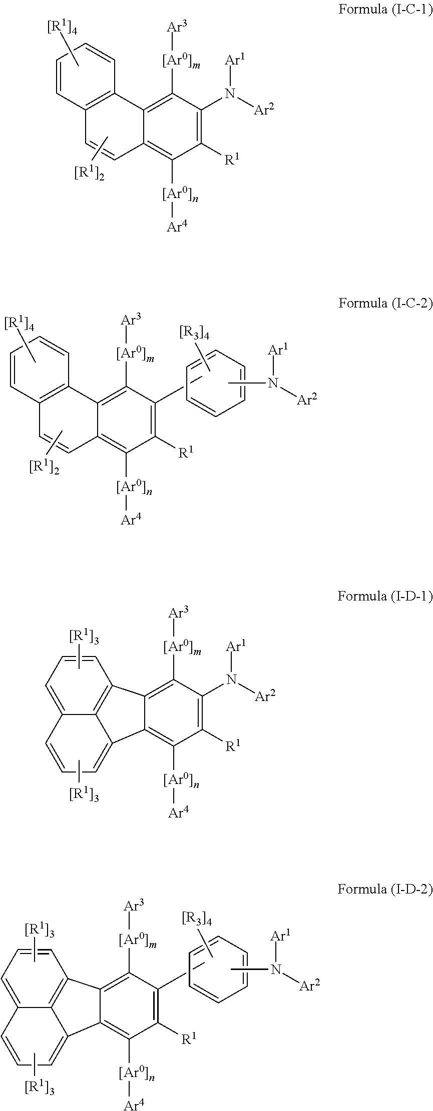
where the variables that occur are as defined in claim 19.

29. A compound as claimed in claim 19, characterized in that Ar⁴ is phenyl substituted by R⁵ radicals.

30. A compound as claimed in claim 19, characterized in that it conforms to one of the following formulae: 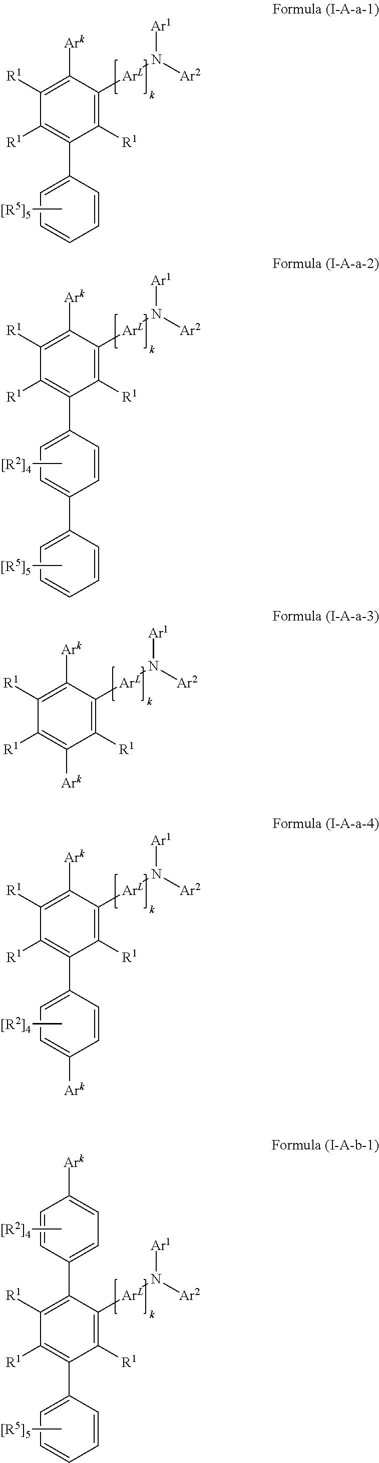 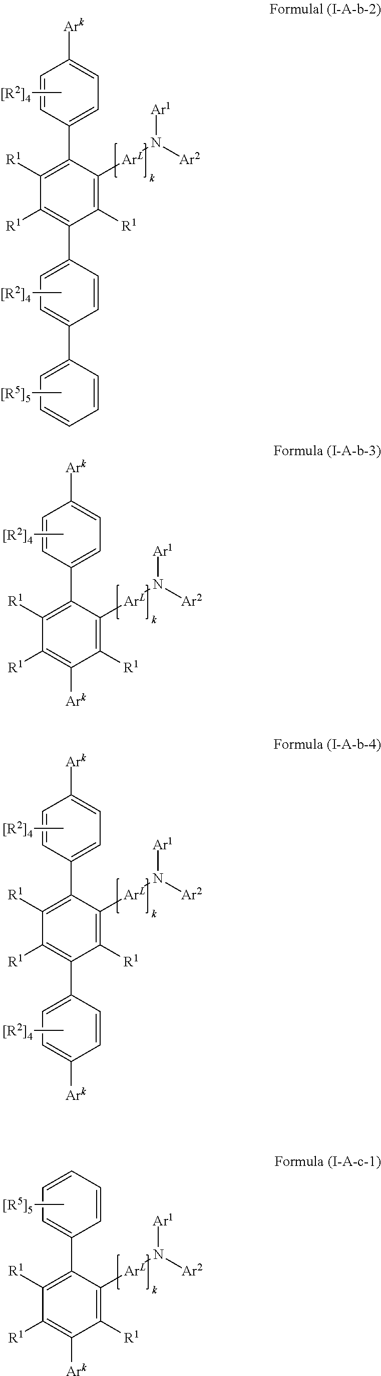 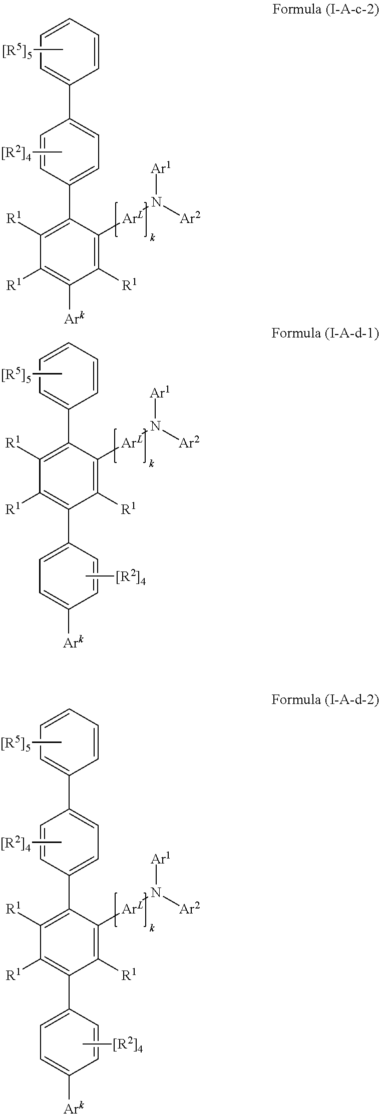
where Ar^{k} is selected from aryl groups which have 10 to 20 aromatic ring atoms and are substituted by R⁵ radicals.

31. A process for preparing a compound as claimed in claim 19, characterized in that a catenated trisaryl derivative substituted by a reactive group is either a) reacted in a coupling reaction with a secondary amine, or b) reacted in a coupling reaction with an aromatic or heteroaromatic species having a boron-containing group and having an amino group.

32. An oligomer, polymer or dendrimer containing one or more compounds as claimed in claim 19, wherein the bond(s) to the polymer, oligomer or dendrimer may be localized at any desired positions substituted by R¹, R², R³, R⁴ or R⁵ in the formulae.

33. A formulation comprising at least one compound as claimed in claim 19 and at least one solvent.

34. An electronic device comprising at least one compound as claimed in claim 19.

35. The electronic device as claimed in claim 34, characterized in that it is an organic electroluminescent device and comprises an anode, cathode and at least one emitting layer, and in that the compound is present in a hole-transporting layer or in an emitting layer of the device.

36. The use of a compound as claimed in claim 19 in an electronic device.
